(19) United States
(12) Patent Application Publication
(10) Pub. No.: US 2006/0099583 A1
(43) Pub. Date: May 11, 2006

(54) Compositions and methods for identifying antiviral agents

(76) Inventor: Scott E Devine, Atlanta, GA (US)

Correspondence Address: FISH & RICHARDSON PC, P.O. BOX 1022, MINNEAPOLIS, MN, 55440-1022, US

(21) Appl. No.: 10/512,789

(22) PCT Filed: May 7, 2003
(86) PCT No.: PCT/US03/14382

§ 371 Date: Oct. 6, 2005

Related U.S. Application Data
(60) Provisional application No. 60/378,711, filed on May 7, 2002.

(51) Int. Cl. C12Q 1/68; G01N 33/53

(52) U.S. Cl. 435/6; 435/7.1

(57) ABSTRACT

Disclosed are compositions and methods that can be used to identify antiviral compounds. The methods can be carried out by exposing a cell that expresses a host factor to a candidate compound. If the expression or activity of the host factor, which is a protein we identified by virtue of its influence on the endogenous retrovirus-like Ty1 element in yeast, is inhibited, the candidate compound is a potential antiviral agent. Such agents can be further tested, if desired, by determining whether they inhibit the ability of the virus to infect a cell or replicate within it.

### CROSS REFERENCE TO RELATED APPLICATIONS

This application claims the benefit of the priority date of U.S. Provisional Application No. 60/378,711, which was filed on May 7, 2002. For the purpose of any national phase application that is subsequently prosecuted in the United States, the entire content of the provisional application is incorporated herein by reference.

### TECHNICAL FIELD

This invention relates to compositions and methods for identifying antiviral agents, including those that are effective against retroviruses, such as human immunodeficiency viruses.

### BACKGROUND

Retroviruses cause diseases such as acquired immune deficiency syndrome (AIDS), and they also play a causative role in cancer. Retroviruses generally encode Gag and Pol as well as additional proteins that are required to carry out their life cycles. These life cycles are complex, and they include (1) the assembly of virus particles (2) reverse transcription of mRNA and (3) integration of cDNA into the genome. Given the increasing prevalence of retroviral diseases, there is a need for new anti-viral strategies and treatments for retroviral diseases. There is also a need for new methods to identify such antiviral compounds and treatments.

### SUMMARY OF THE INVENTION

The present invention is based, in part, on studies that exploited a collection of gene deletion mutants to identify proteins in yeast cells that influence the endogenous retrovirus-like Ty1 element (these proteins are referred to below as “host factors”). As described further below, Ty1 is a retrotransposon (sometimes called a retroposon) present in yeast, that is related to retroviruses; Ty1 uses a mechanism similar to that used by retroviruses to integrate into the genome of a host cell. In our studies, we identified 105 yeast genes and the sequences of human proteins that are homologous to the host factors encoded by many of these yeast genes. At least 27 of the yeast host factors had significant homology to human proteins (with BLAST Expect values of <10⁻³⁰). The Ty1 host factors identified in yeast can be used to study Ty1 and identify antiviral agents. Homologous proteins in higher organisms, such as the human homologs shown in FIG. 4, can also be used to identify antiviral agents. Accordingly, the present invention features methods of screening agents for antiretroviral activity and compositions useful in such screens (e.g. collections of host factors and cells in which one or more host factors have been inactivated). As described further below, the screening methods can be designed to detect a change (e.g., a decrease) in the expression or activity of a host factor. Expression can be detected by any of the methods presently known in the art (e.g., Northern blot assays, RT-PCR or other PCR-based amplification assays, RNAse protection assays, or in antibody-based assays (where the expression measured is protein expression, rather than gene expression), etc.; expression can also be examined in microarrays). Activity can similarly be measured by known assays and techniques (e.g., kinase assays, cellular proliferation assays, etc.).

As used herein, a “host factor” is a yeast protein encoded by a gene identified in Table 1, a human homolog thereof (including those shown in FIG. 4), a homologous protein in another animal, or a fragment, other mutant (e.g. a substitution mutant), or derivative (e.g., a protein encoded by a splice variant or a protein to which additional amino acids residues have been attached) of any of these proteins. Where the host factor is not naturally occurring, it must retain one or more of the biological activities of the corresponding wild type host factor or it must function in the methods described herein. Homologous proteins (e.g. a mouse homolog or a homolog from a non-human primate) and fragments, other mutants, and derivatives of host proteins can be identified by their ability to function in a manner that is substantially equivalent to the yeast and human host factors described herein. A given protein will function in a manner that is substantially equivalent to that of a yeast or human host factor described herein if it exhibits one or more of the known, natural functions of the host factors (see FIG. 5) or if it works in one or more of the screening assays set forth below. For example, a protein that constitutes a fragment of the protein encoded by ARD1 or a fragment of SEQ ID NO:16 (a human homolog of the protein encoded by ARD1) is a host factor so long as it can be used in place of (i.e., can effectively substitute for) the protein naturally encoded by ARD1 or the protein represented by SEQ ID NO:16 in one of the assays described herein for identifying antiviral agents. This is not to say that the homologous, mutant, or variant protein need exhibit activity as robust as that of its wildtype counterpart. Retention of even a small amount of the activity is sufficient so long as the homolog, mutant or variant protein is useful in detecting antiviral agents.

As illustrated further in the Examples below, Ard1/Nat1 encode a heterodimeric acetyltransferase. Together, these proteins modify target proteins, adding a chemical moiety to their N-termini. When working with the host factor Ard1, one could screen for compounds that bind to Ard1 or that inhibit the N-terminal acetylase activity using, for example, a substrate such as a histone. For example, one could monitor the incorporation of a radiolabeled acetyl group. Alternatively, one could assay for dimerization between Ard1 and Nat1 or for other known in vivo functions of Ard1 and/or Nat1. Such functions include teleomeric silencing and cell cycle progression (see FIG. 5). Analogous assays can be used to test any of the factors for which a biological function or property (e.g. dimerization) is known or can be ascertained.

An “antiviral agent” is an agent that inhibits a virus in any therapeutically beneficial way (the antiviral agents identified using the compositions and methods described herein are expected to inhibit retroviruses (e.g., those that infect humans and domesticated animals, such as cats) although the agents identified may have other therapeutic uses as well (e.g., they may be useful in inhibiting viruses other than retroviruses)). For example, an antiviral agent can inhibit the ability of a retrovirus to infect cells, replicate within them, propagate, or infect secondary cells and can, as a consequence, improve a clinical sign or symptom in a patient who is infected with the retrovirus. The agent may also provide benefits to patients who have not yet been infected by reducing the likelihood that they will become infected following exposure to the retrovirus or that their symptoms will be as severe or prolonged as one would expect in the absence of treatment with the antiviral agent. Without limiting the invention to methods that identify anti-viral compounds having any particular features, in certain embodiments, candidate compounds can be identified as potential anti-viral agents by virtue of their ability to bind to or modify (e.g., inhibit) the expression or activity of one or more of the host factors described herein. An antiviral compound can be a small molecule, an oligonucleotide (e.g., an antisense oligonucleotide), an siRNA, an antibody (e.g. a monoclonal antibody, a humanized antibody, a single chain antibody, or fragments thereof), or another type of protein or compound that can bind to and thereby inhibit the ability of a host factor to facilitate retroviral infection, replication, or propagation. For example, in the event the host factor is a subunit of a larger protein complex (e.g., a homodimer or heterodimer), the antiviral agent could, by virtue of binding to (or otherwise associating with) the host factor, prevent the host factor from participating in (or functioning in) the complex. The activities of many host factors are known in the art and representative examples are referenced in FIG. 5.

Antiviral agents can be identified by carrying out the methods described herein in cells in vivo or ex vivo. The cell can be a yeast cell (e.g. a Saccharomyces cell, such as S. cerevisiae), a bacterial cell (e.g., E. coli), a mammalian cell (e.g. a human cell, such as a T lymphocyte), or a cell from an established cell line. Alternatively, one can employ cell-based assays, cell fractions, cell lysates, cell extracts, or in vitro assays with partially or substantially purified host factors. Regardless of the exact configuration of the assay, the antiviral agents can be identified in a two-step process: in the first step, one identifies a compound that binds to or that inhibits the expression or activity of a host factor, and in the second step, one tests the compound for antiviral activity. For example, in one embodiment, the invention features methods of identifying antiviral agents that include the steps of: (a) exposing a host factor to a candidate compound; (b) determining whether the candidate compound binds (e.g., specifically binds) the host factor or inhibits the activity or expression of the host factor (a candidate compound that binds the host factor or inhibits the activity or expression of the host factor is a potential antiviral agent); (c) exposing a cell to the potential antiviral agent and a retrovirus; and (d) determining whether the potential antiviral agent inhibits the ability of the retrovirus to infect the cell, replicate therein, or exit the cell. A potential antiviral agent that inhibits the ability of the retrovirus to, for example, infect the cell, replicate therein, or exit the cell is an antiviral agent. The cell can be exposed to the potential antiviral agent before, during or after the cell is exposed to the retrovirus. Where the cell is a cell in vivo, one can determine whether a potential anti-viral agent is an antiviral agent by determining whether there is any improvement in a sign or symptom of the disease that is associated with the retroviral infection, or whether those signs and symptoms fail to appear as expected in the absence of administration of the antiviral agent.

The host factor can be partially or substantially pure (e.g. it can be separated from some or substantially all of the materials with which it is naturally associated; e.g., 50, 60, 70, 75, 80, 85, 90, 95, 98, 99, or 100% pure) or in, for example, a cell fraction, lysate, or extract. In these methods and other embodiments, in addition to determining, or as an alternative to determining, in step (b), whether the candidate compound binds (and, preferably, specifically binds) the host factor, one can determine whether the candidate compound inhibits the ability of the host factor to function. For example, one can determine whether the candidate compounds inhibit one or more of the activities of the host factor (again, some of these are noted in Table 2 and referenced further in FIG. 5) or the host factor's expression.

As noted above, the methods of the invention can be carried out using intact or whole cells. Accordingly, the invention features methods for identifying an antiviral agent by: (a) exposing a first cell that expresses a host factor to a candidate compound; (b) determining whether the candidate compound binds to the host factor or inhibits the expression or activity of the host factor in the first cell (a candidate compound that inhibits the expression or activity of the host factor in the first cell is a potential antiviral agent); (c) exposing a second cell to the potential antiviral agent and a retrovirus; and (d) determining whether the potential antiviral compound inhibits the ability of the retro virus to, for example, infect or replicate within the second cell. A potential antiviral compound that inhibits the ability of the retrovirus to infect or replicate within the second cell is an antiviral compound. As described further below, the first cell and the second cell (as referenced in any of the methods of the invention) may be of the same type or of different types and; if one desires, the first cell and the second cell may be the same cell.

The gene encoding a host factor can be deleted or inhibited in non-yeast cells (e.g. a mammalian cell, such as a primary human cell or a cell from an established human cell line) by any method known in the art (e.g., gene deletion or RNAi). That cell, or cells derived from the initial deletant cell, are within the scope of the present invention. Such cells (which can be isolated or placed in culture) can be used to determine whether the gene that was deleted (or otherwise inhibited) encodes a protein that facilitates retroviral infection or replication. It does so if, in its absence, a given retrovirus is less able to infect or replicate within the cell. Accordingly, the invention also features methods of determining whether a host factor is a promising target for a therapeutic agent. These methods can be carried out, for example, by exposing a cell in which one or more host factors have been silenced or impaired (by a knock out, other mutation, or antisense or RNAi procedure) to a retrovirus. Such a cell is exposed to a retrovirus under conditions that would allow the retrovirus to infect the cell and carry out its life cycle. If the host factor is a promising target for a therapeutic agent, the retrovirus will not infect the cell or complete its life cycle as successfully as it otherwise would (control experiments using, for example, a corresponding wildtype cell, can be carried out). Any of the host factors described herein can be used in such an assay and any of the reagents suitable for use in the screening assay described above are suitable for use in identifying promising drug targets. For example, one can examine yeast or human host factors and either (or both in combination) can be studied in yeast or human cells. This method can be carried out before one screens for antiviral agents per se.

Preferably, the cell (be it the first, second, or only cell used) is one that is naturally infected by a retrovirus, but it can also be a cell that is rendered susceptible to infection (by, for example, being made to express appropriate receptors for the virus in question).

In the various embodiments of the invention, the host factor can be a yeast or human host factor or, where more than one factor is present, a combination thereof. Alternatively, the host factor can be a homologous protein from another species or, as described above, a fragment, other mutant, or variant of any of these proteins. The factor(s) can be naturally expressed by a cell employed in the assays described herein or they can be expressed following transfection with an appropriate nucleic acid sequence (optionally, under the control of a constitutively active or inducible promoter and/or other regulatory elements). Cells that have been genetically modified to express a host; factor are also within the scope of the invention. The nucleic acid sequence can also encode an affinity tag to facilitate purification or to confer some other desirable attribute. In the event the host factor is a human host factor, it can include the sequence of any of SEQ ID NOs:5-501.

Kits containing reagents to carry out the methods of the invention and those reagents per se are also within the scope of the present invention. For example, the invention features collections of the host factors described herein (yeast and human) and nucleic acid sequences encoding them. For example, the invention features a kit that includes the yeast host factor Ard1 and/or Nat1, Sin3, or Spt4, or one or more of the corresponding human homologs and one or more of the reagents necessary for determining whether the host factor(s) included retain their biological activity in the presence of a candidate anti-retroviral agent (e.g. a protein substrate to assess acetyltransferase or deacetylase activity). The same kit could include the DNA repair protein Rad52 and reagents that could be used to examine the ability of this host factor or a homologue or derivative thereof, to mediate homologous recombination in the presence of a candidate antiviral agent. Alternatively, or in addition, the kit can contain a host factor that influences protein folding or otherwise modifies cellular proteins (e.g., kinases and proteases) and reagents for assaying these biological activities. These descriptions exemplify the kits of the invention. Others may contain any combination of the yeast or human host factors we identified (the yeast host factors are shown in Tables 1 and 2 and the human homologues are shown in FIG. 4). The factors, or cells that express them, and reagents to assay their expression or activity (i.e., an activity set out in Table 2 or FIG. 5) in the presence of candidate antiviral agents, can be packaged with instructions for use (which may be written or contained in some other medium).

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1A and 1B illustrate events relevant to the functional genomic screen we used to identify genes that affect Ty1. FIG. 1A is a schematic of the test Ty1 plasmid pAR100 (a composition within the scope of the invention), which was introduced into each of the 4,483 S. cerevisiae deletion strains cstested.

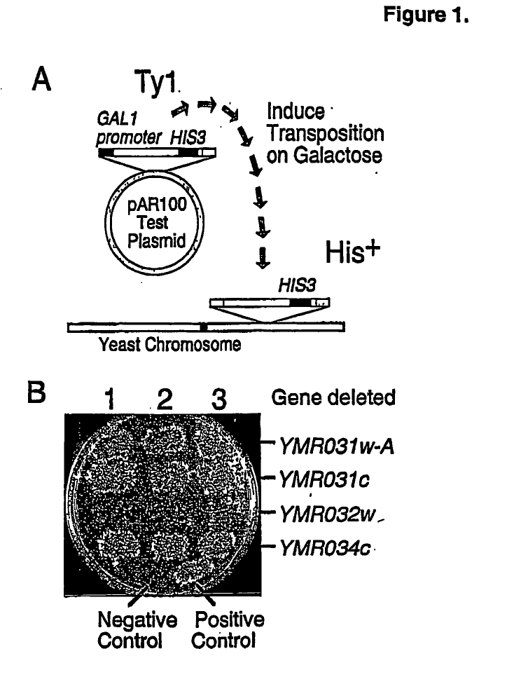

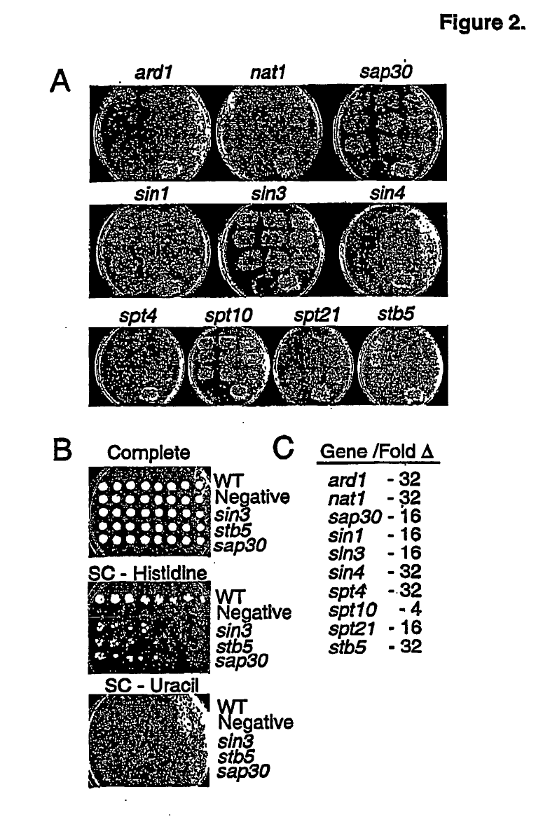

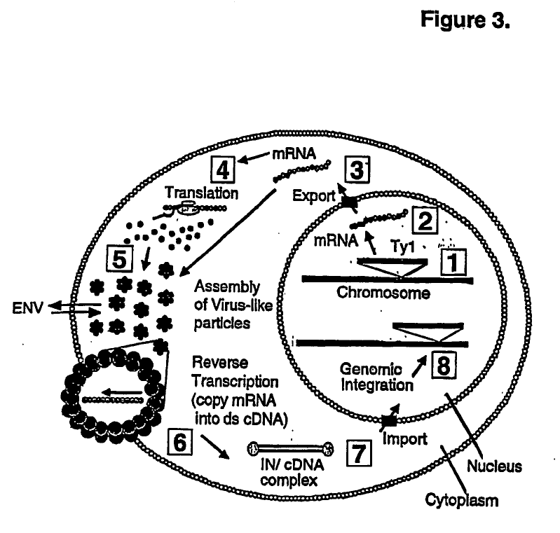

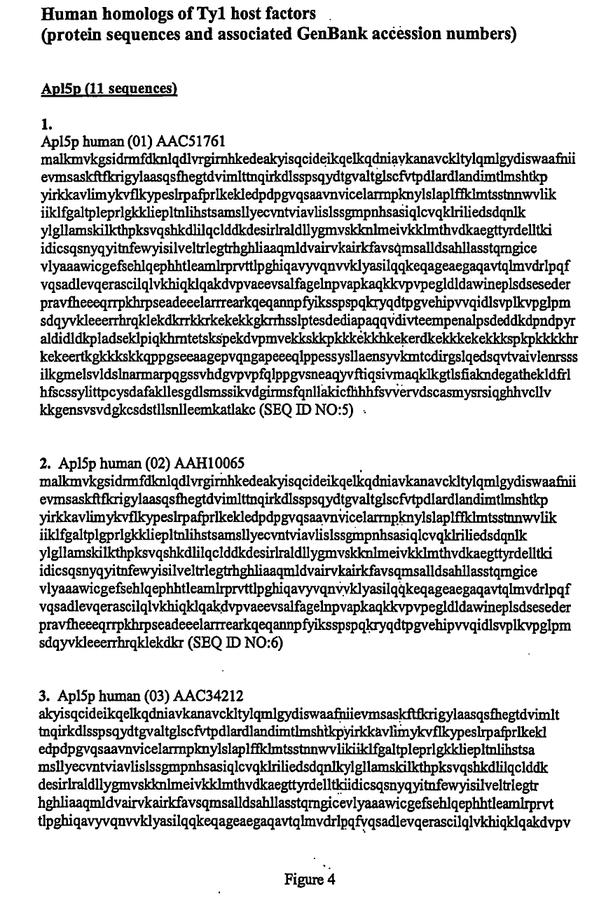

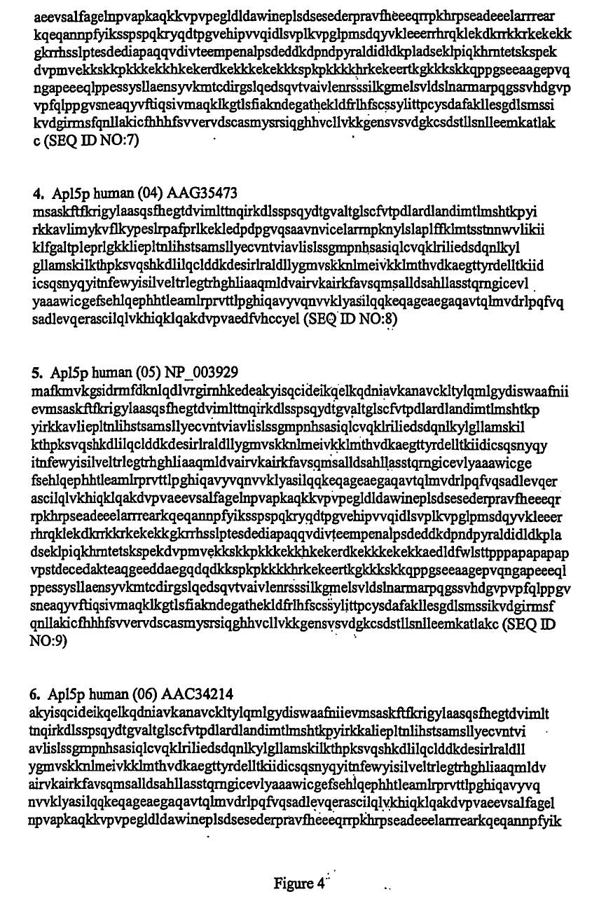

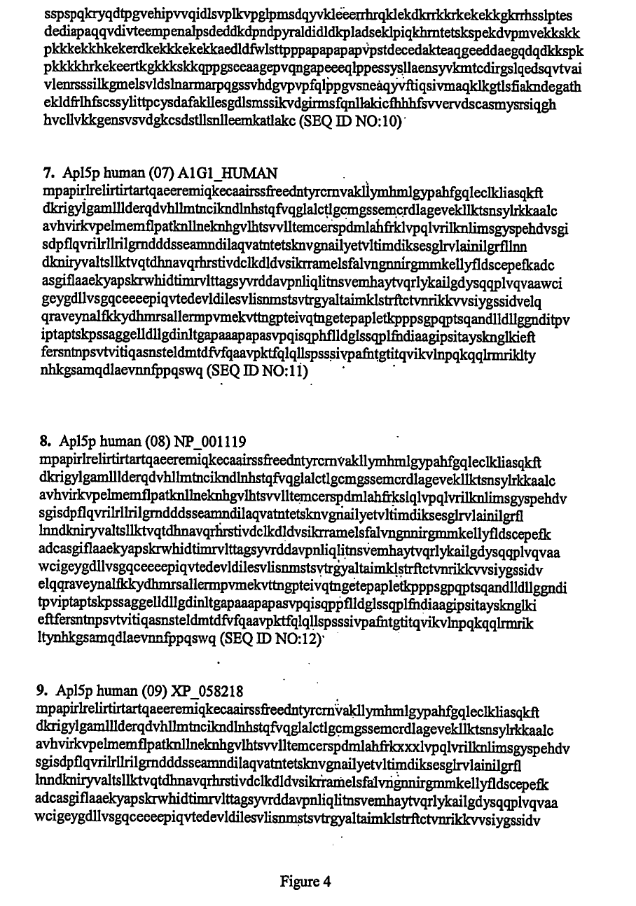

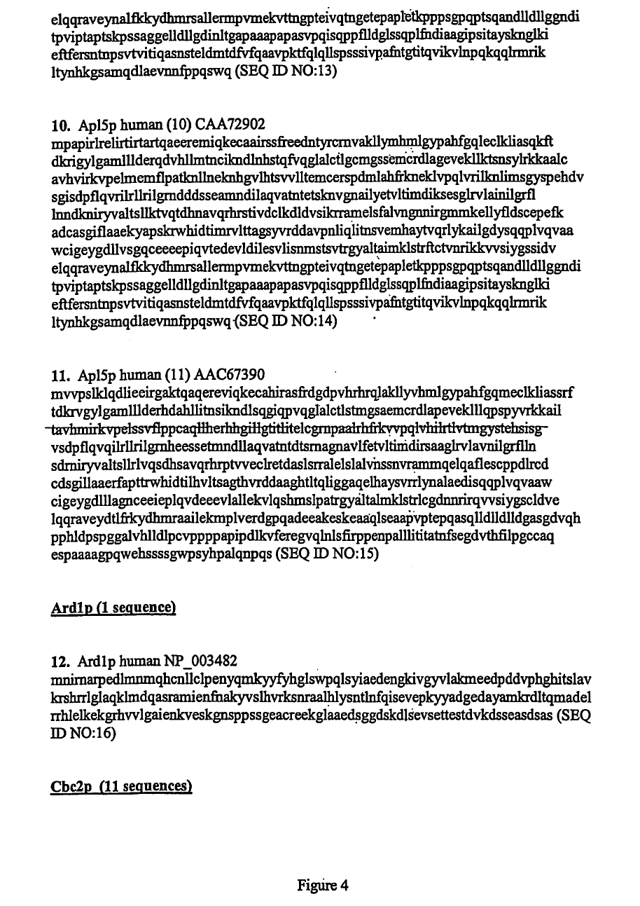

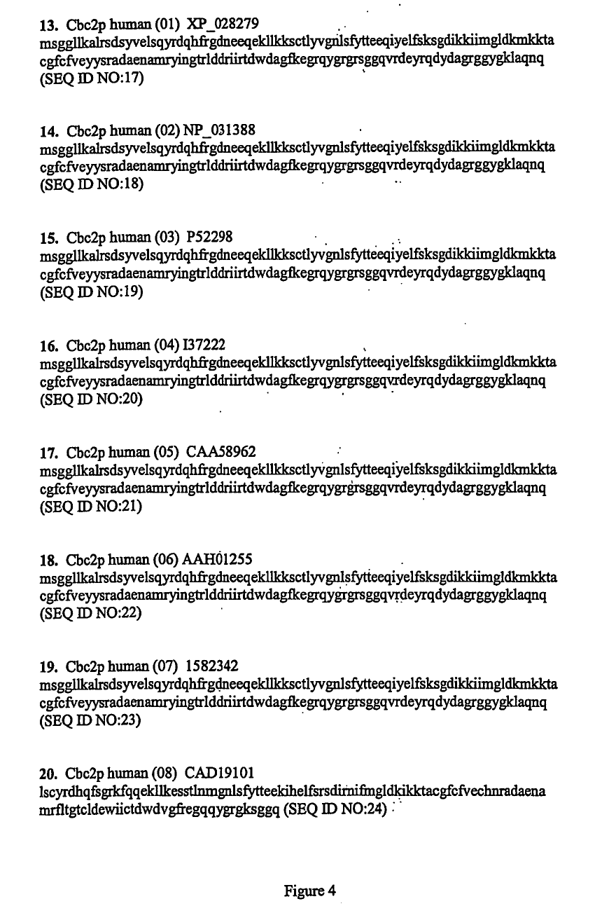

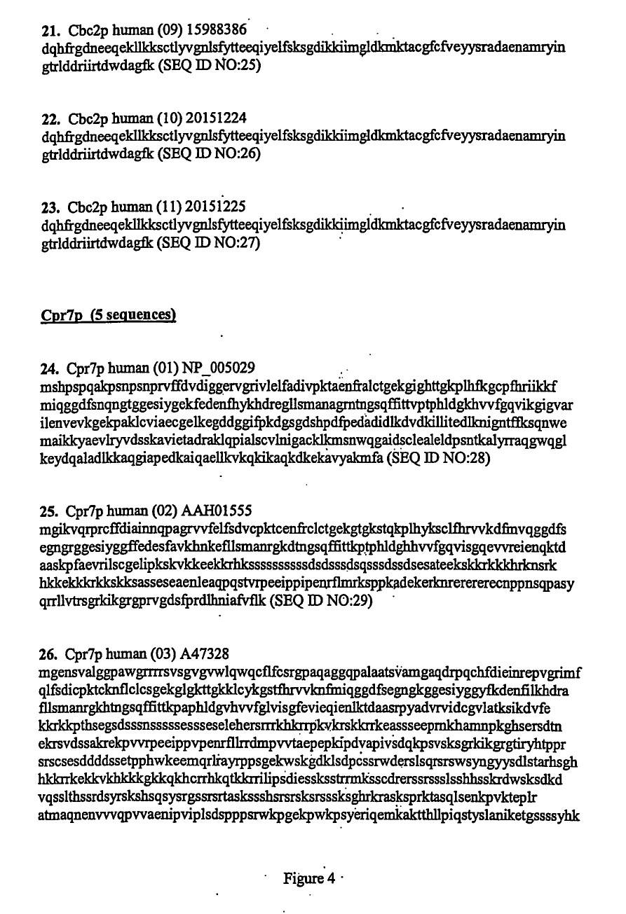

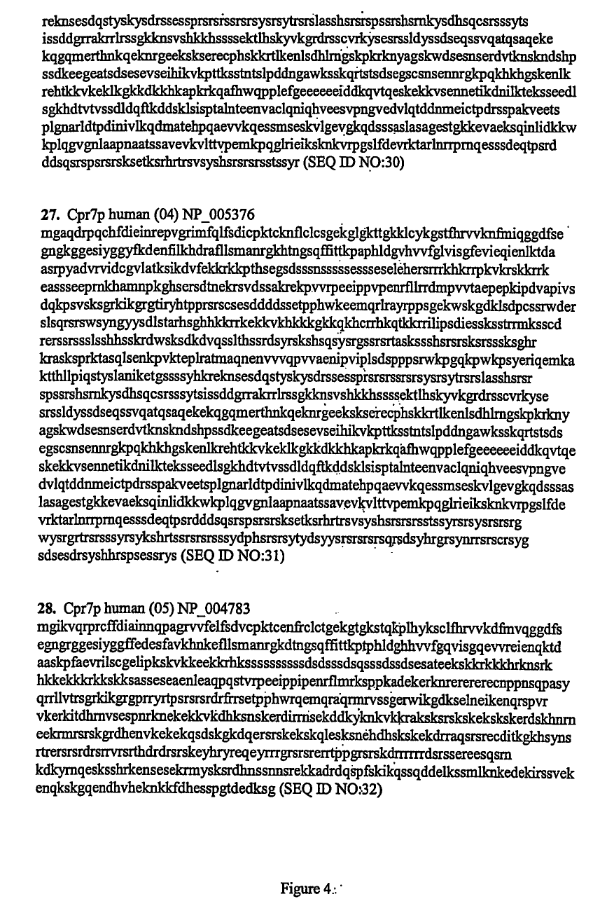

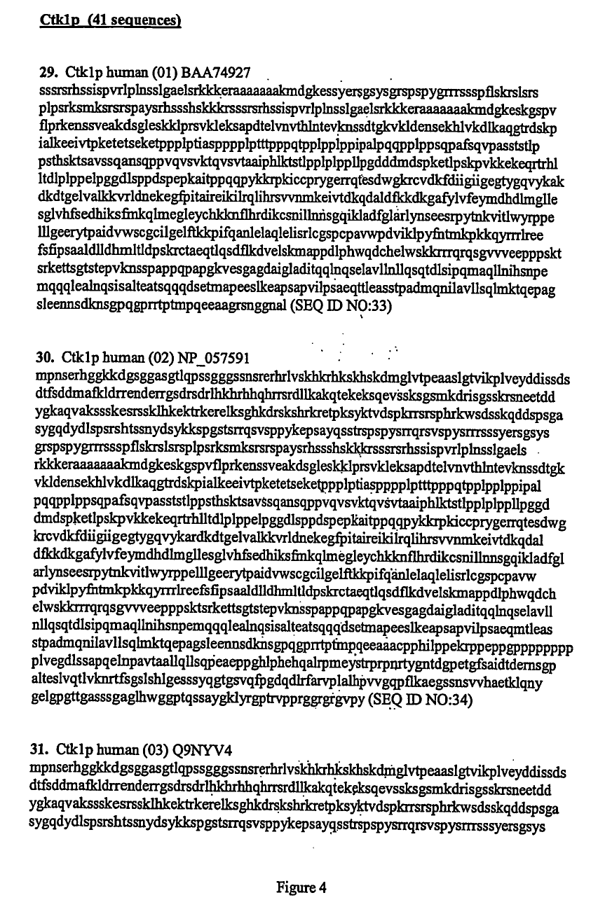

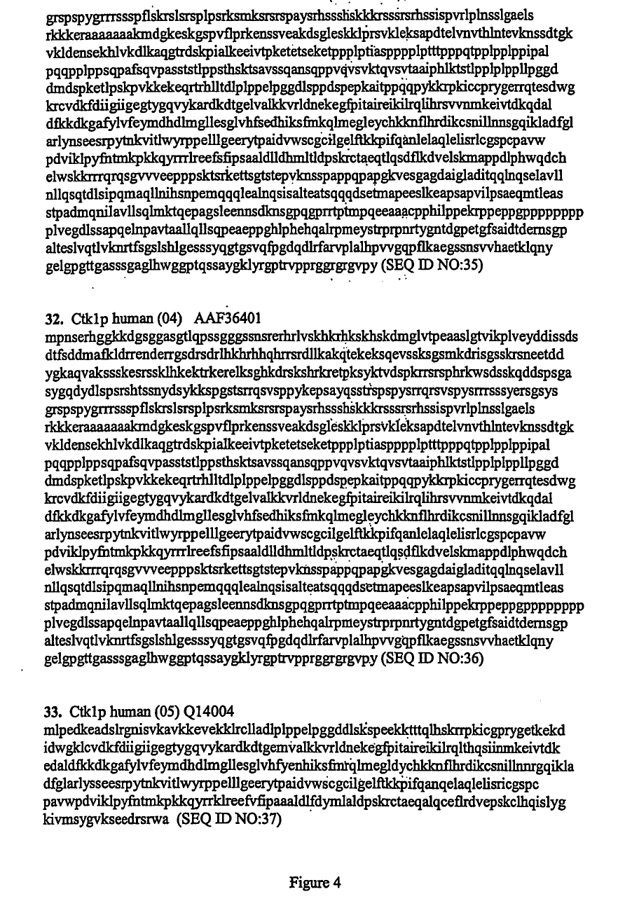

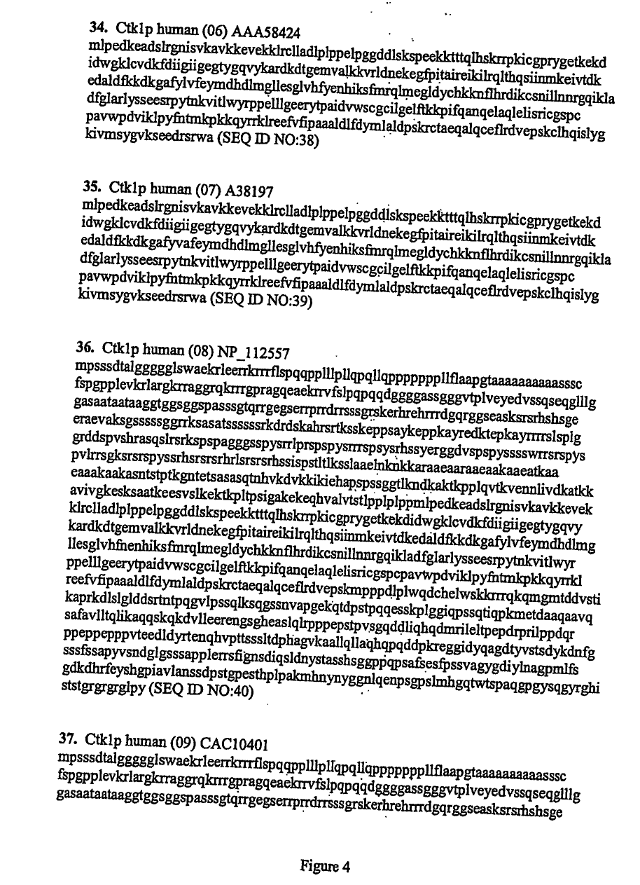

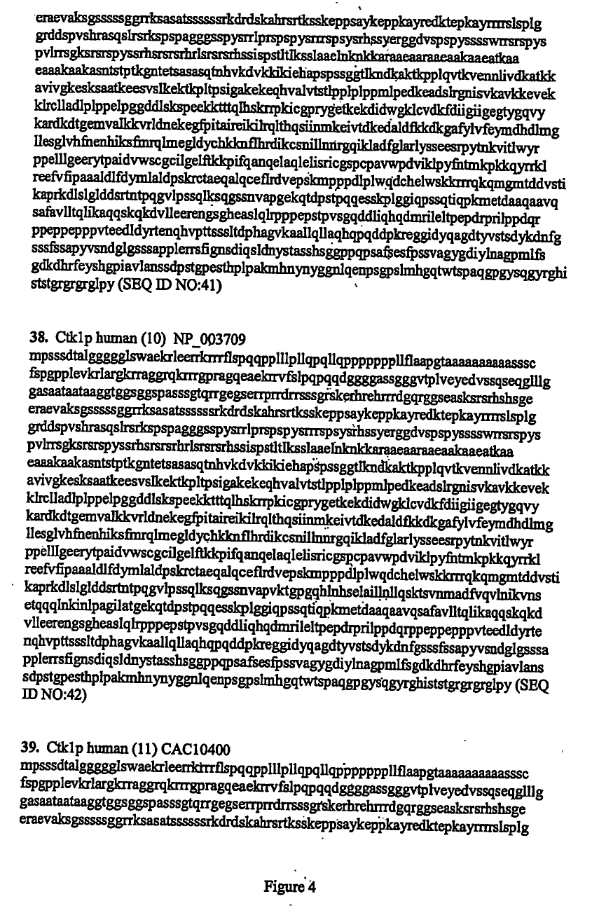

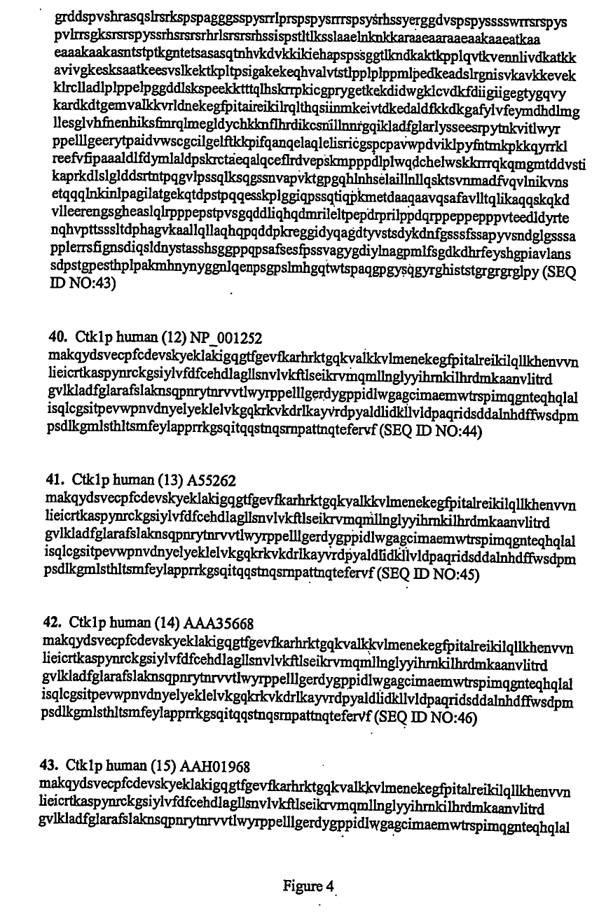

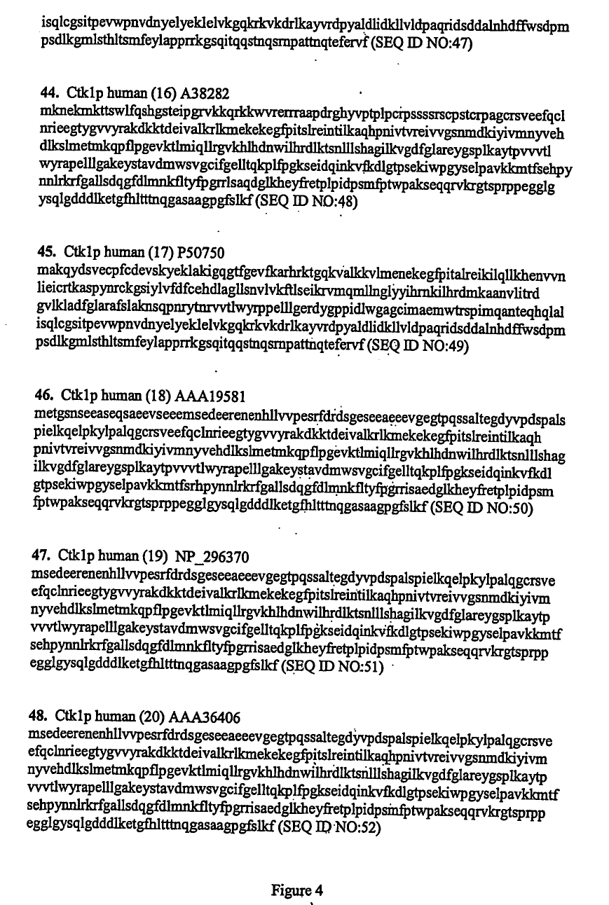

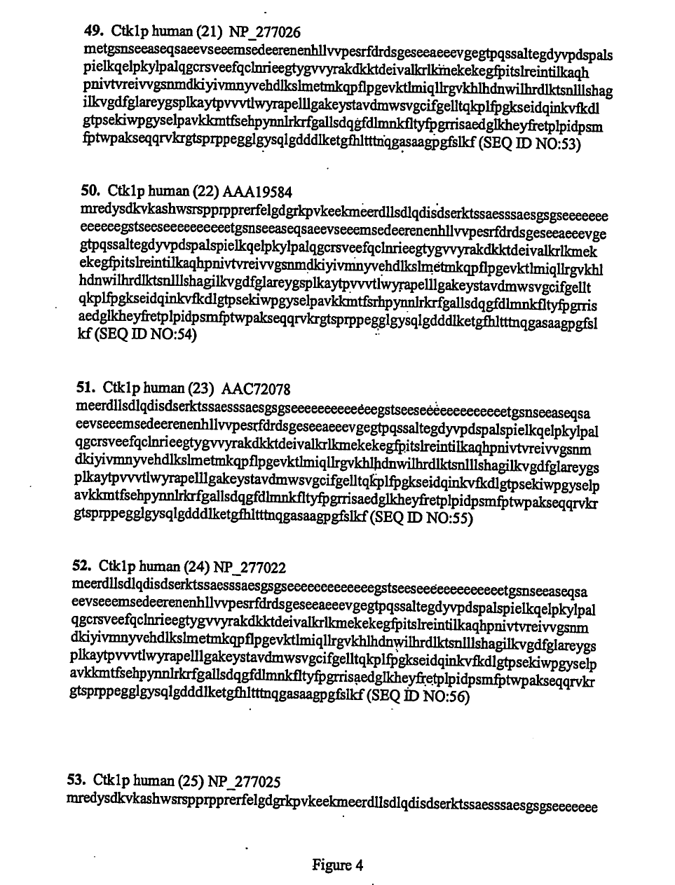

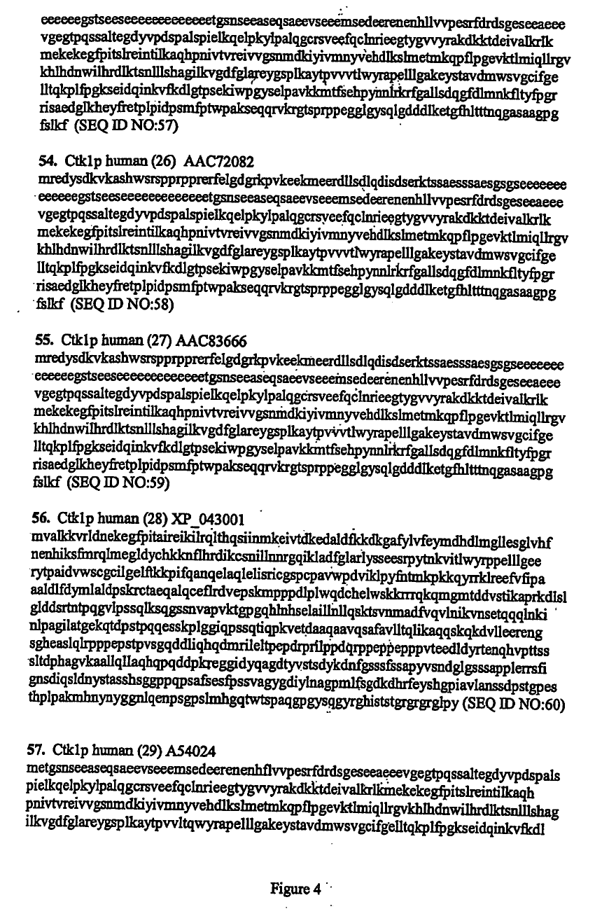

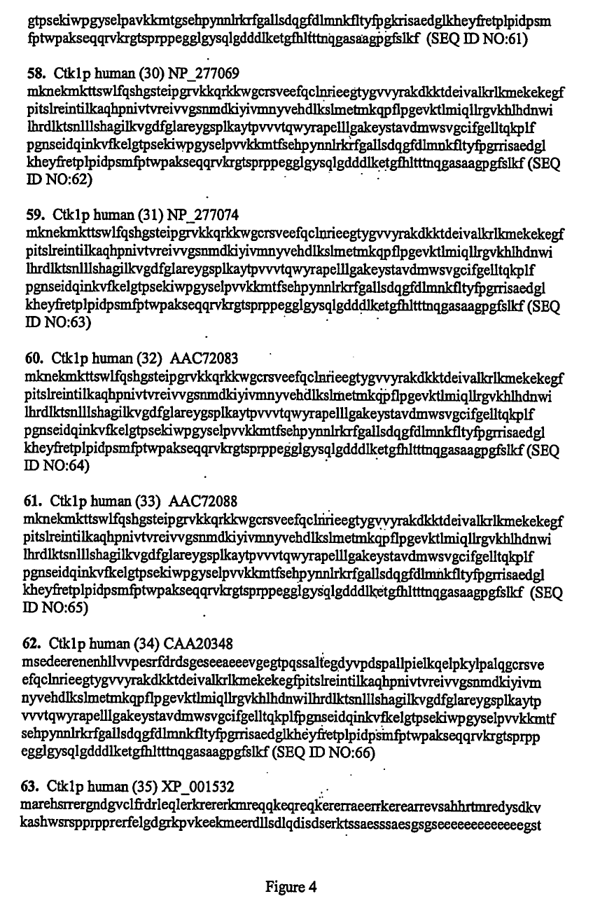

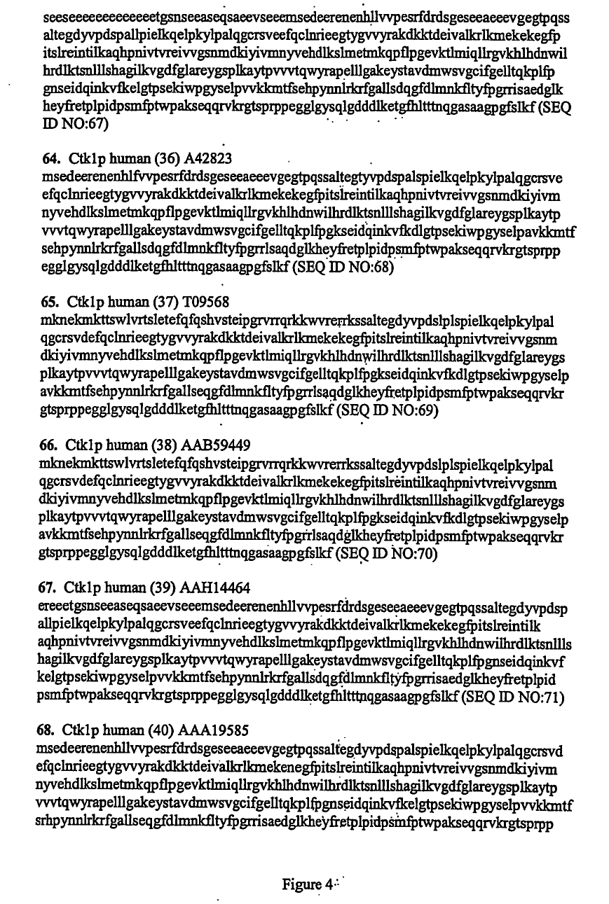

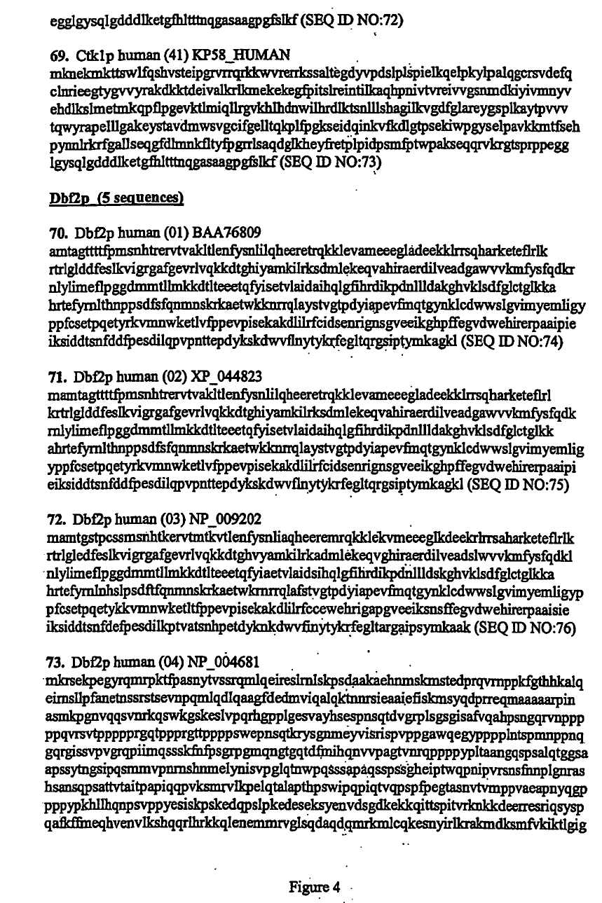

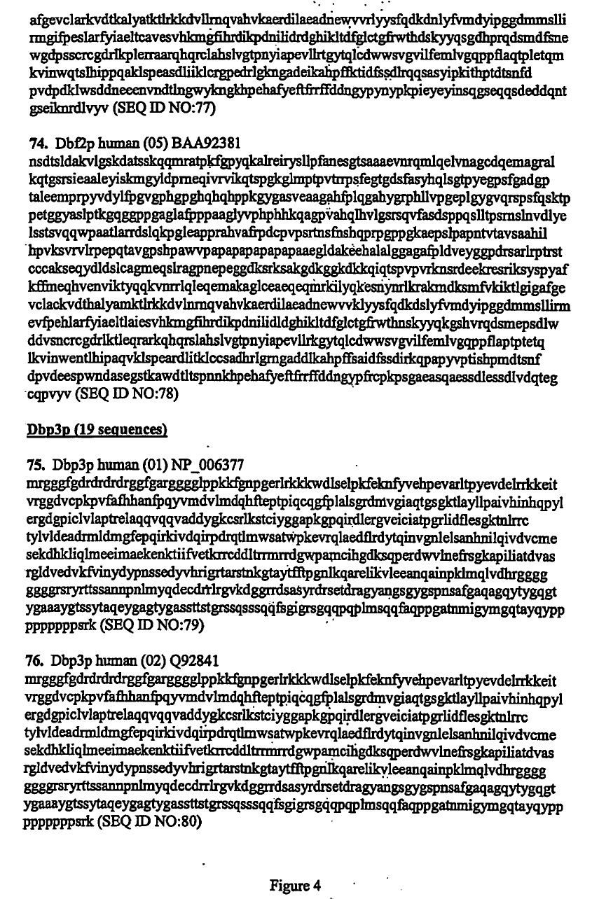

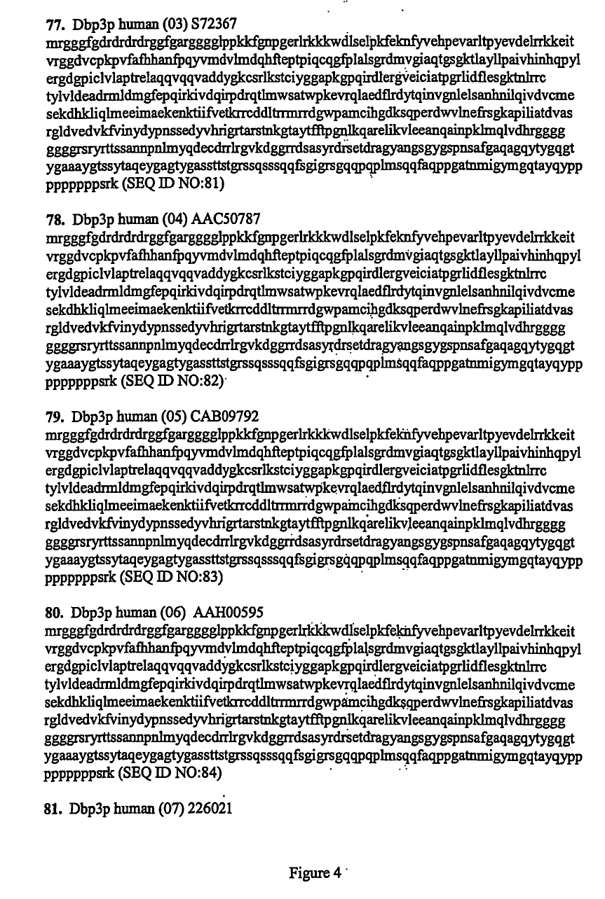

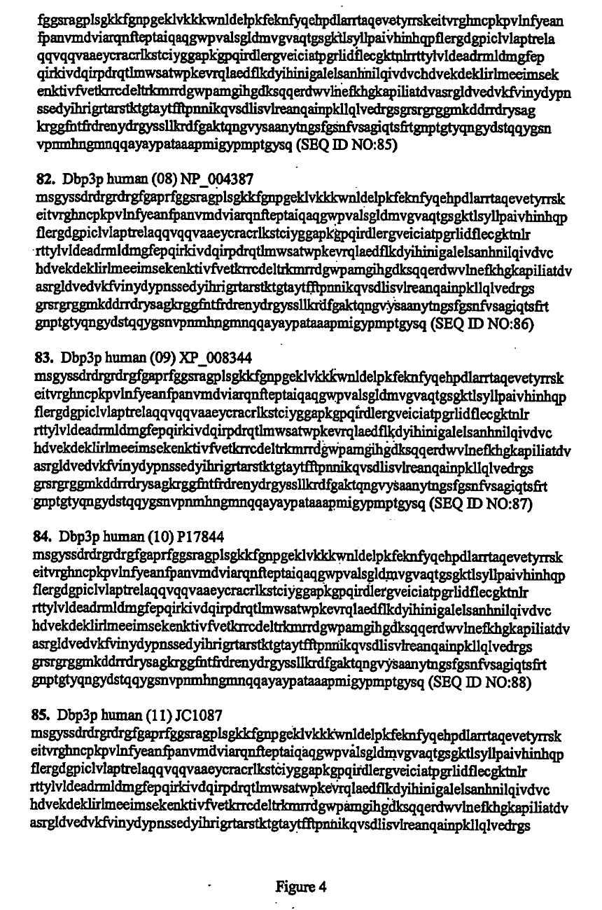

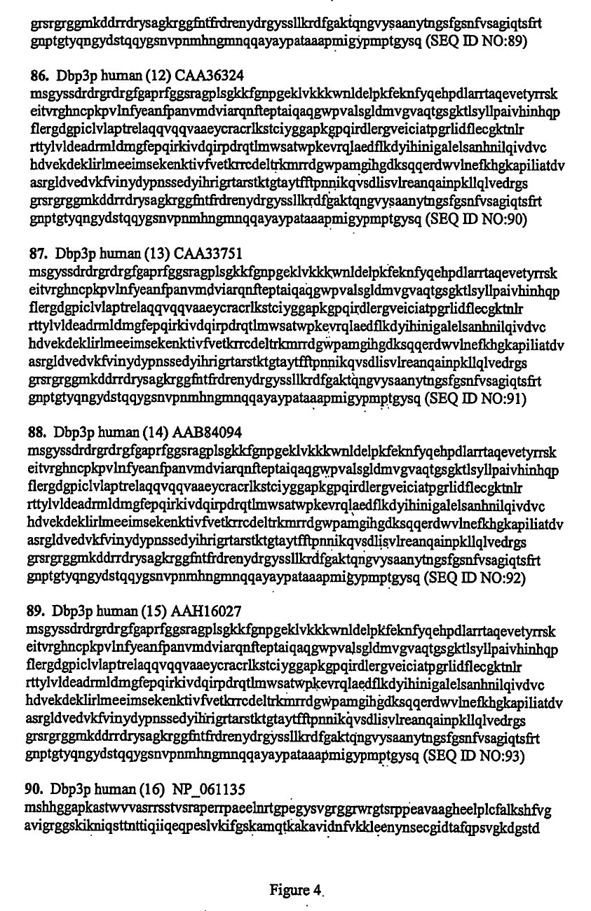

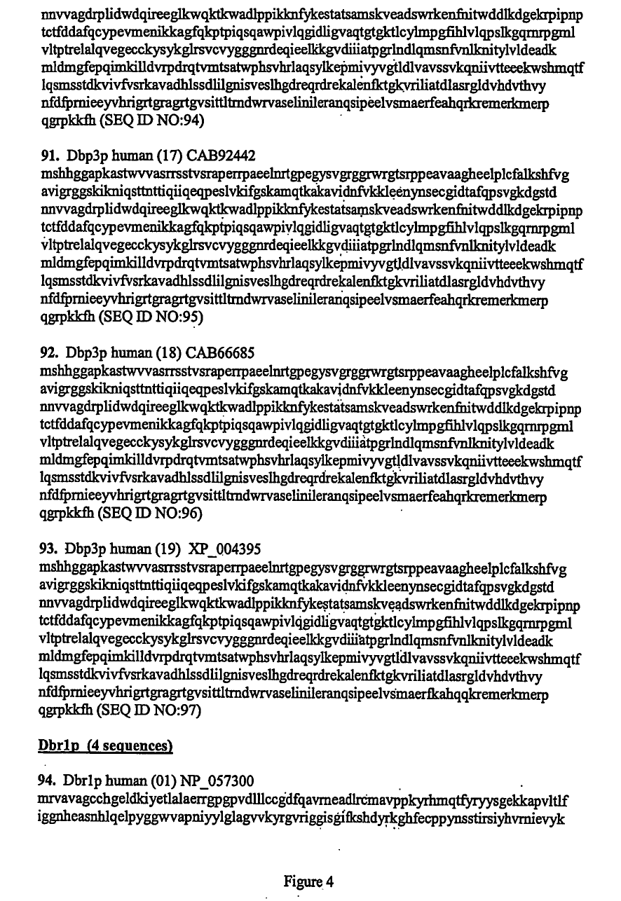

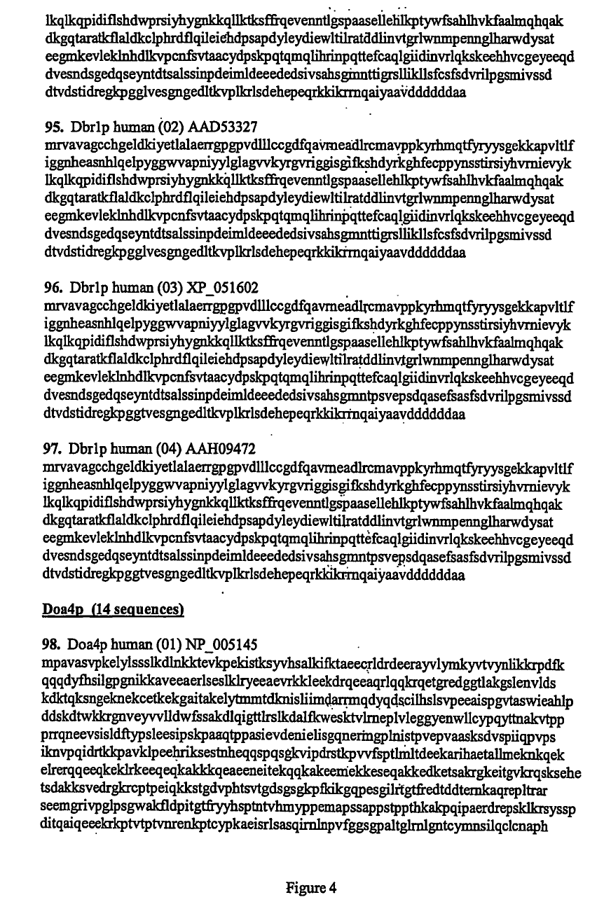

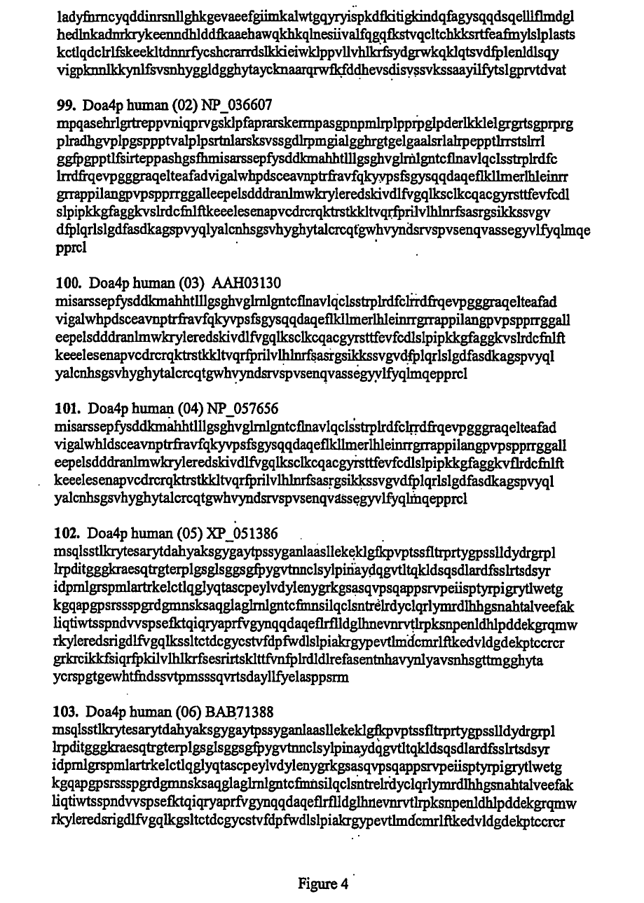

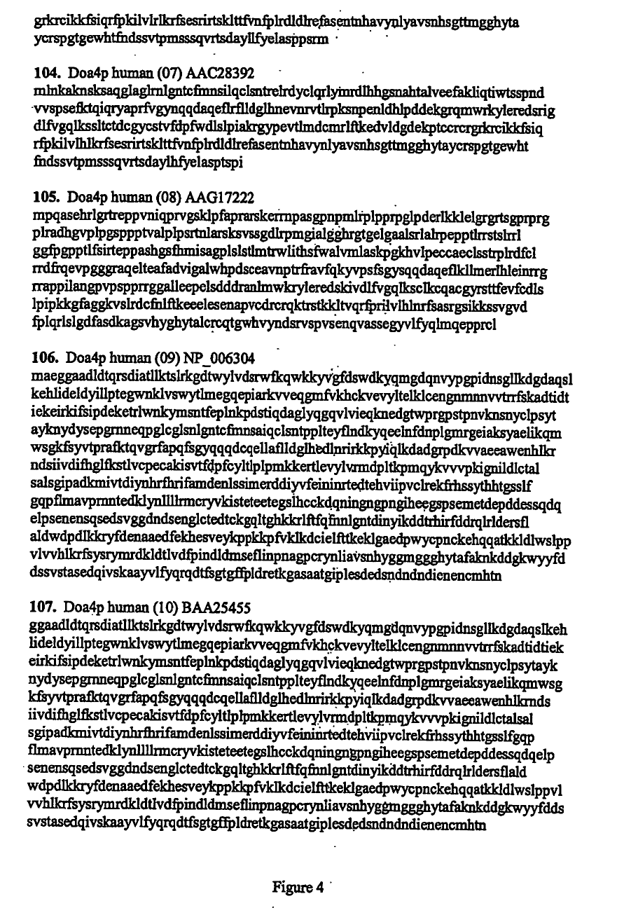

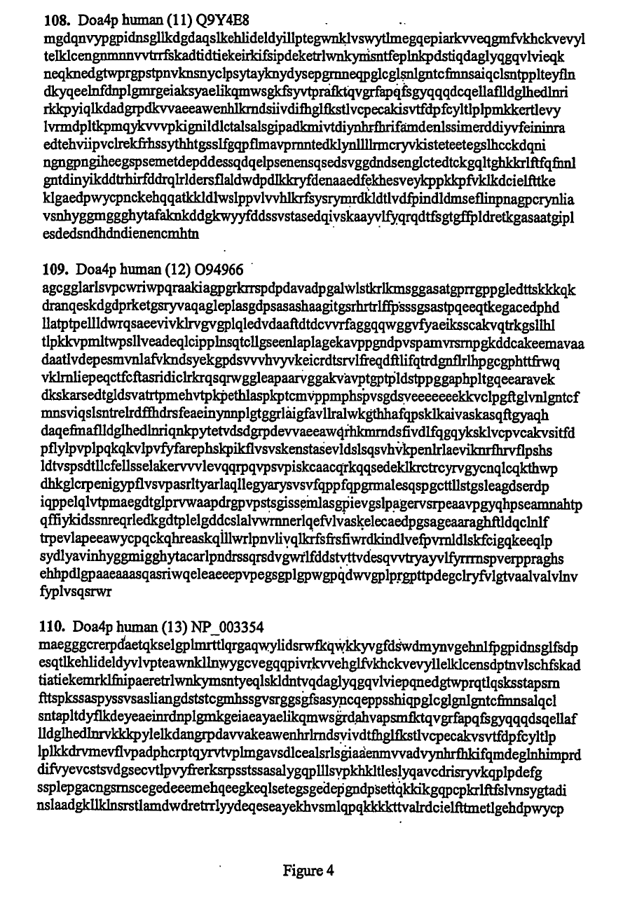

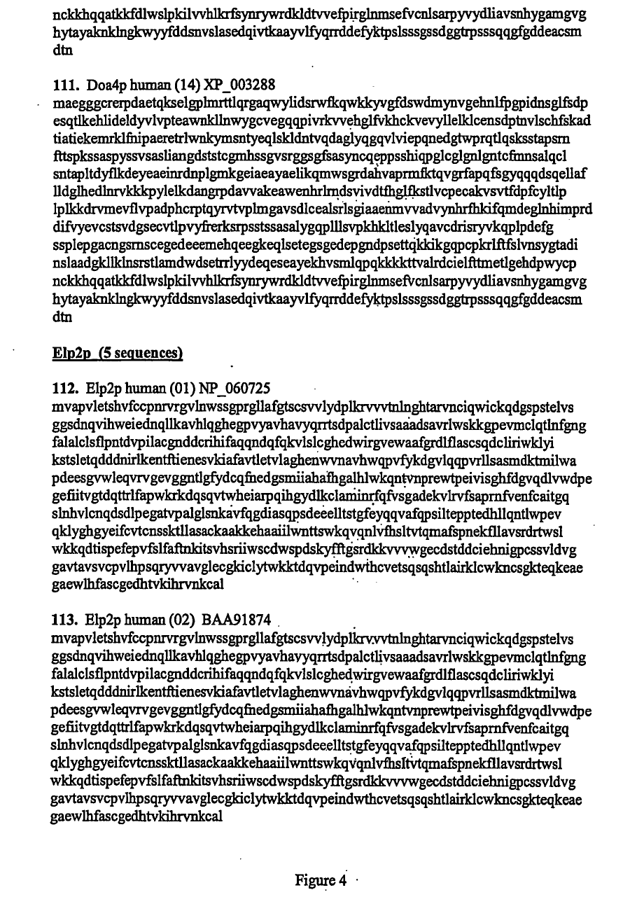

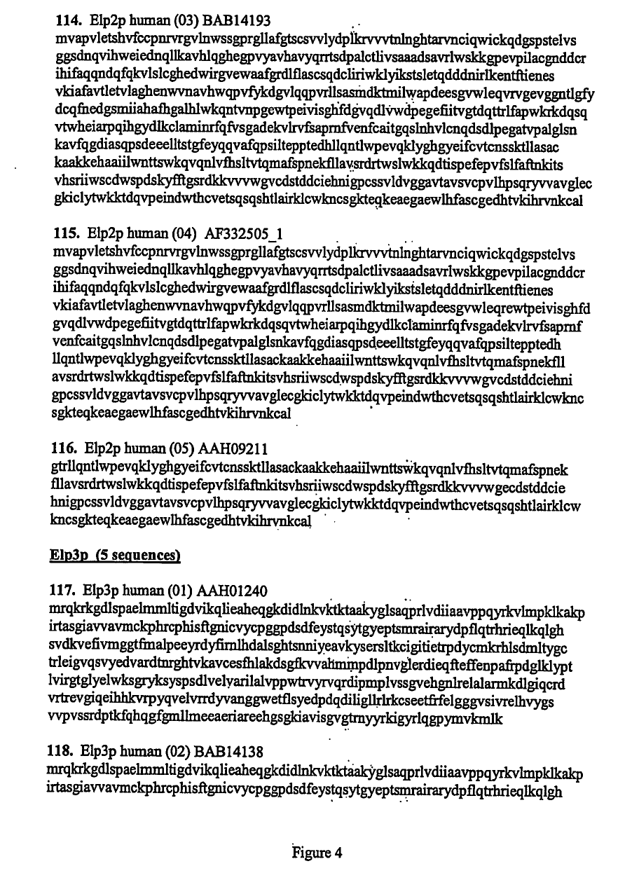

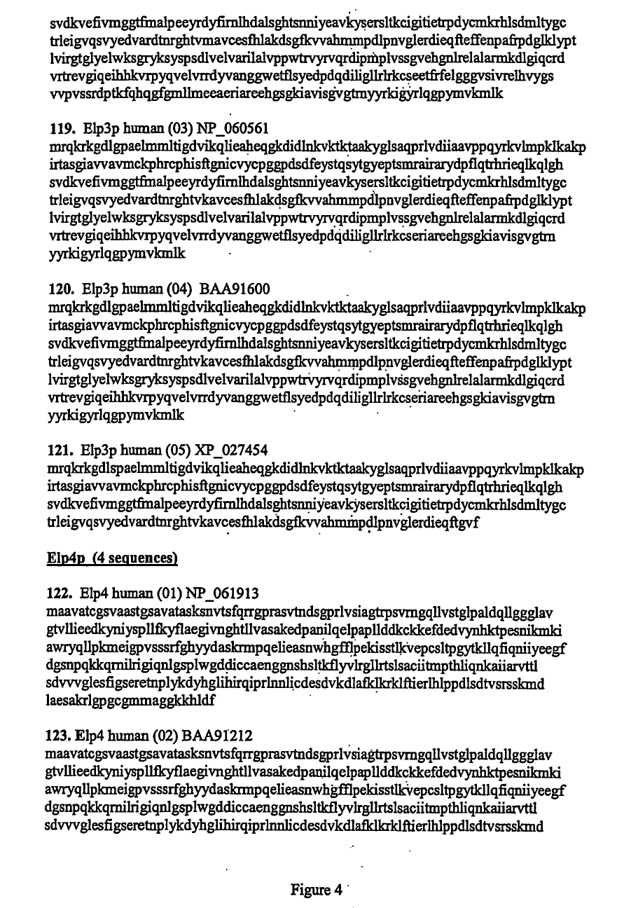

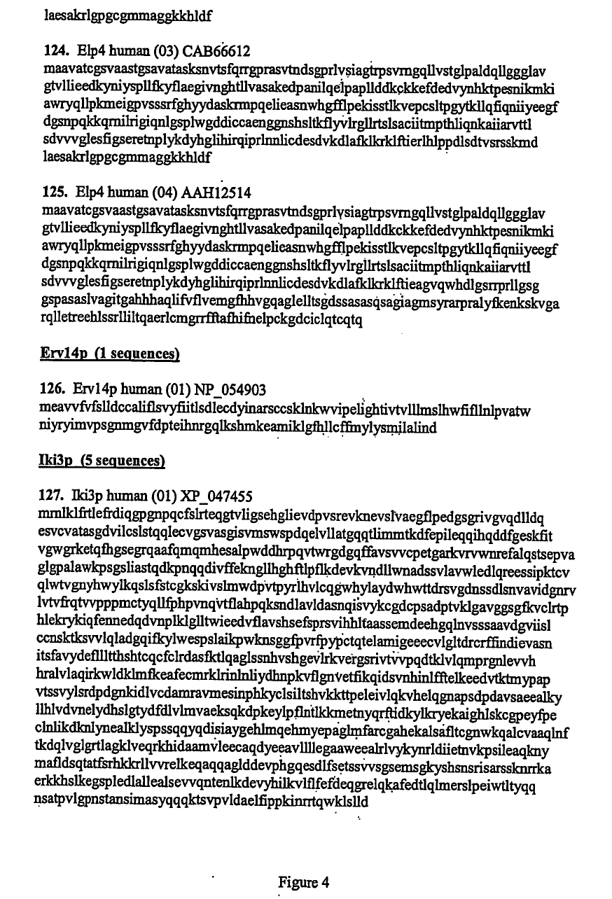

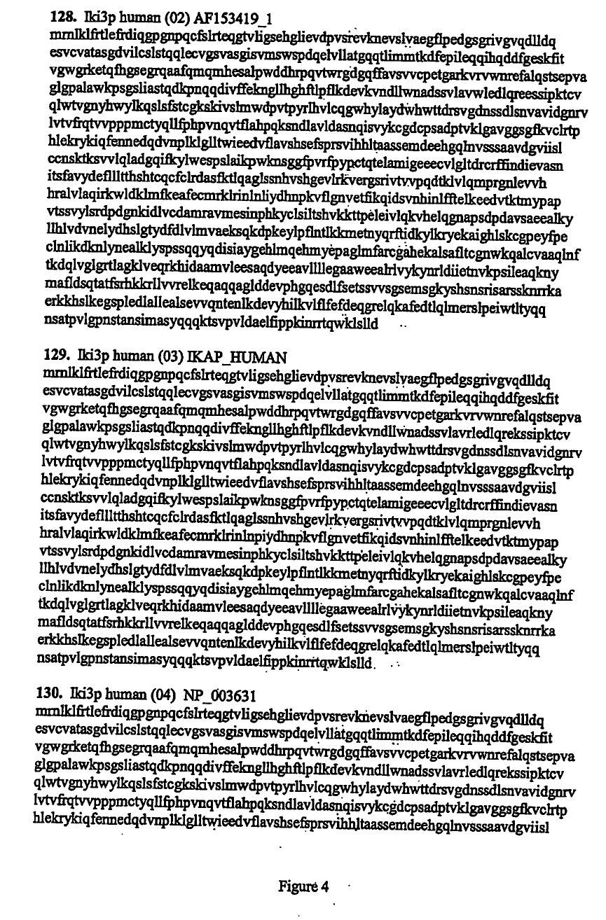

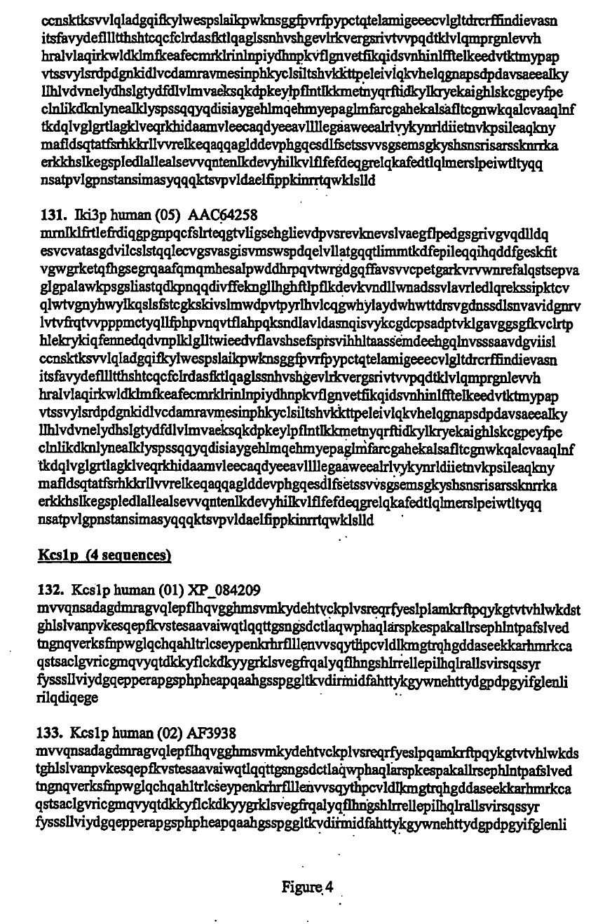

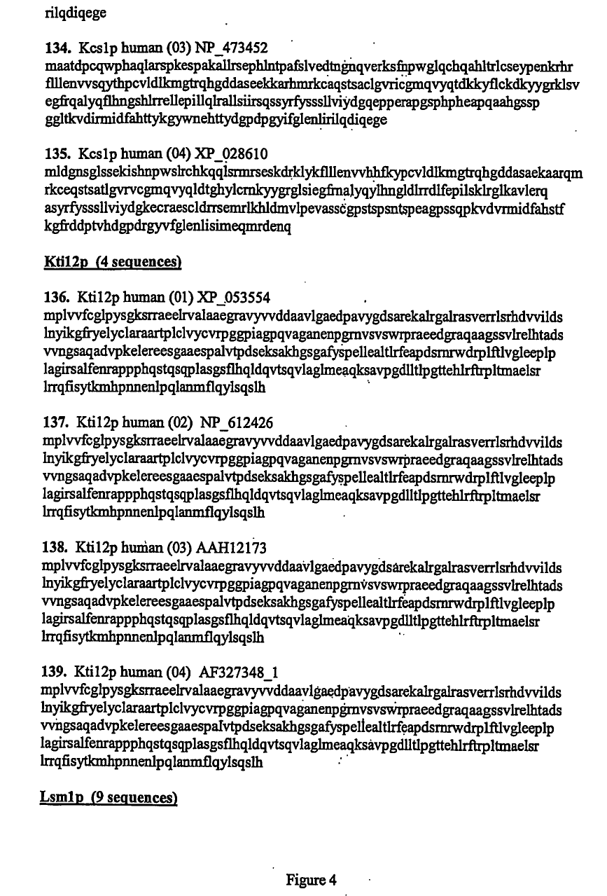

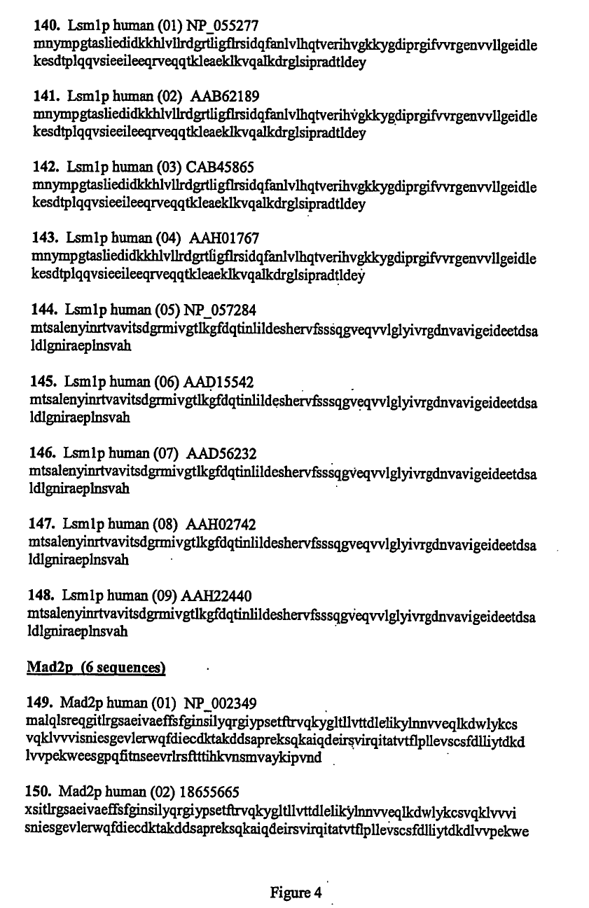

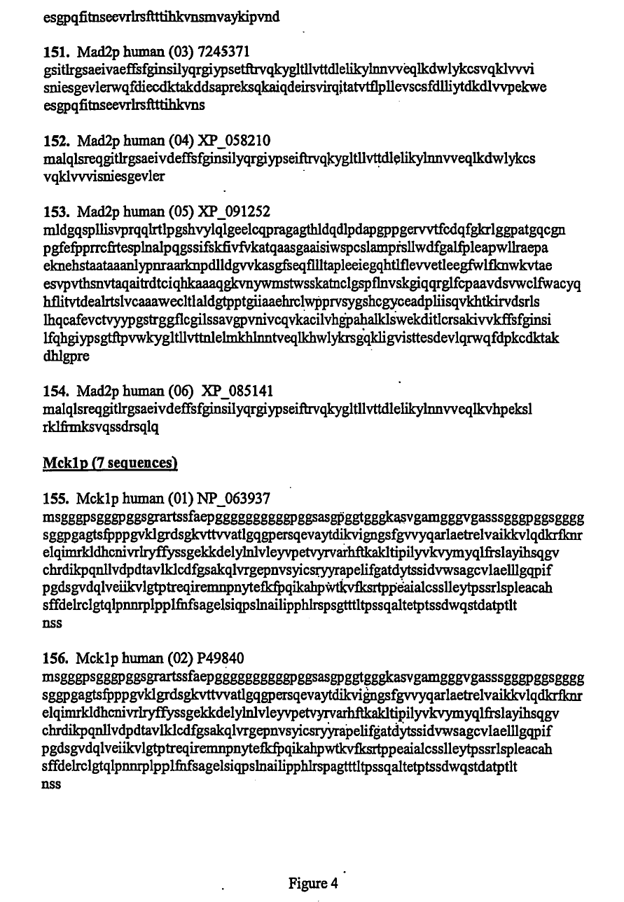

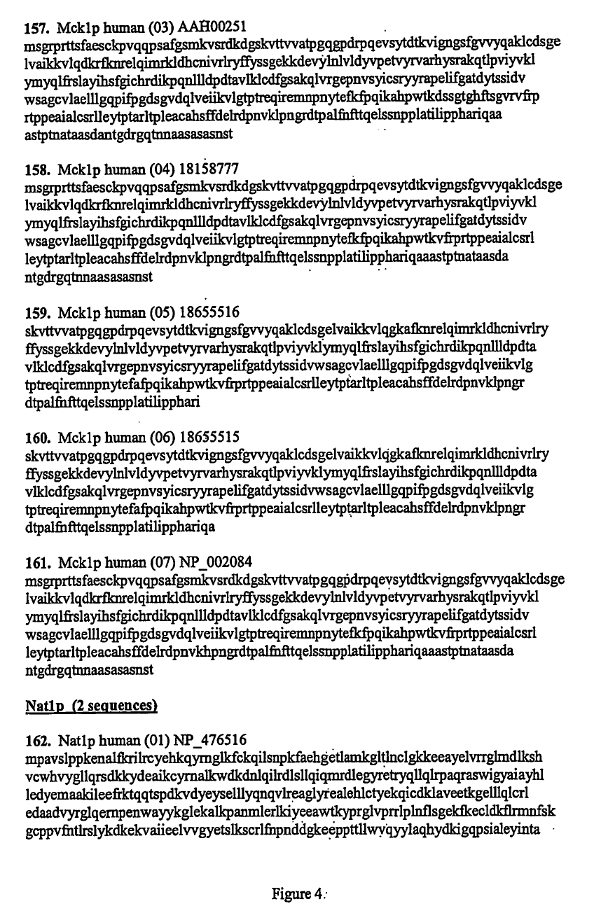

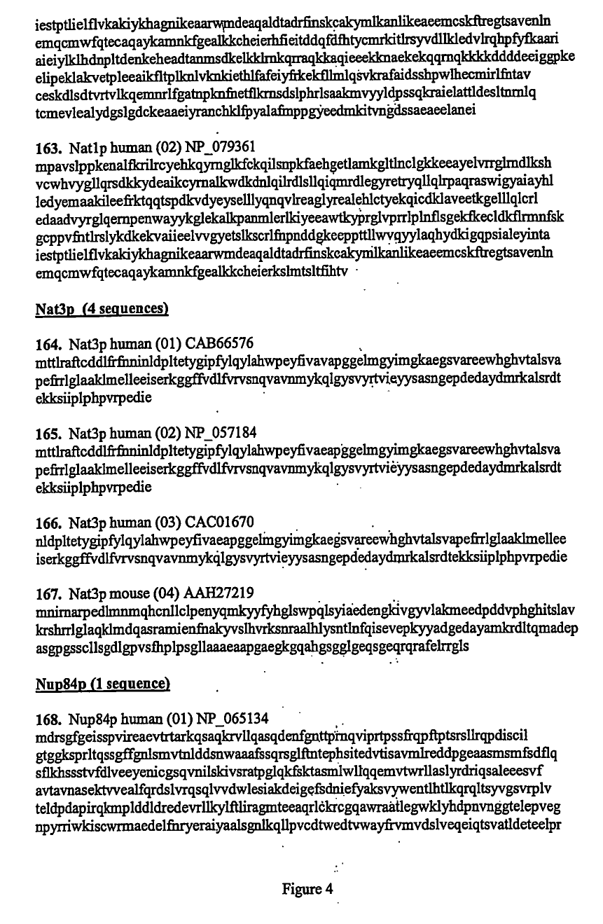

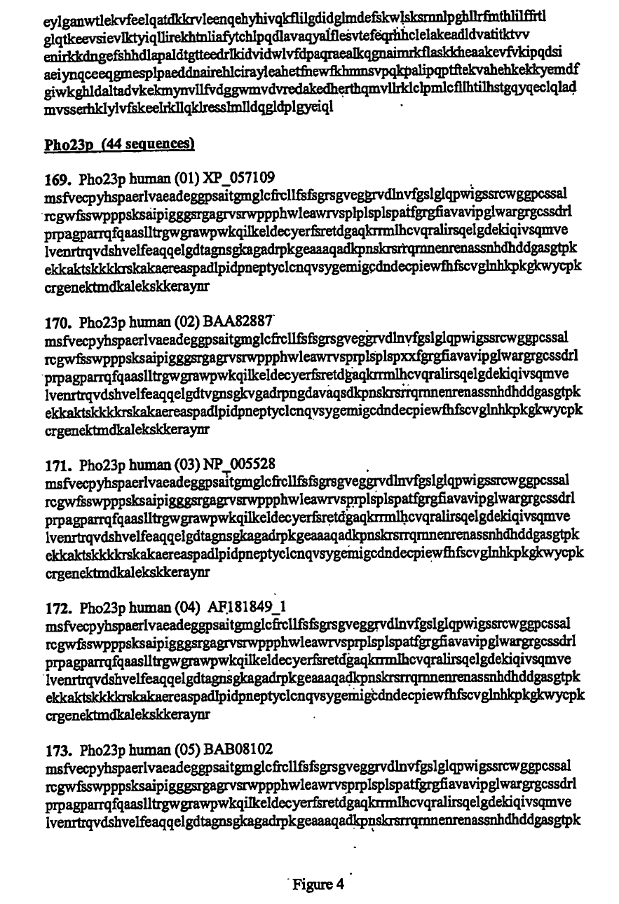

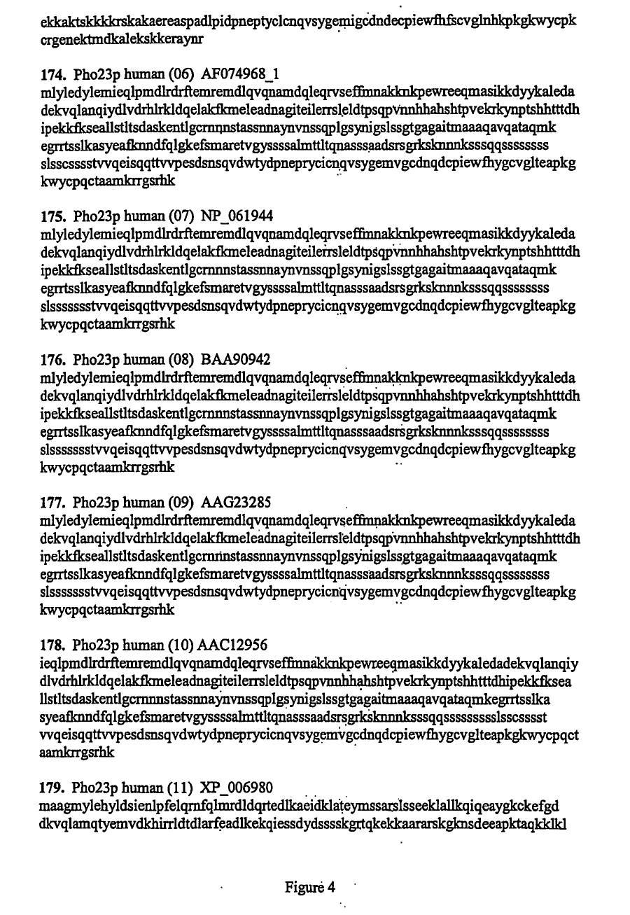

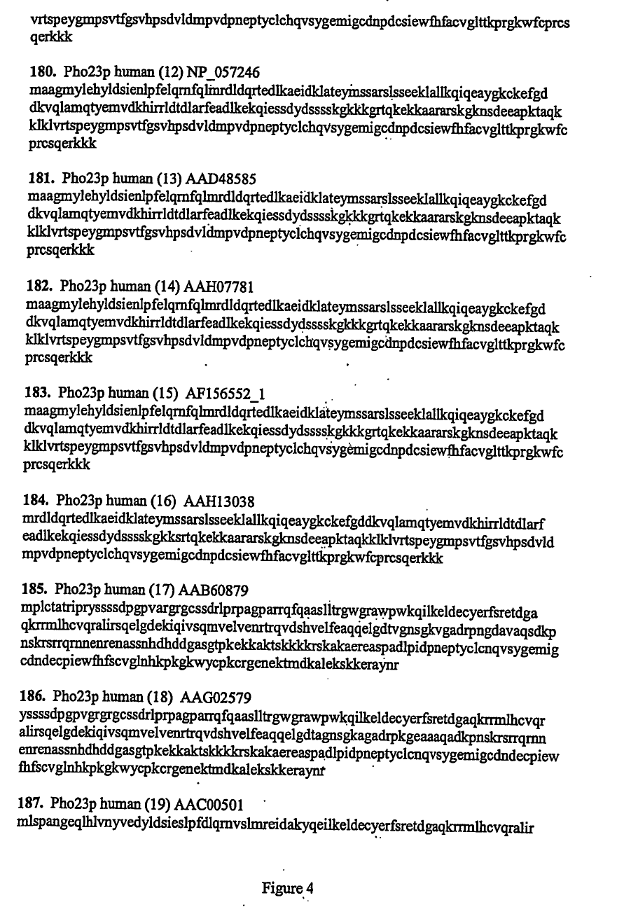

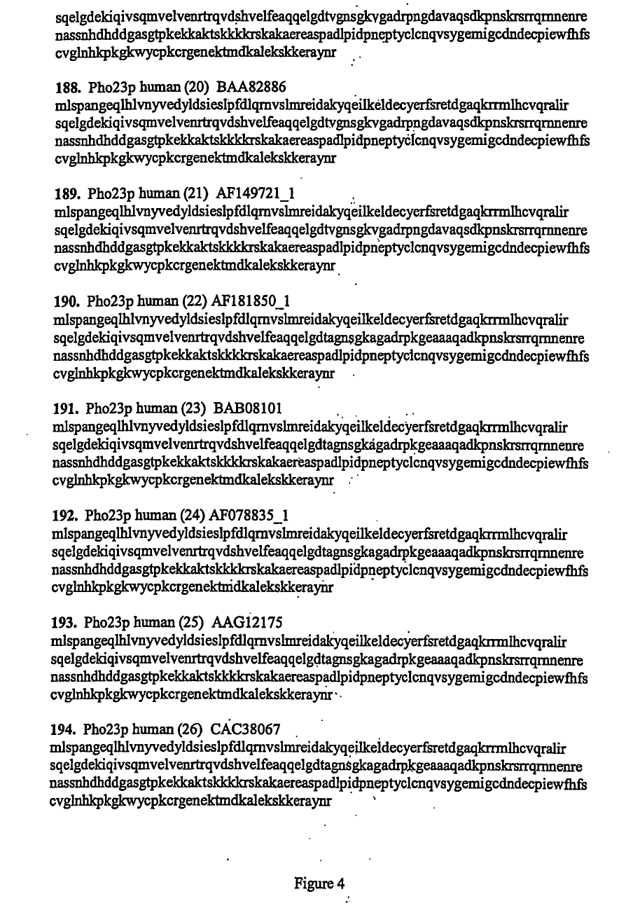

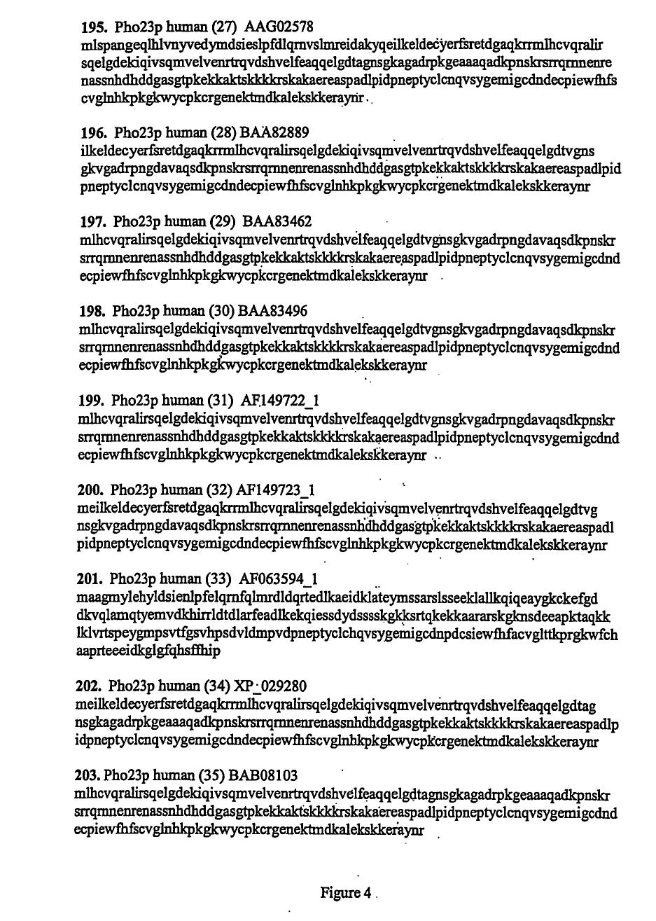

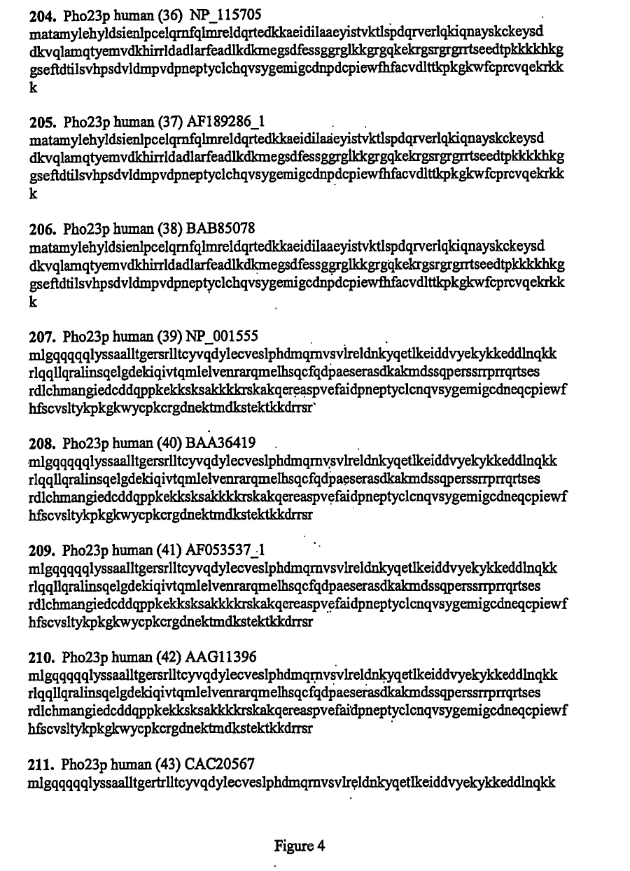

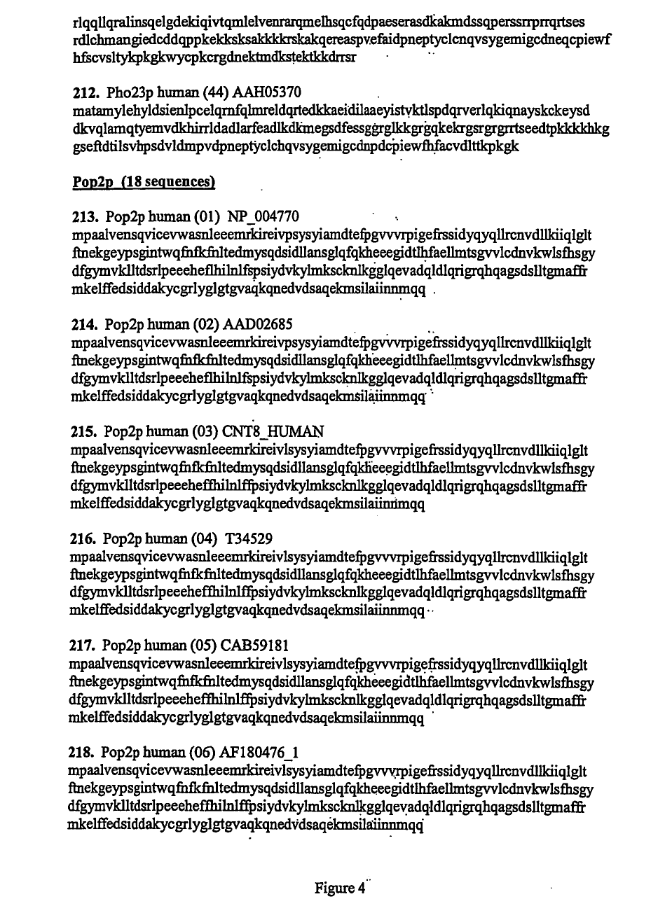

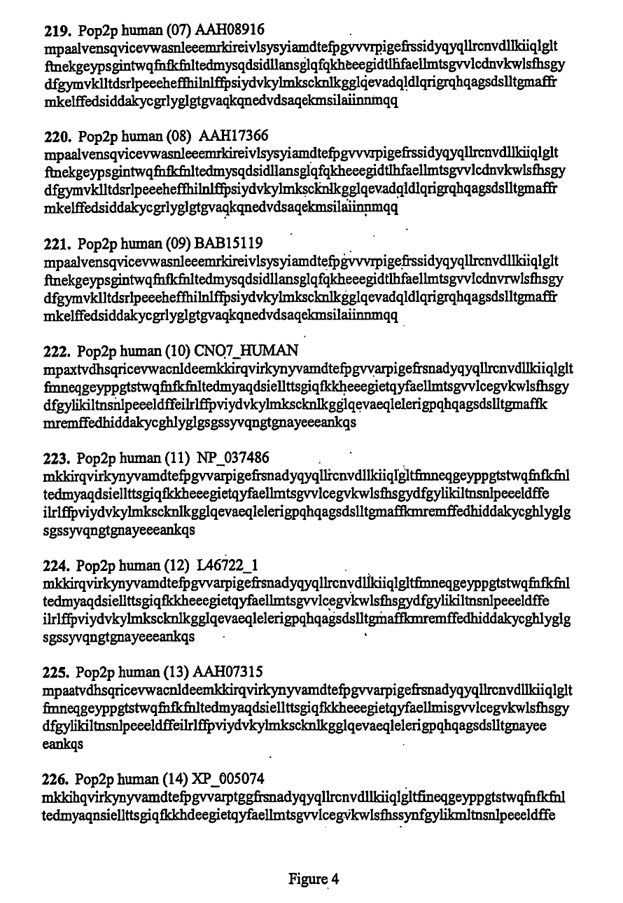

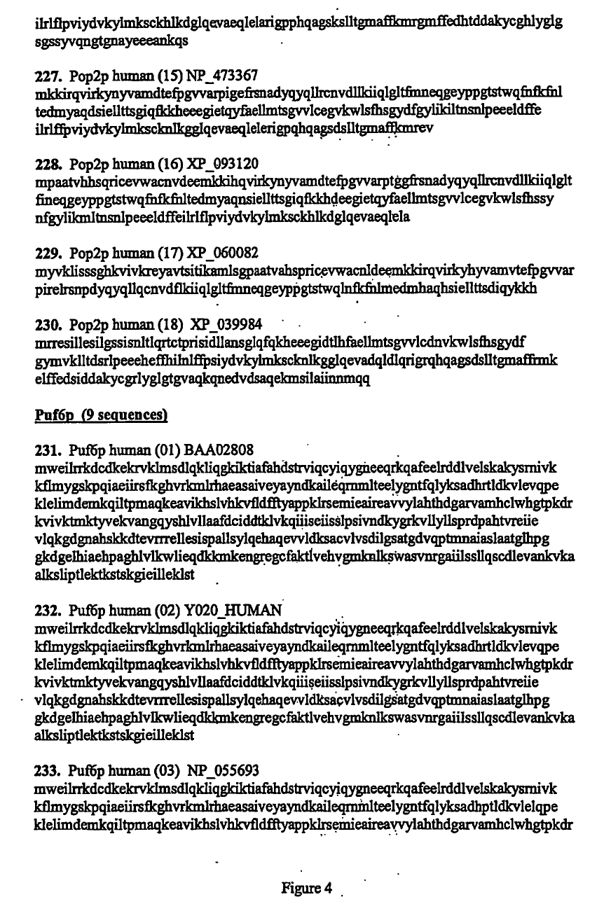

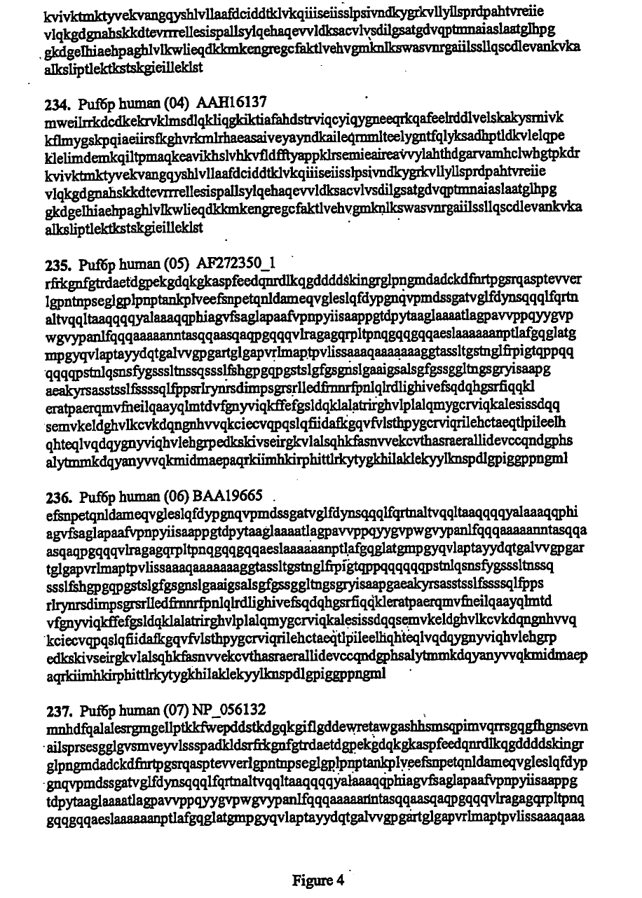

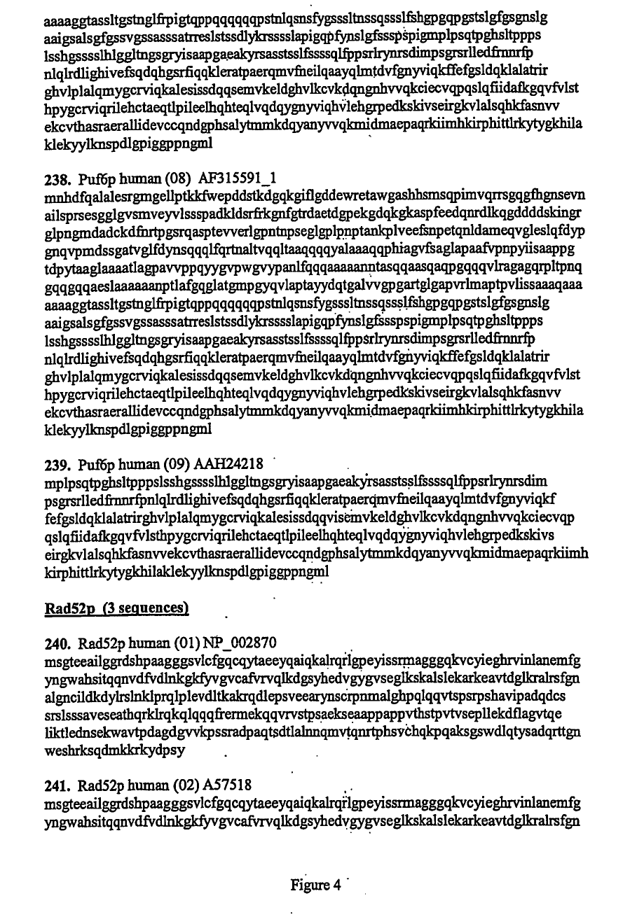

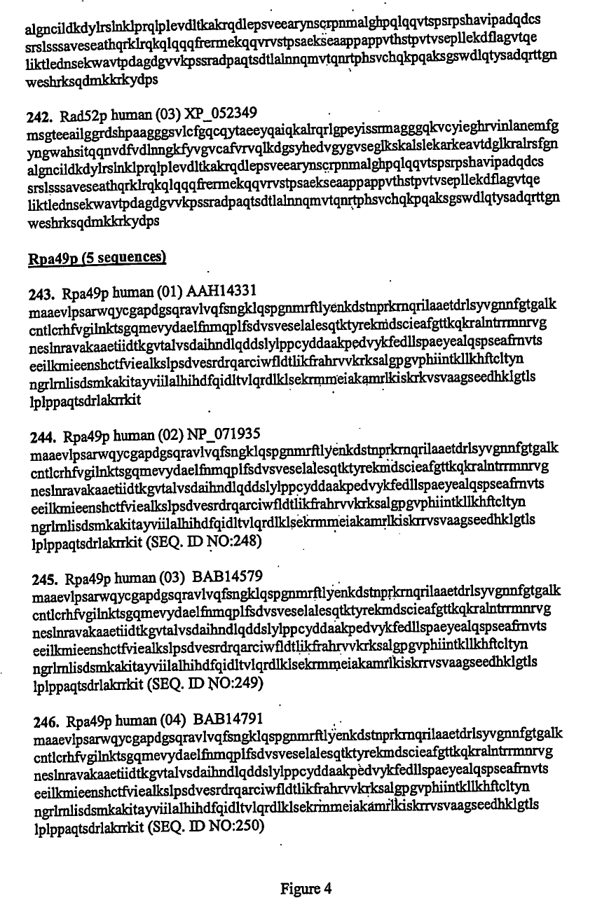

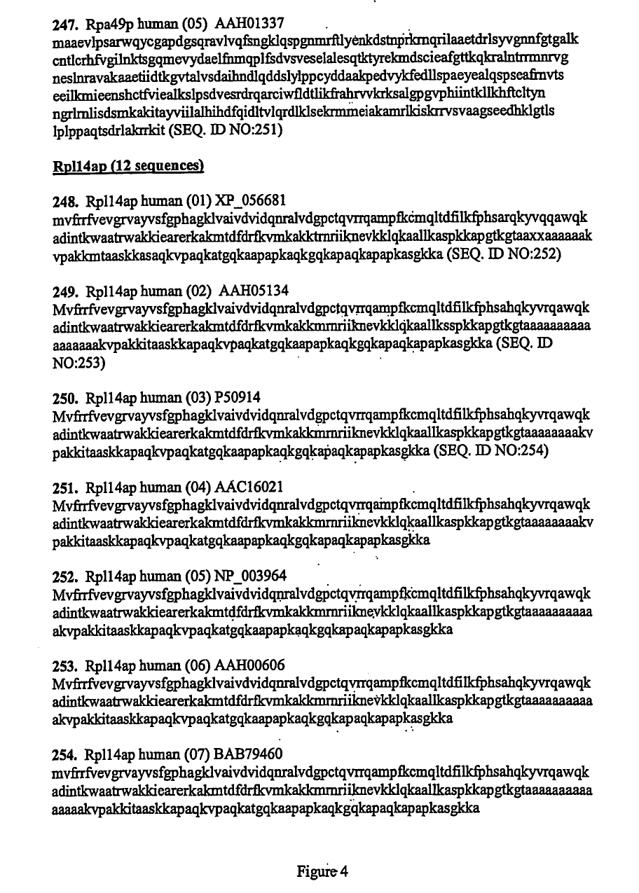

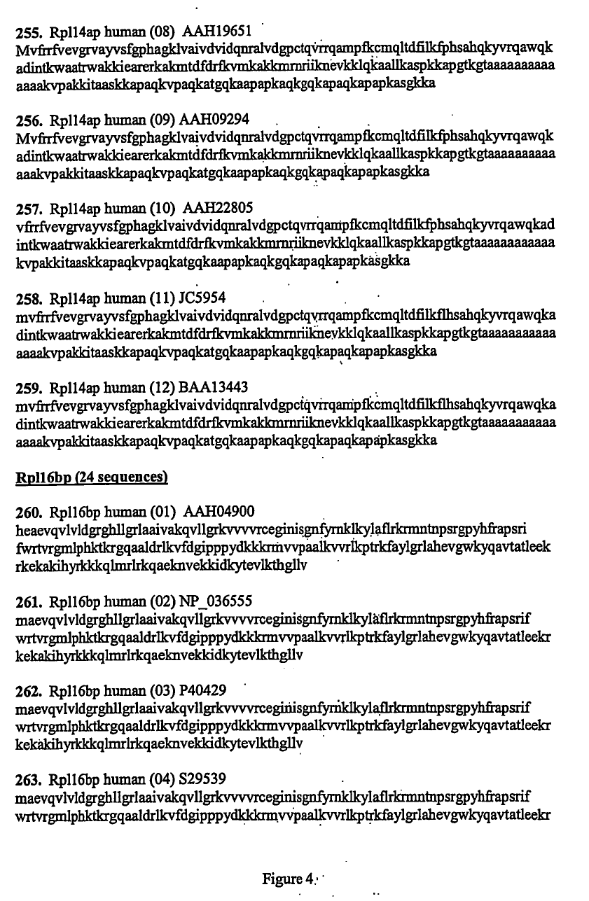

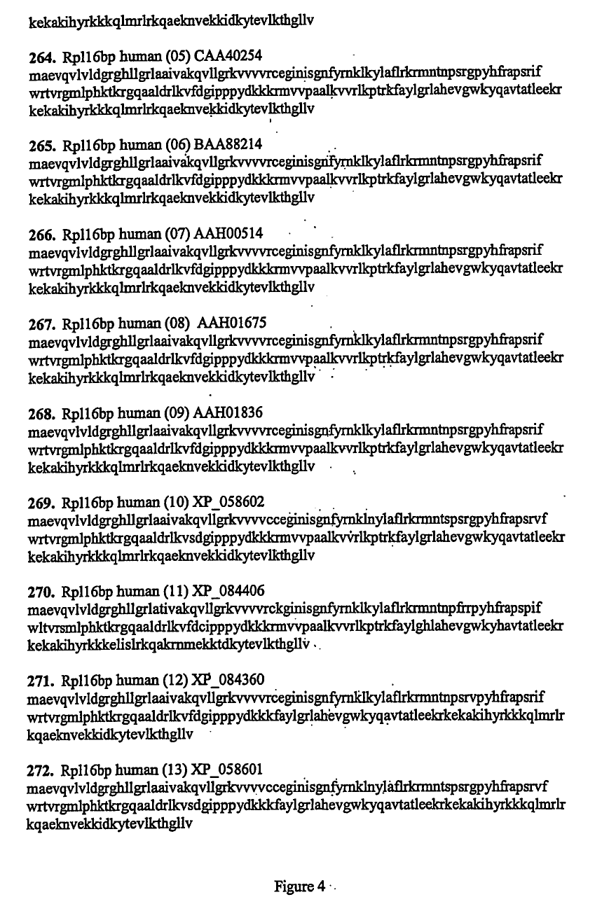

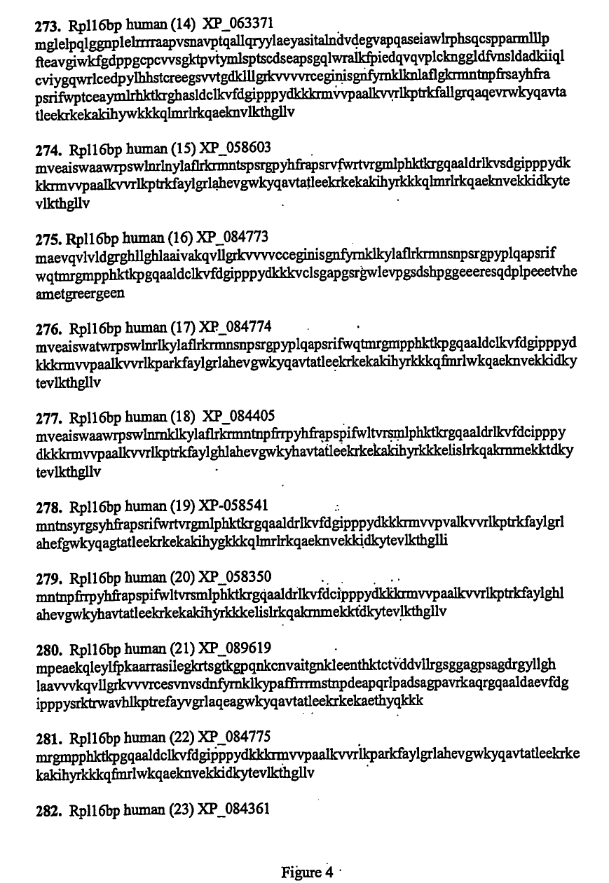

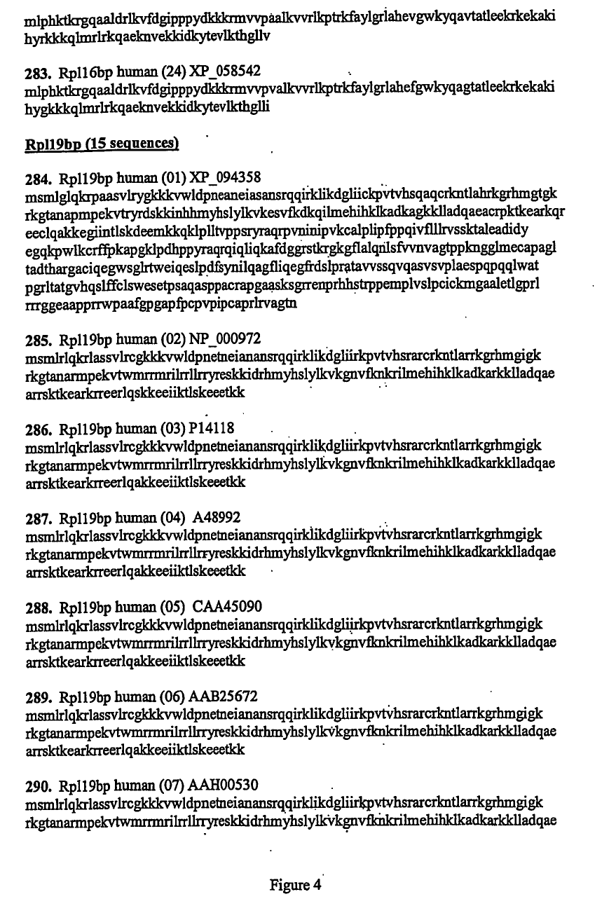

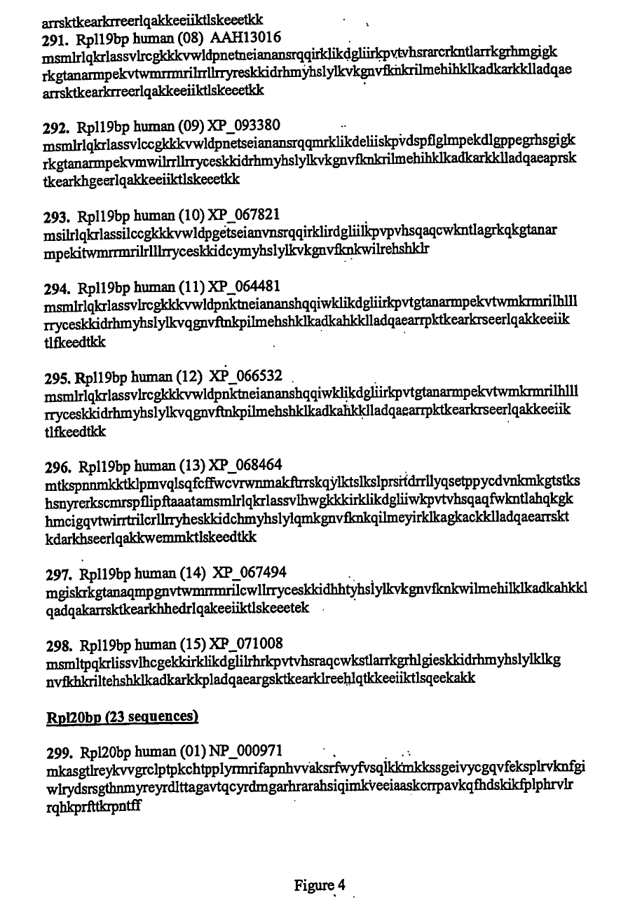

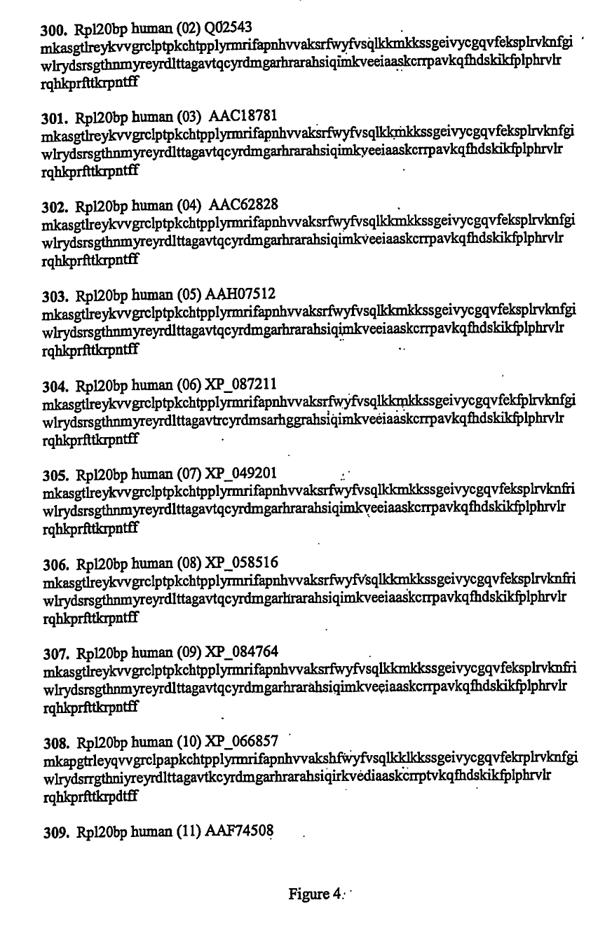

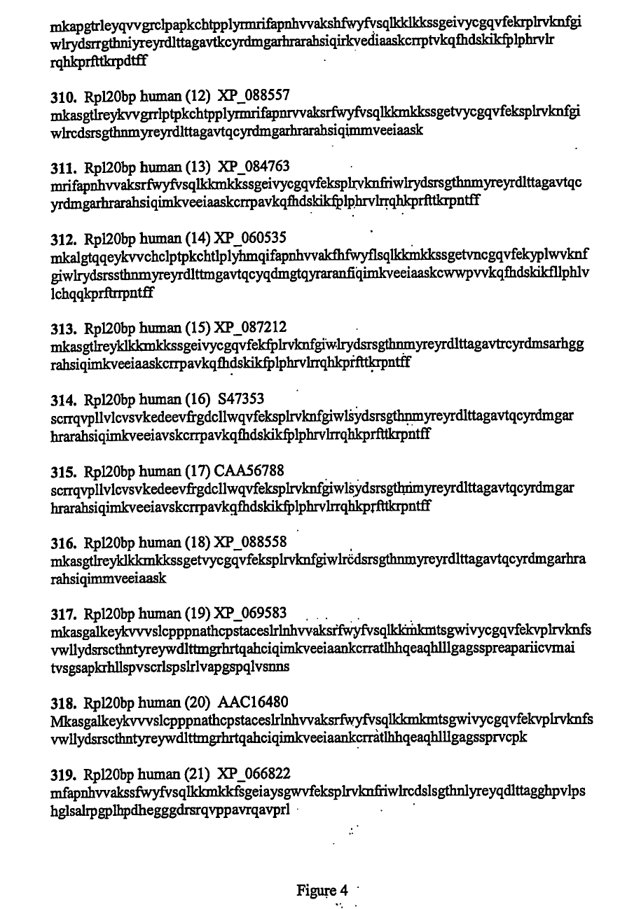

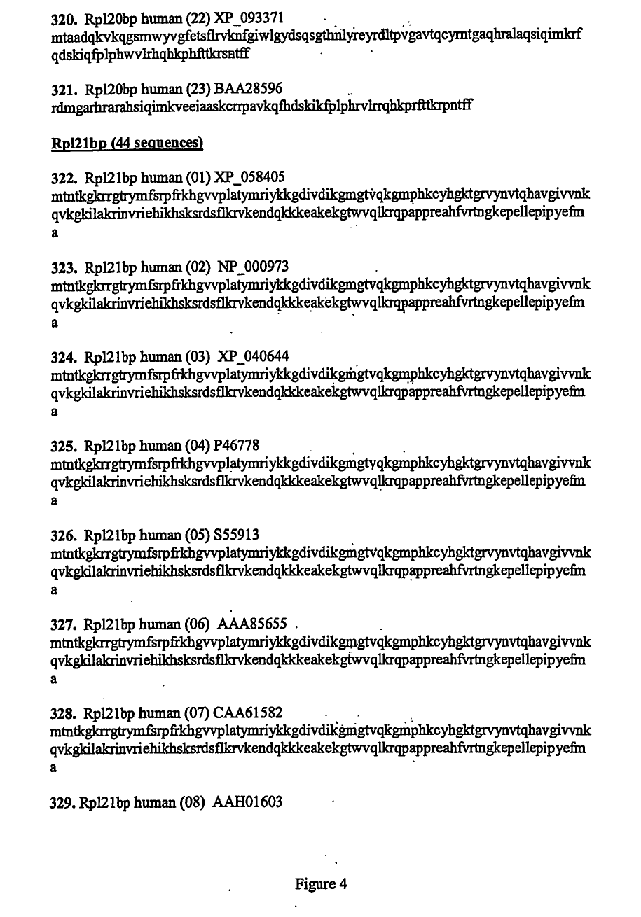

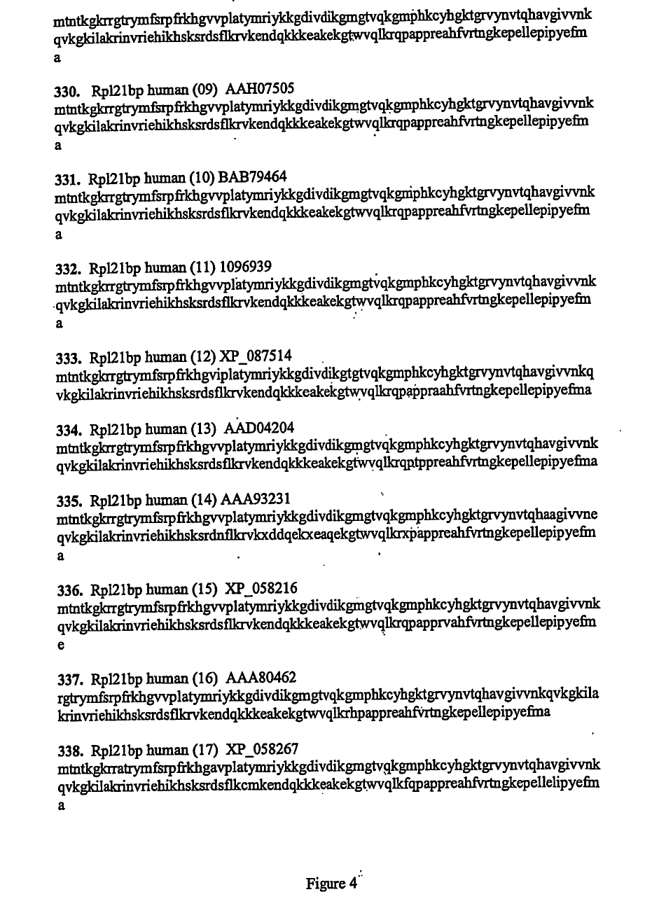

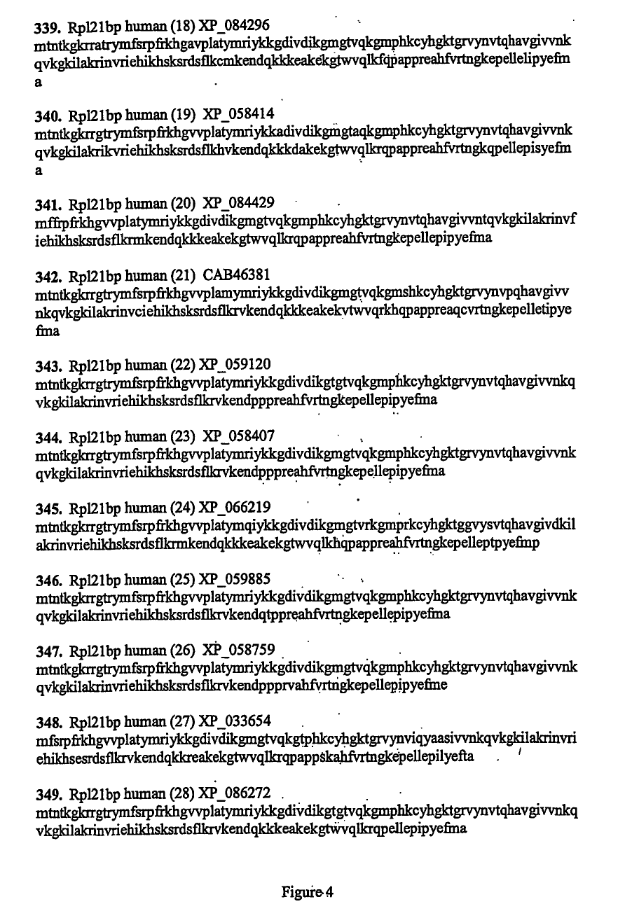

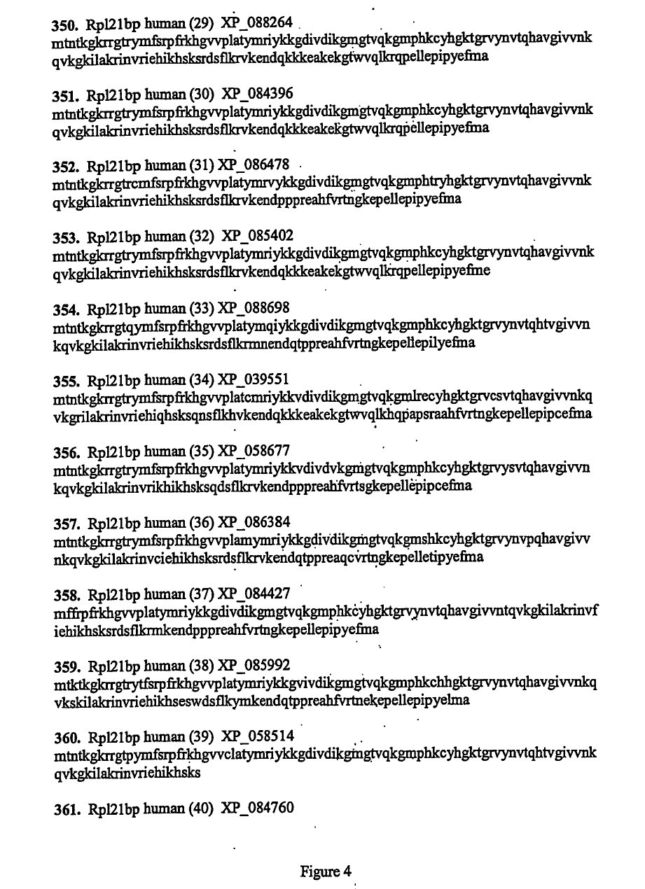

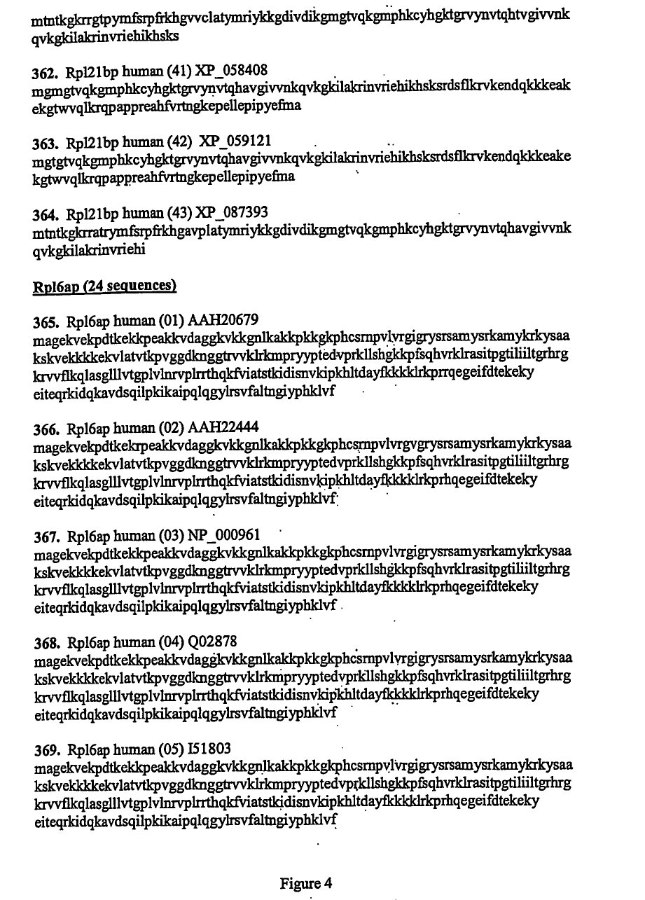

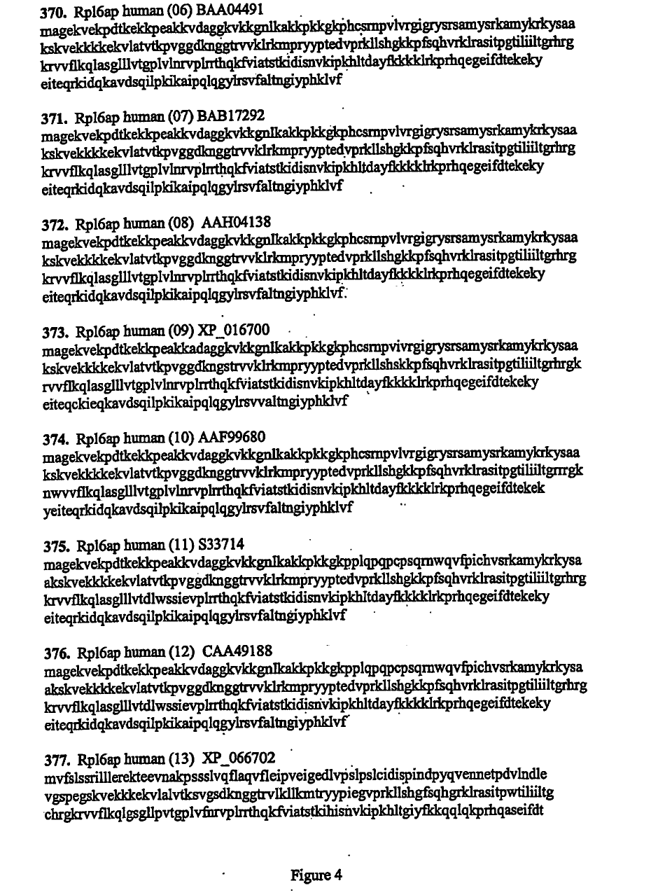

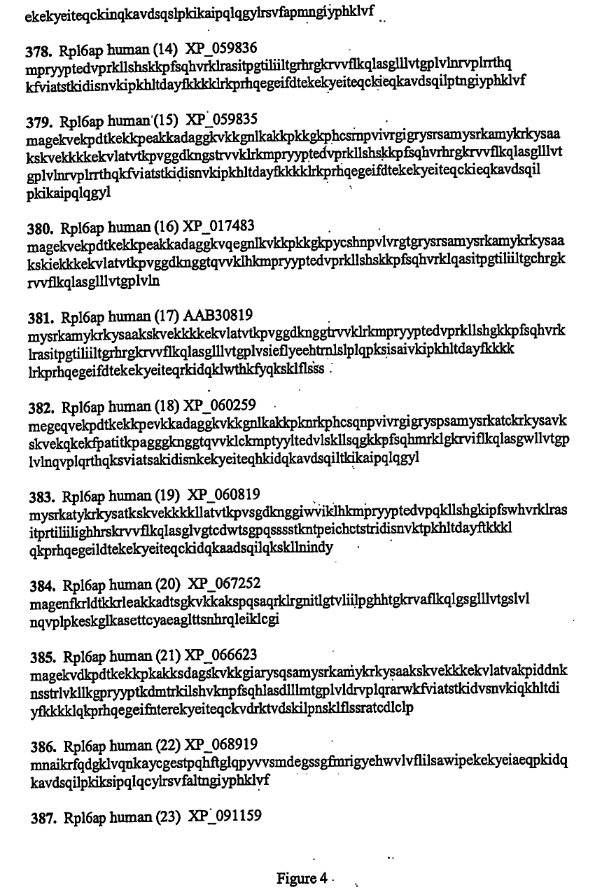

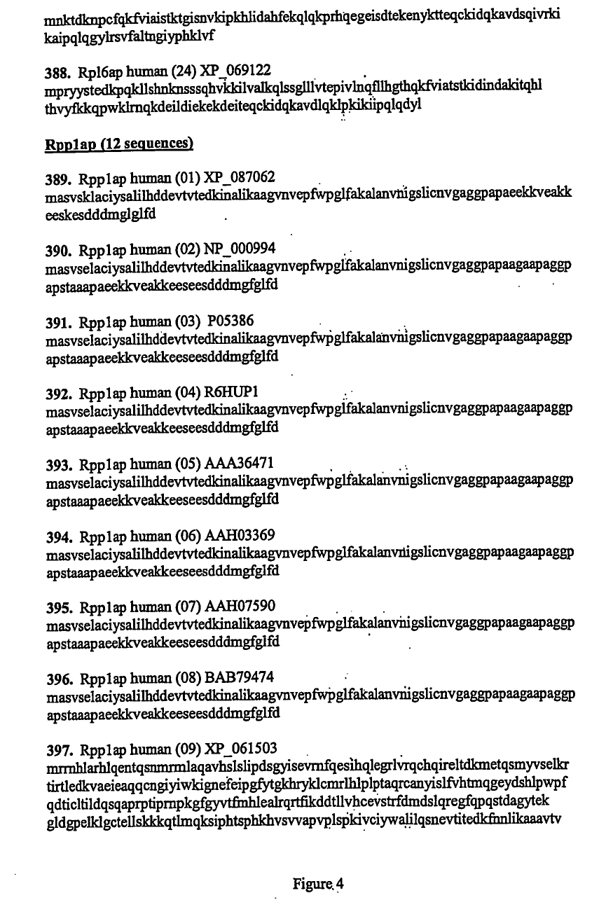

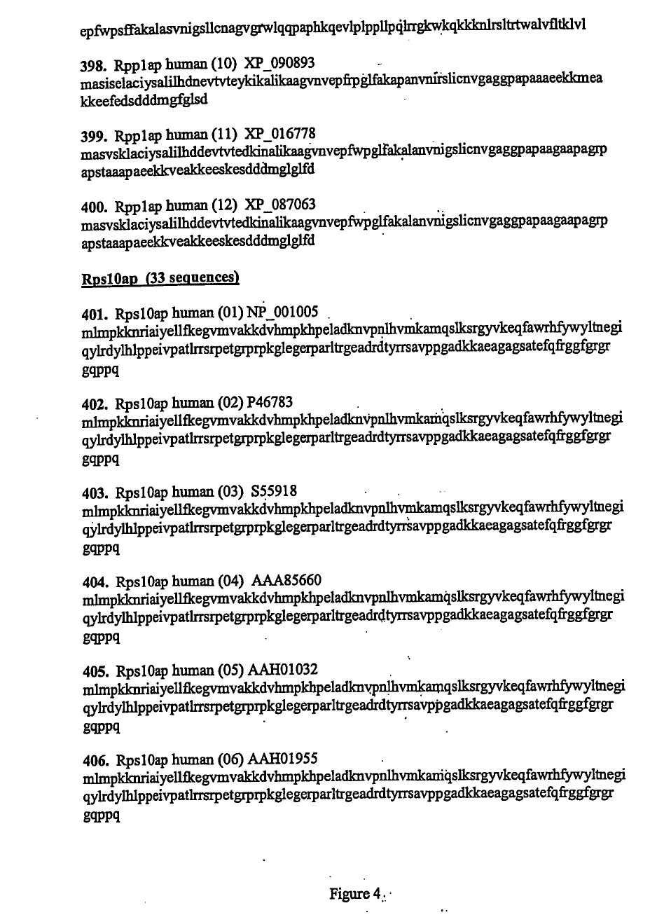

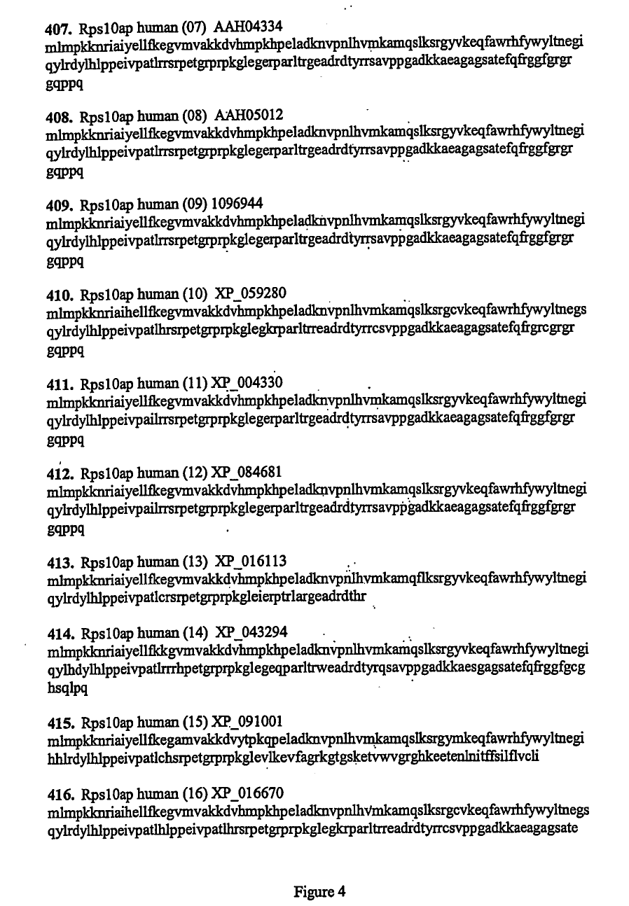

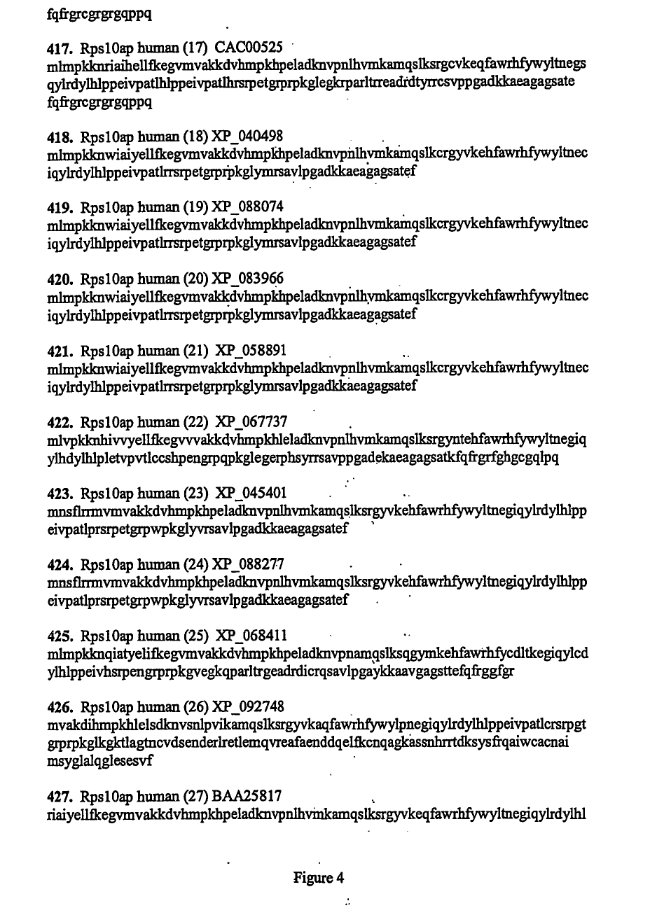

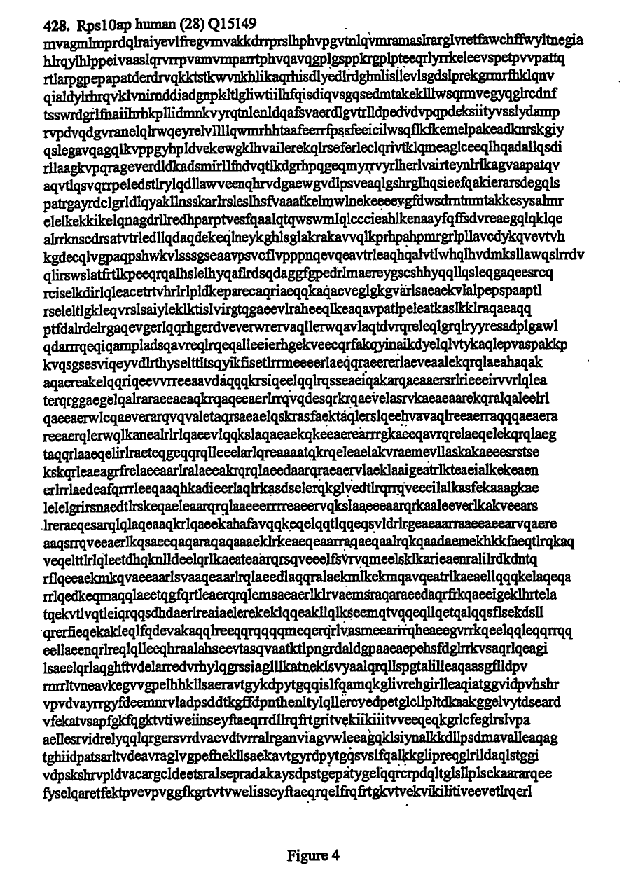

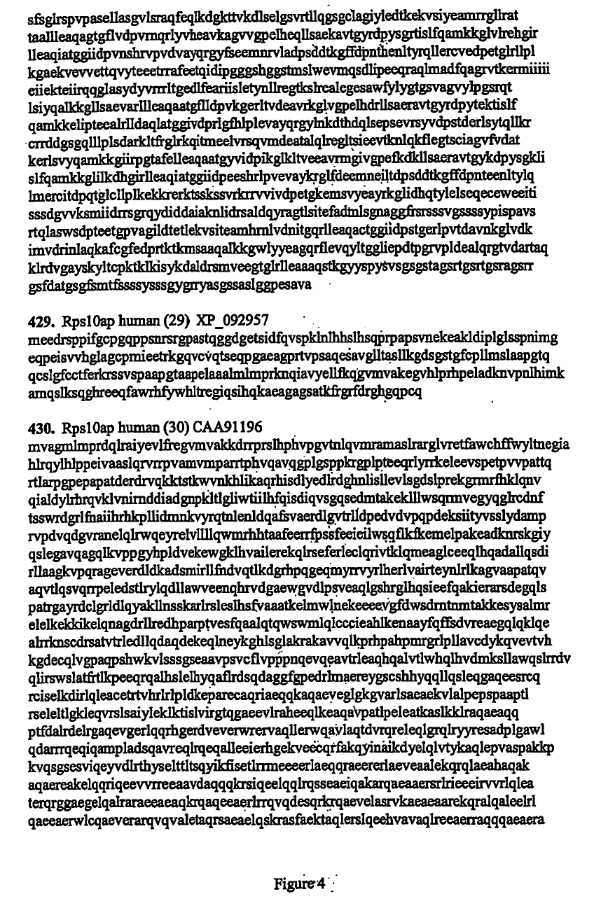

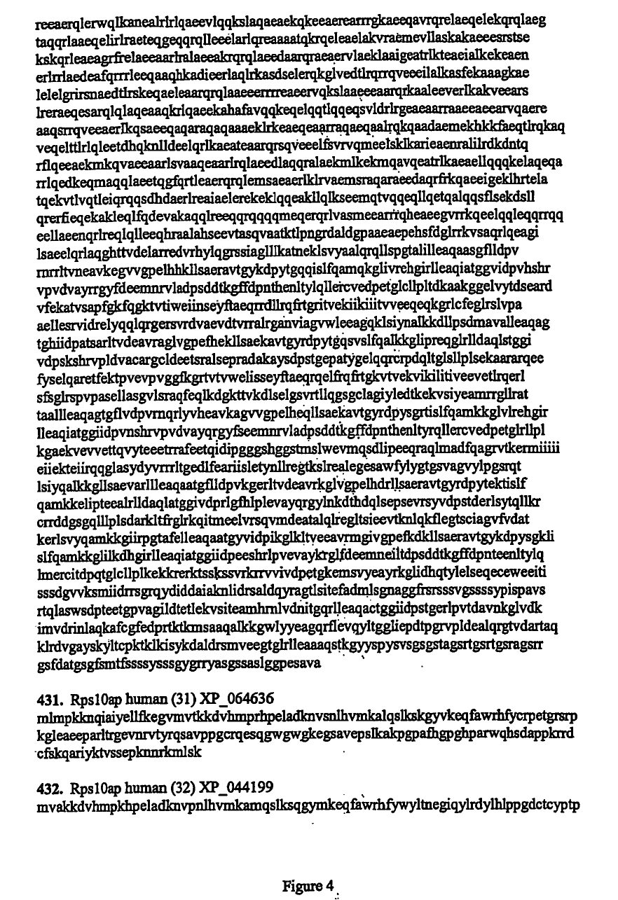

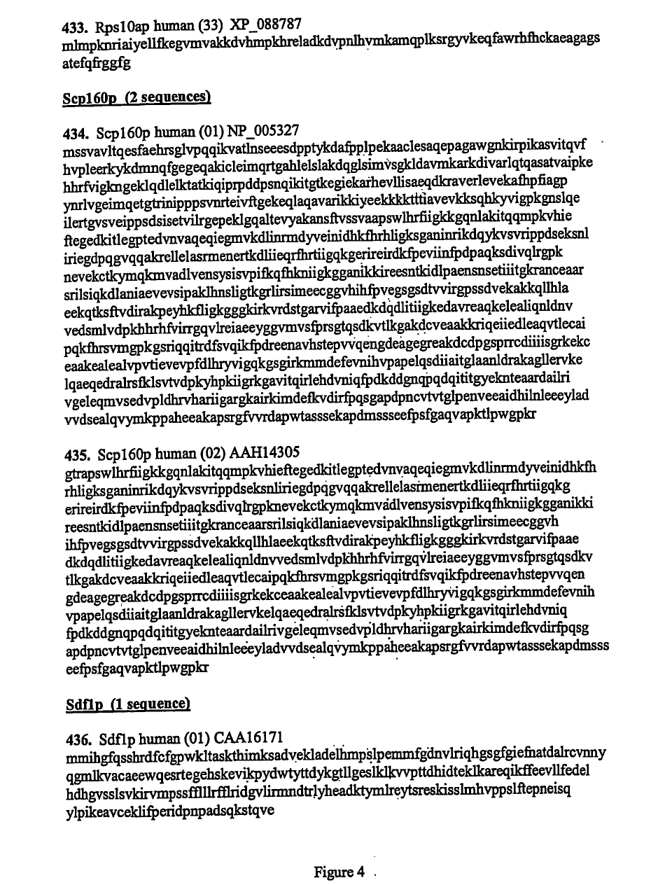

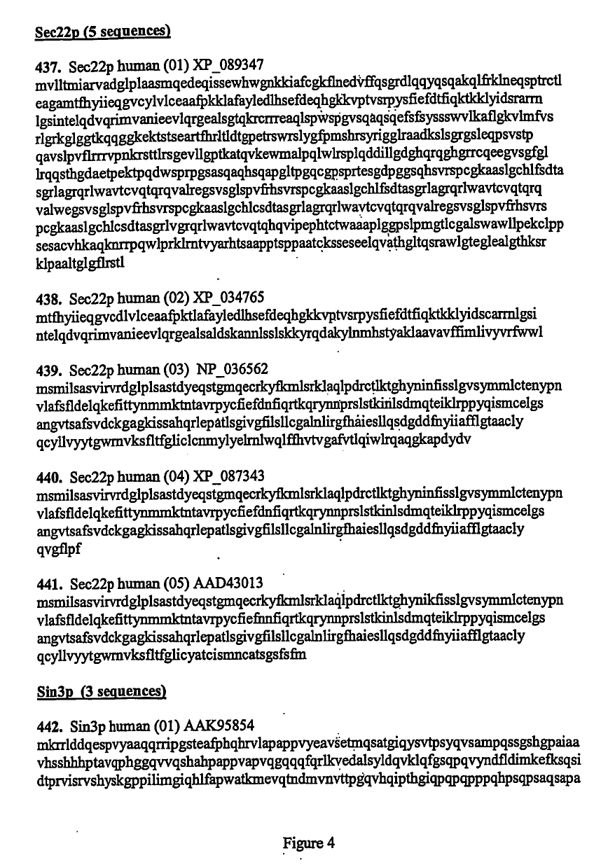

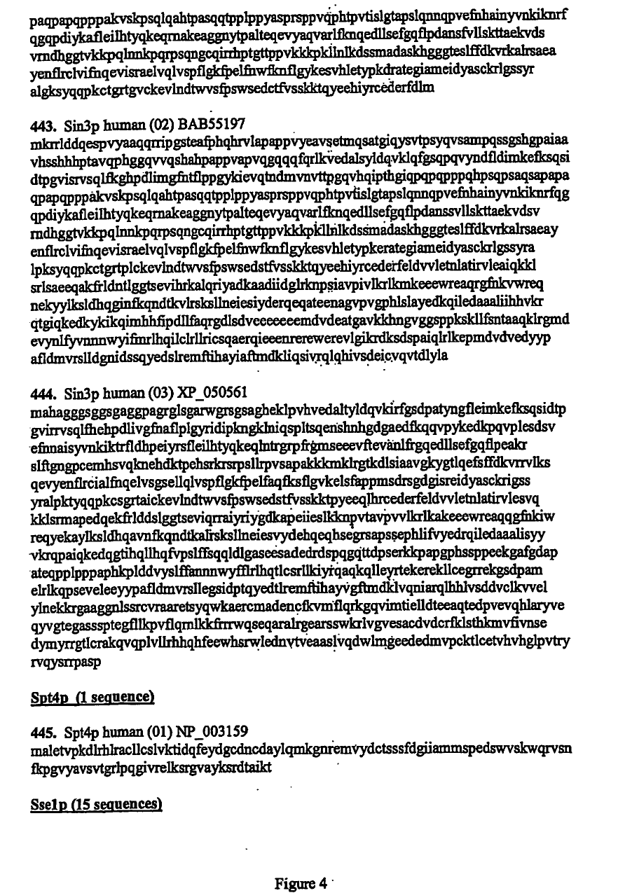

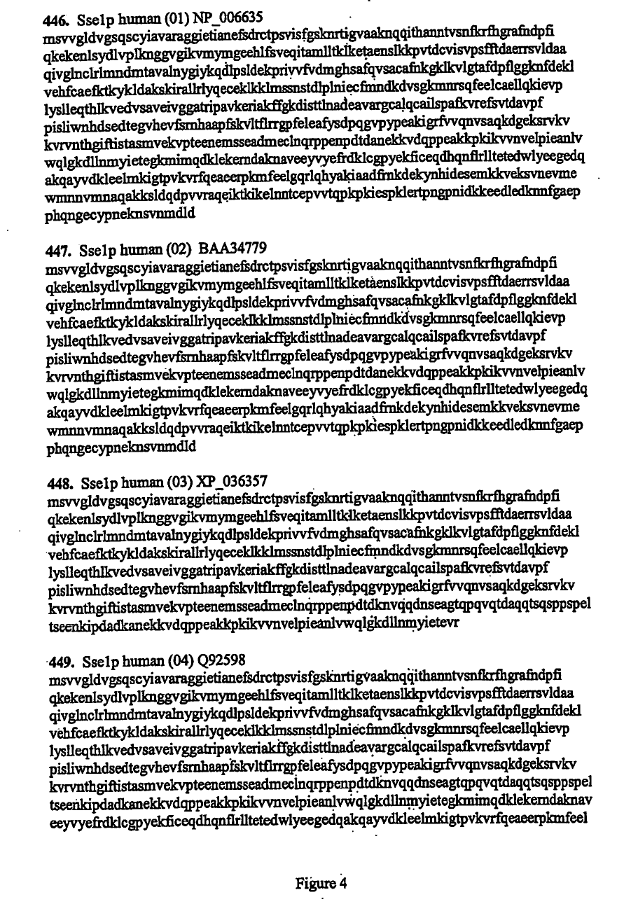

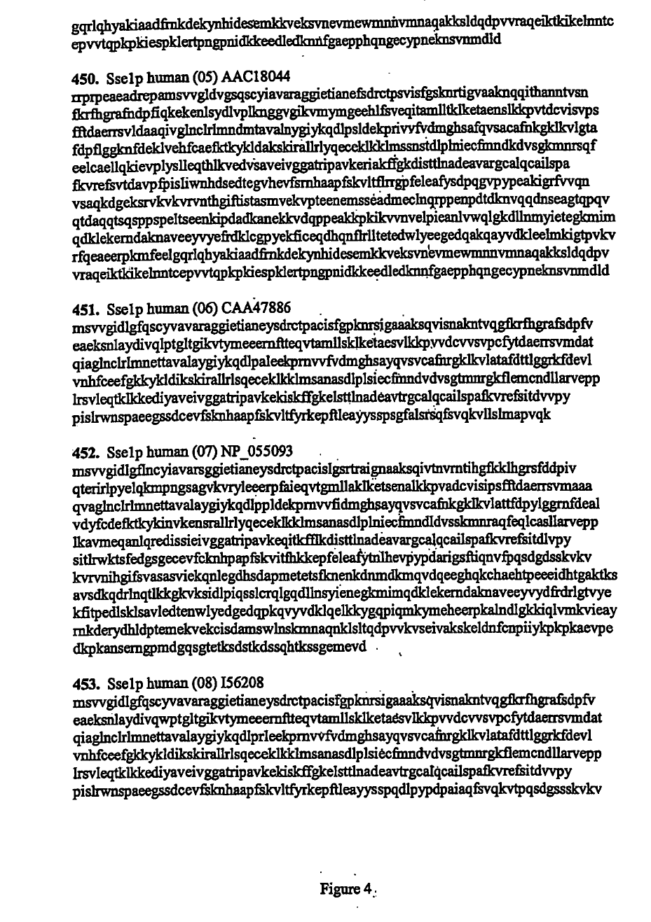

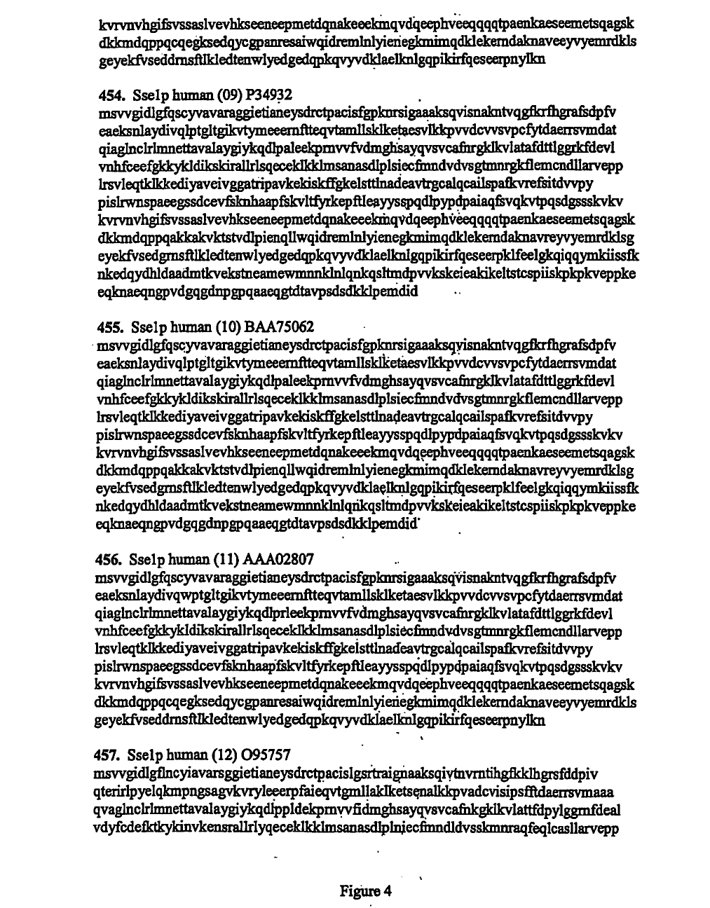

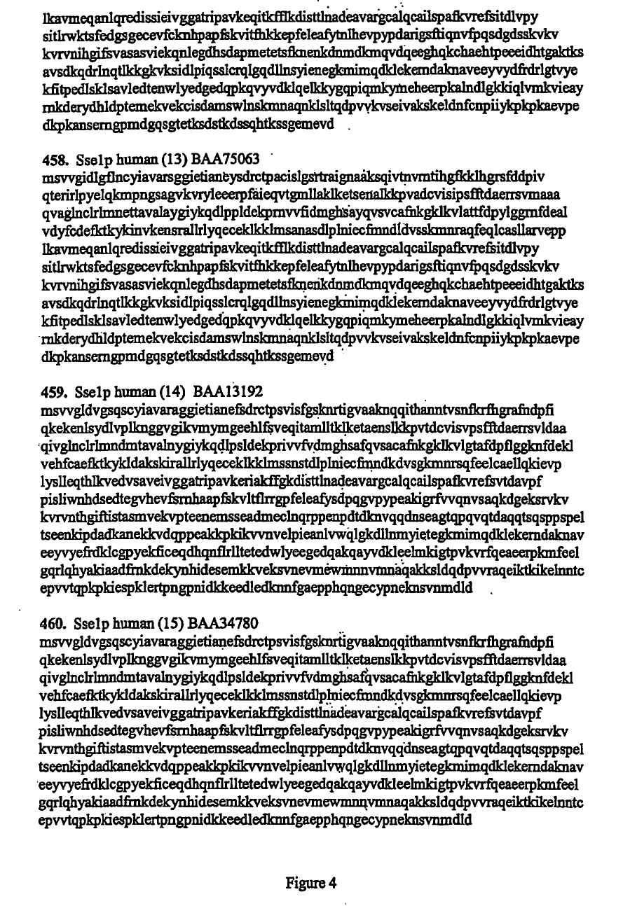

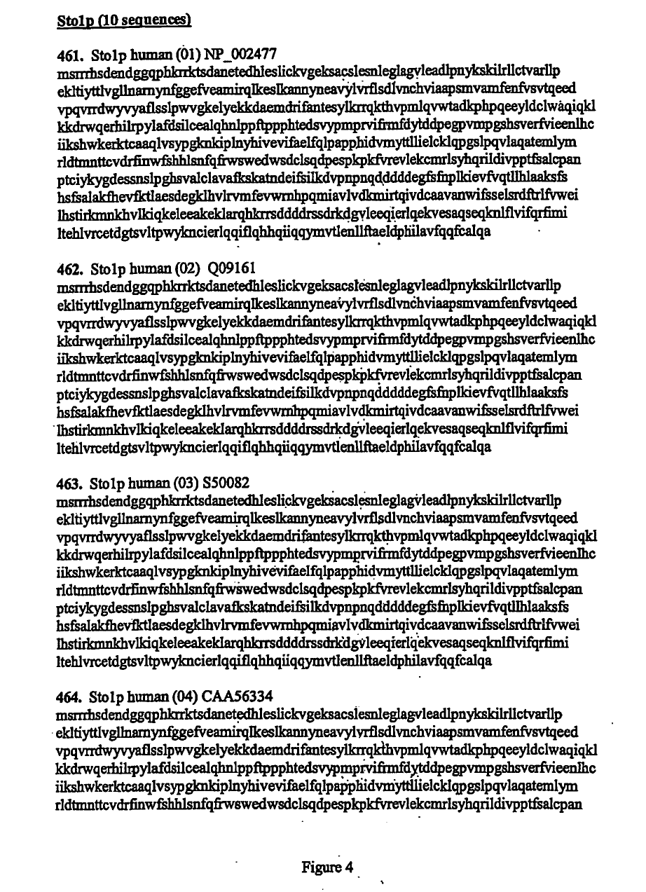

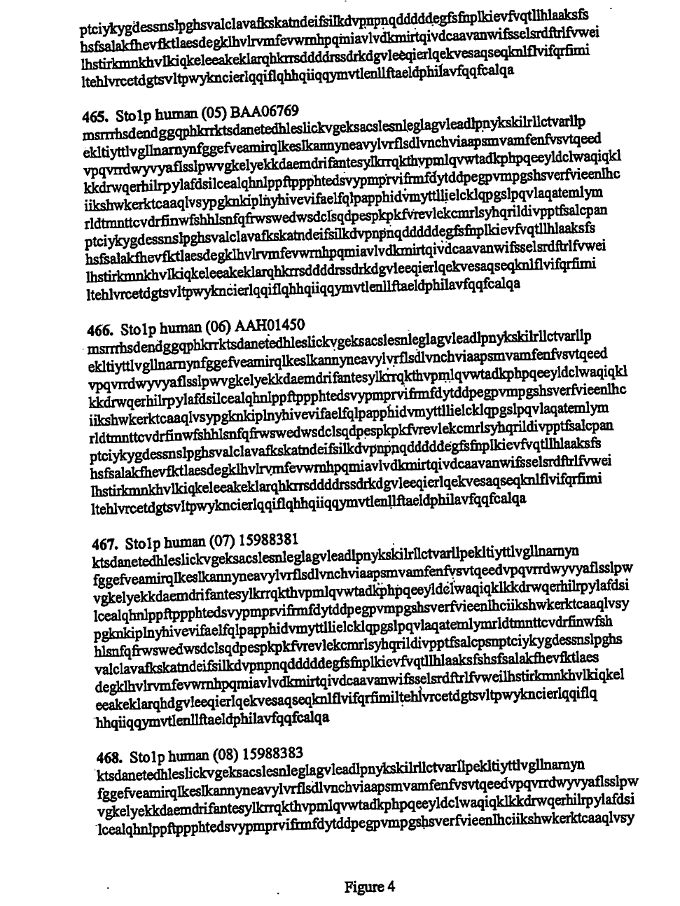

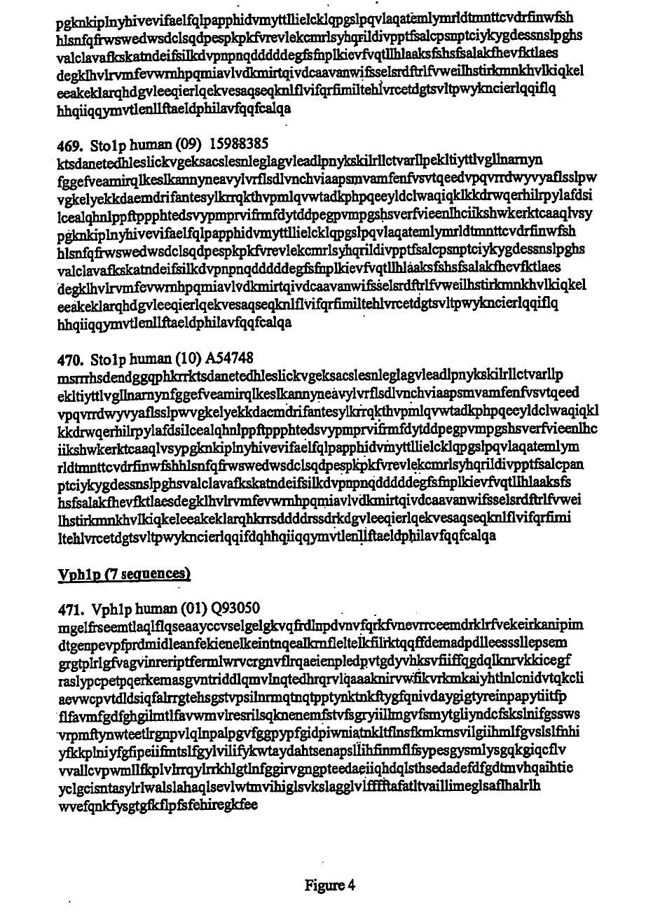

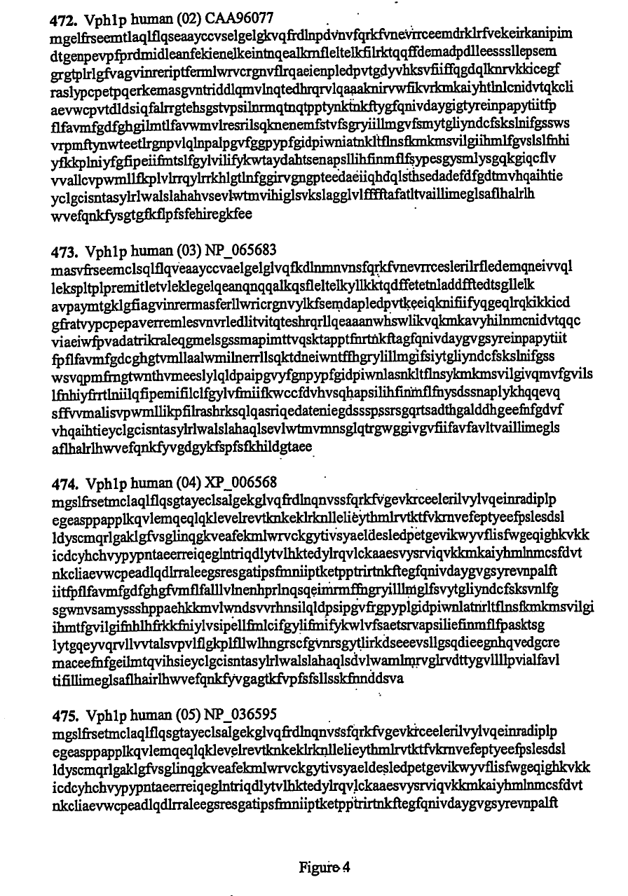

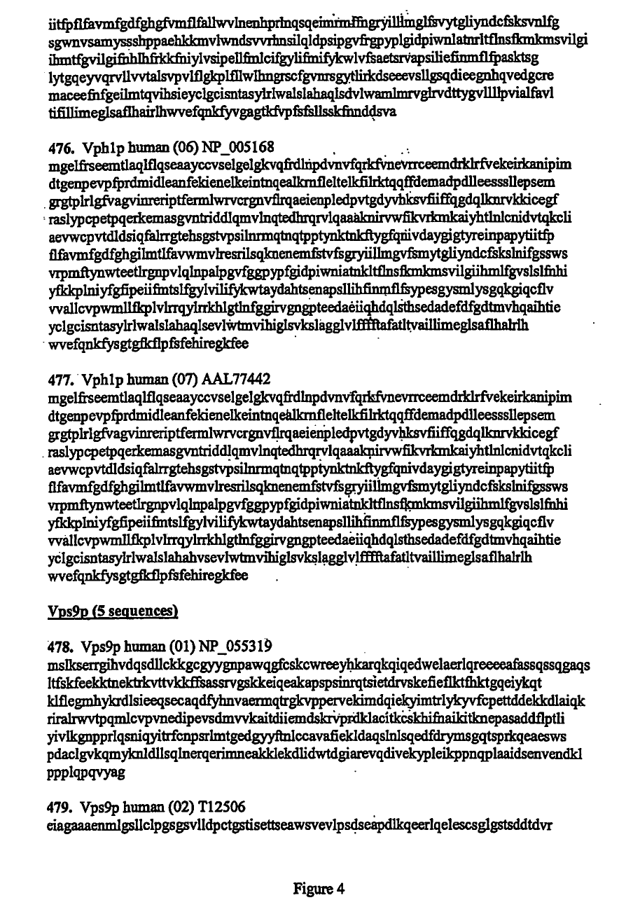

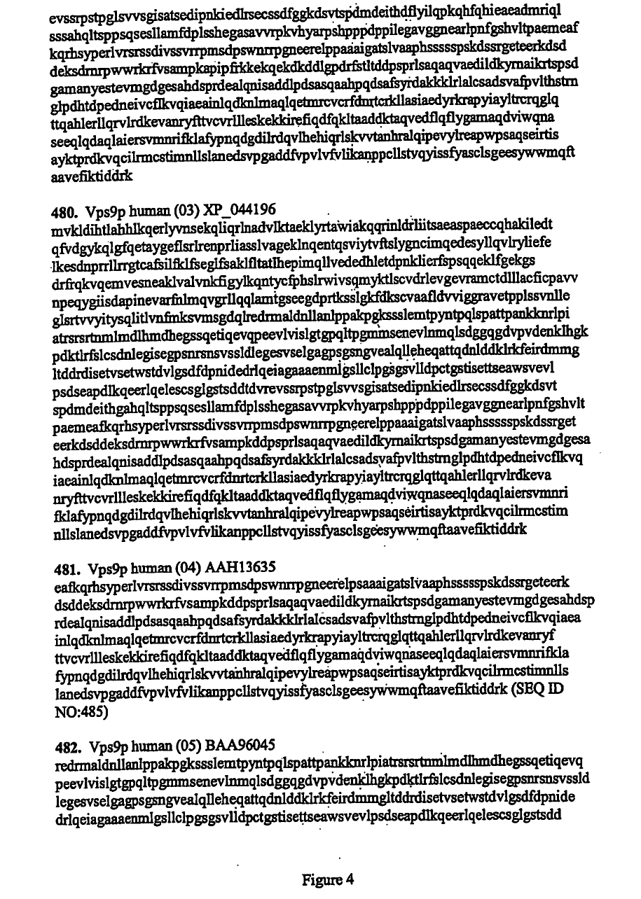

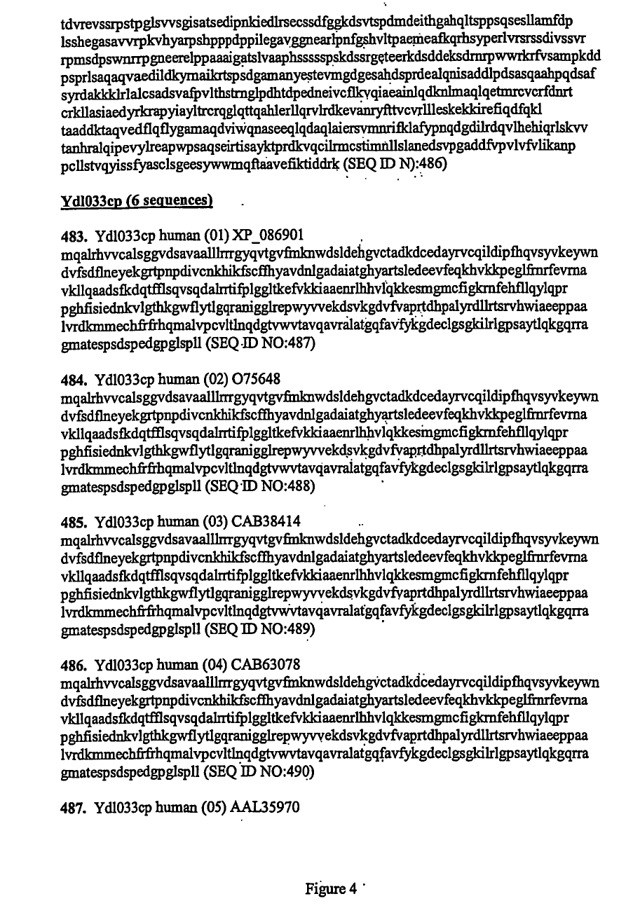

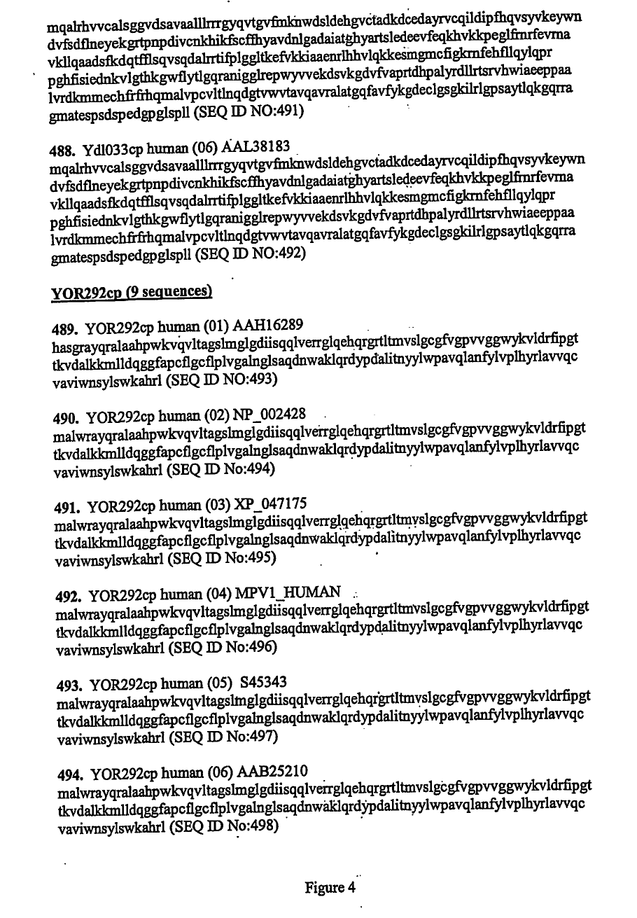

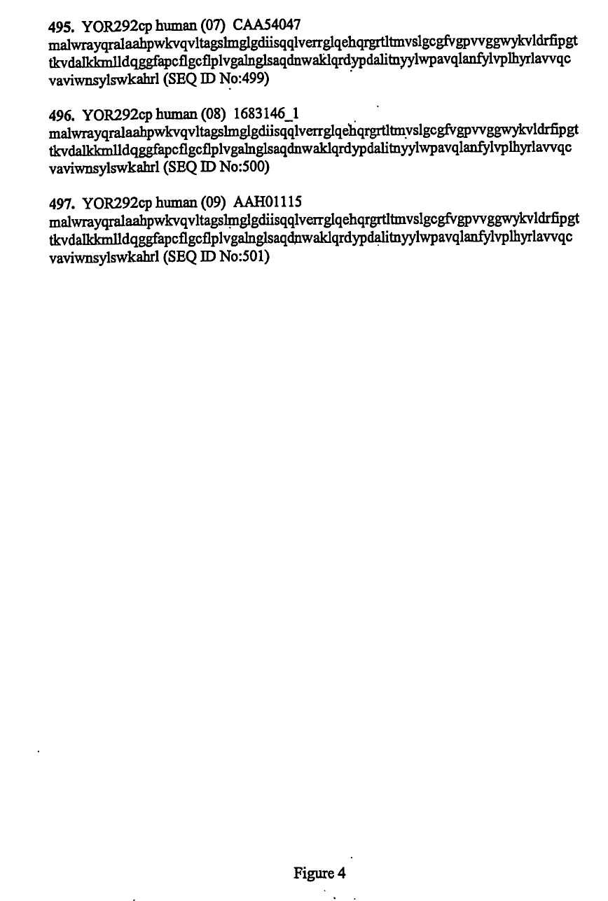

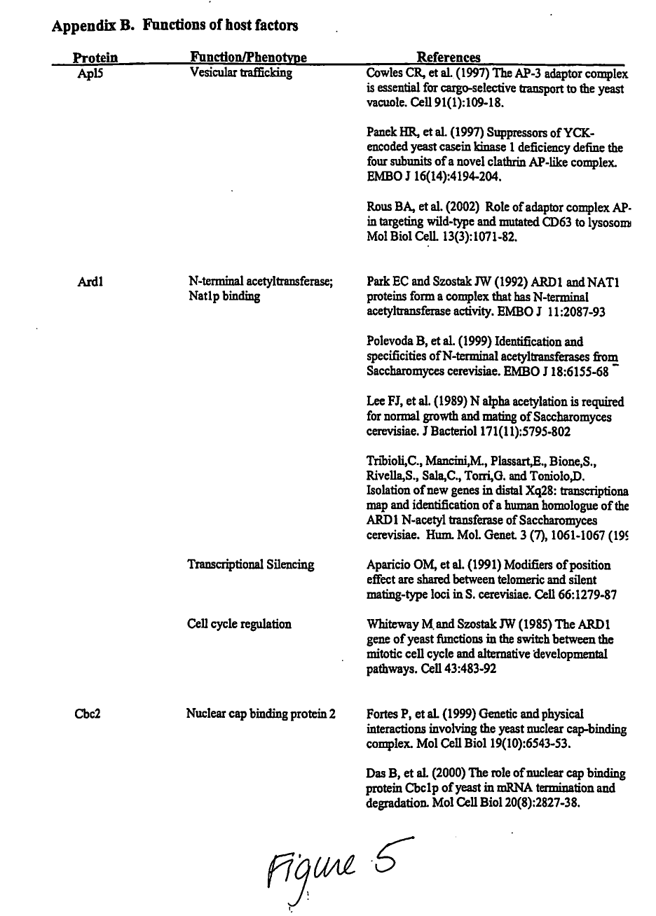

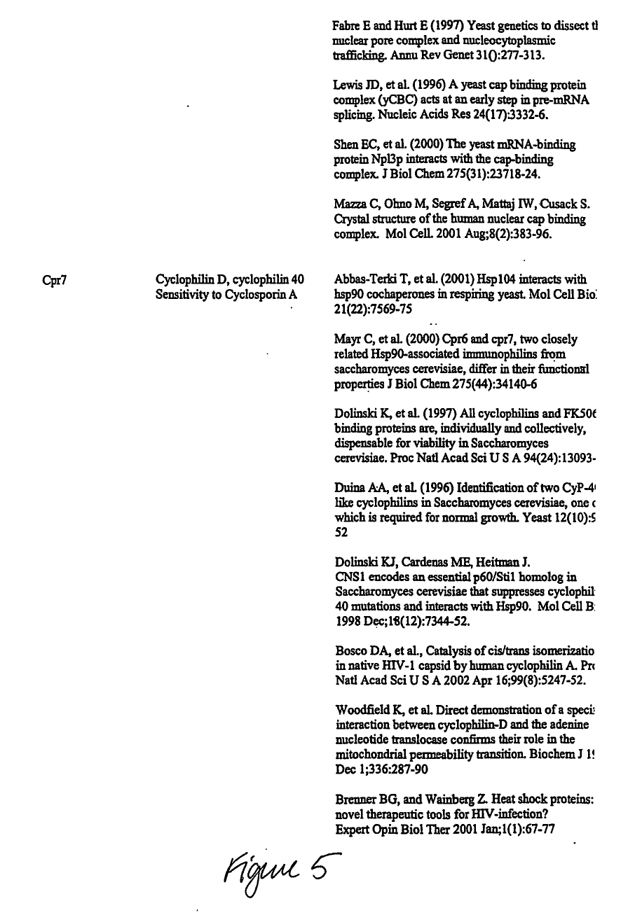

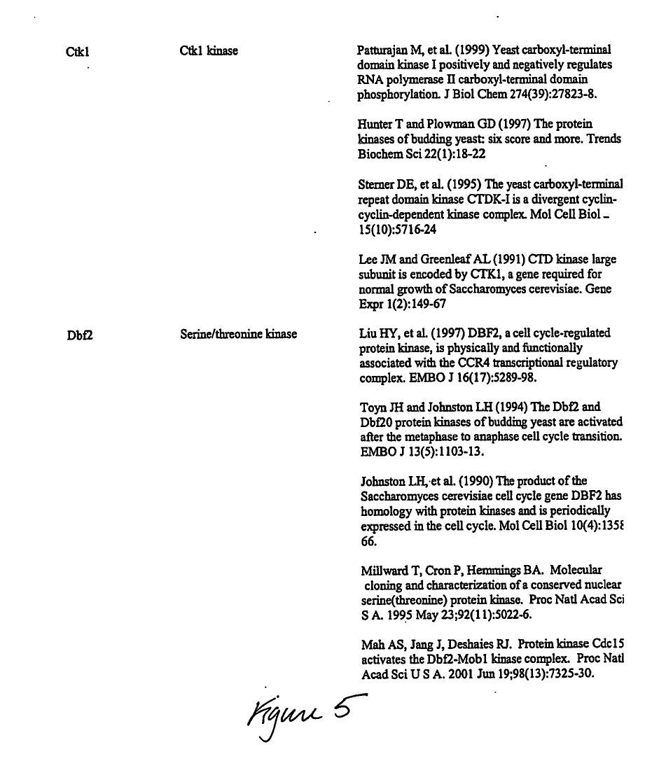

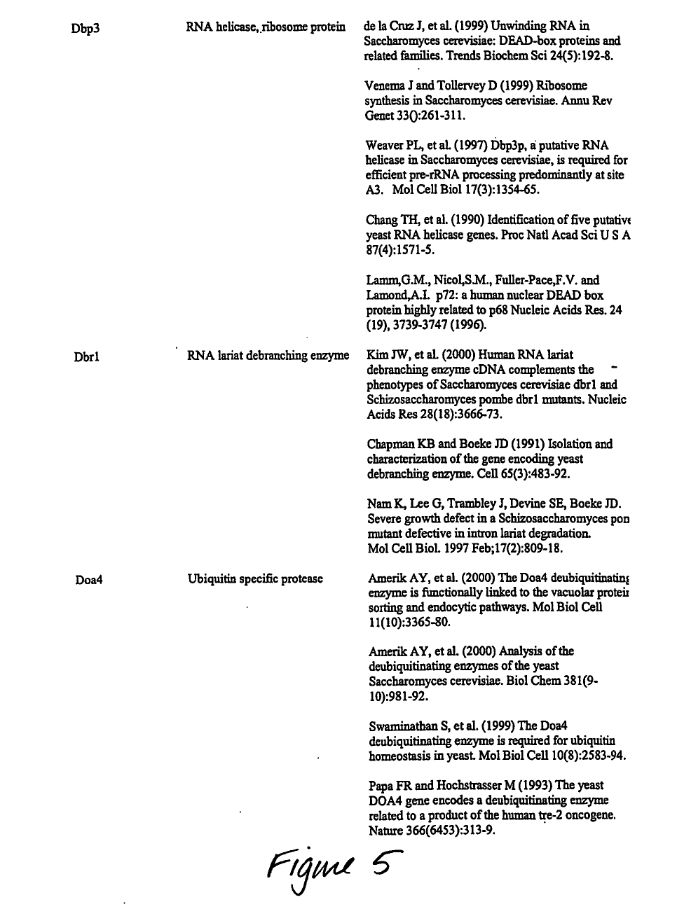

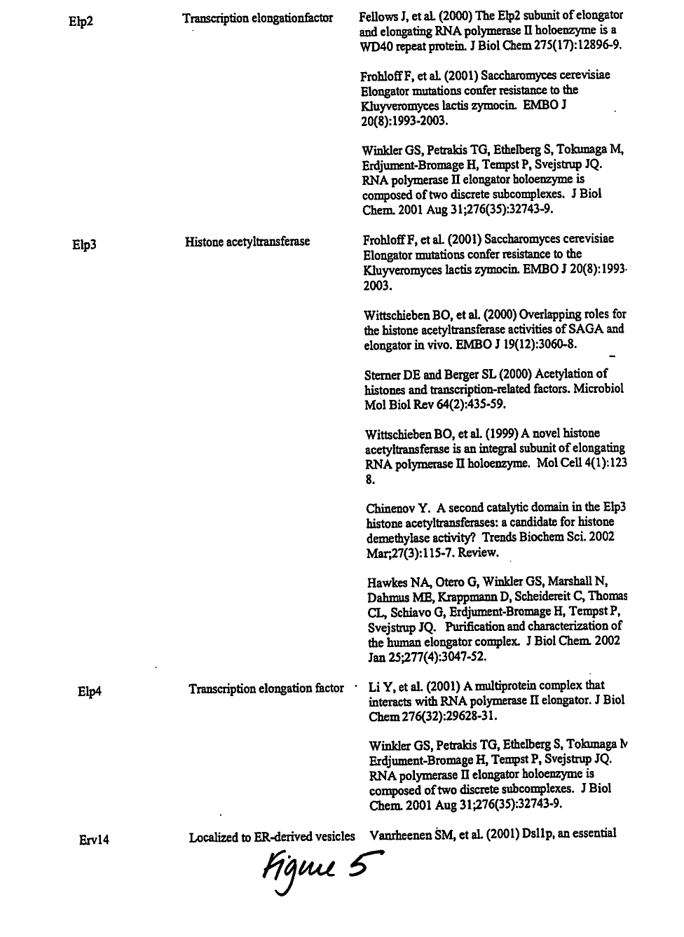

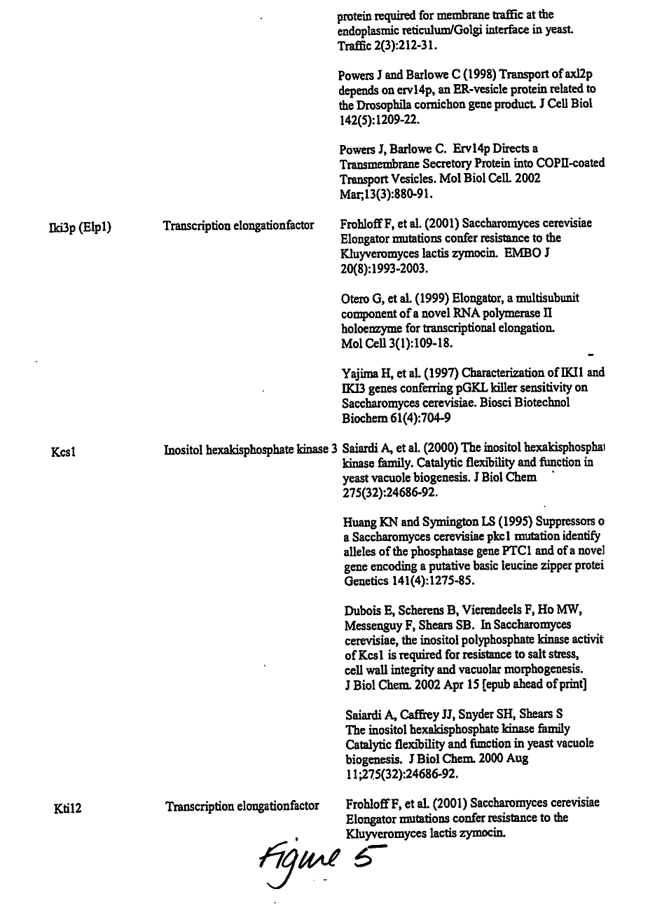

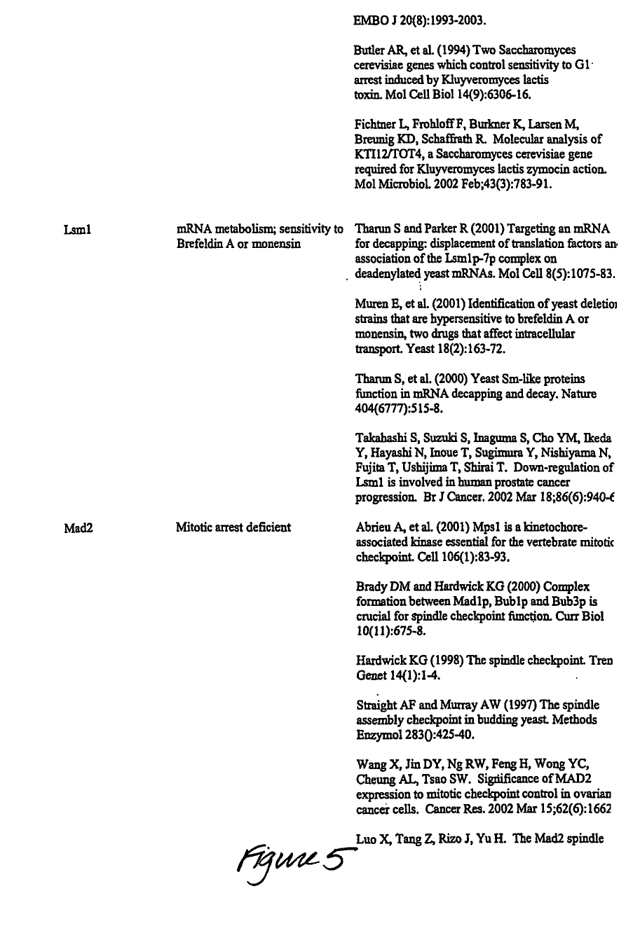

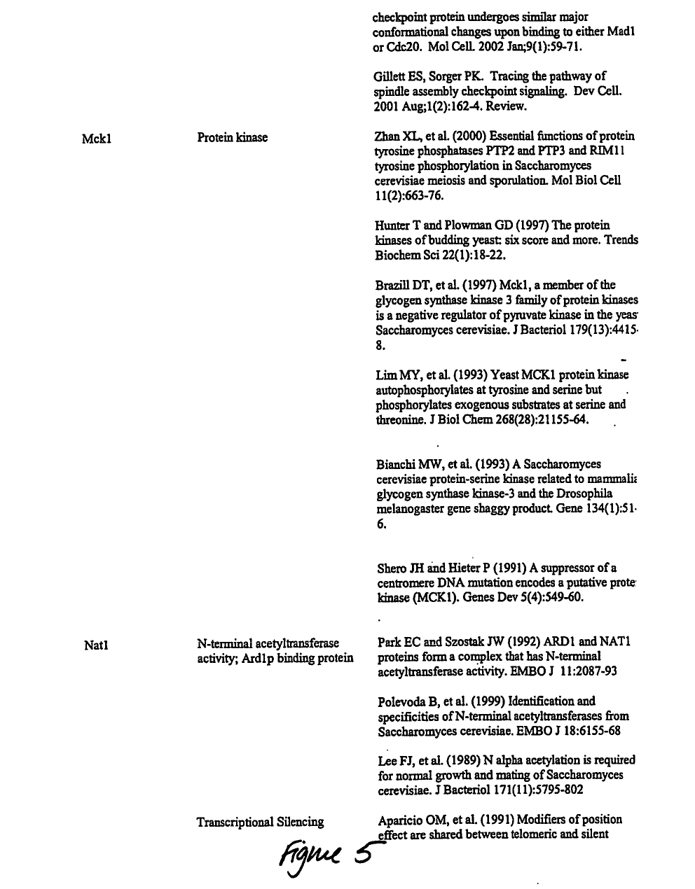

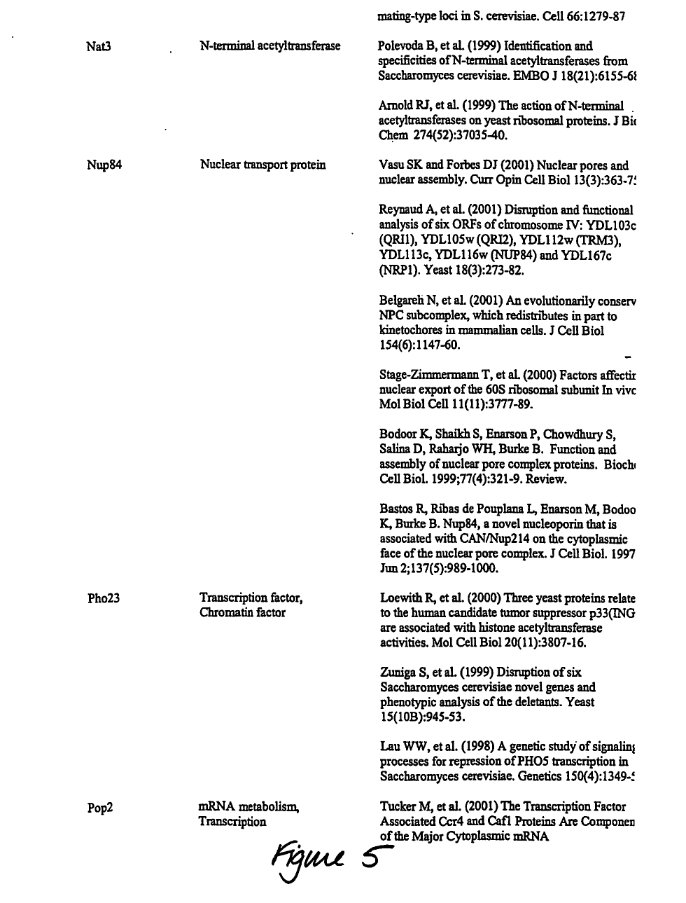

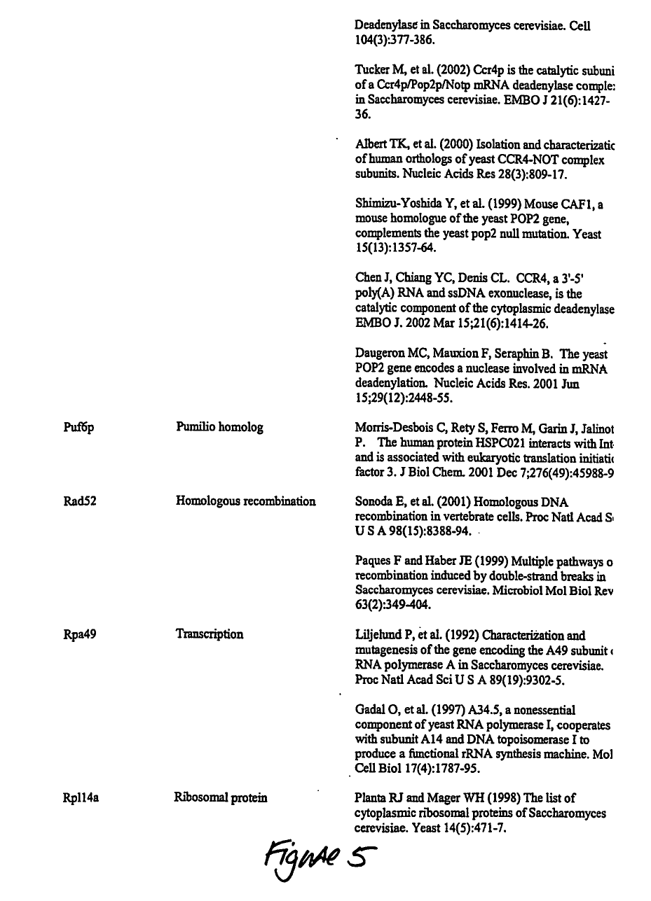

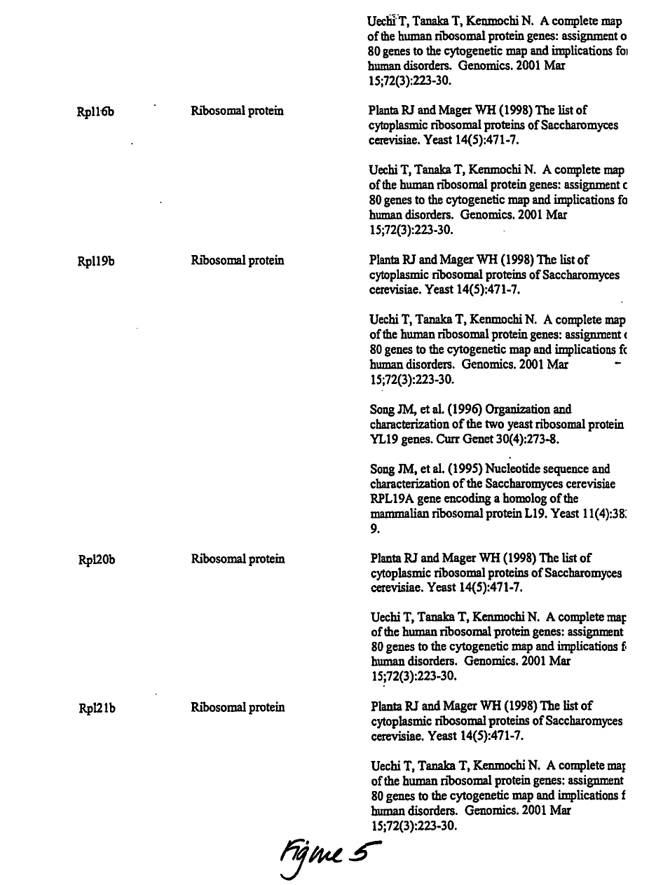

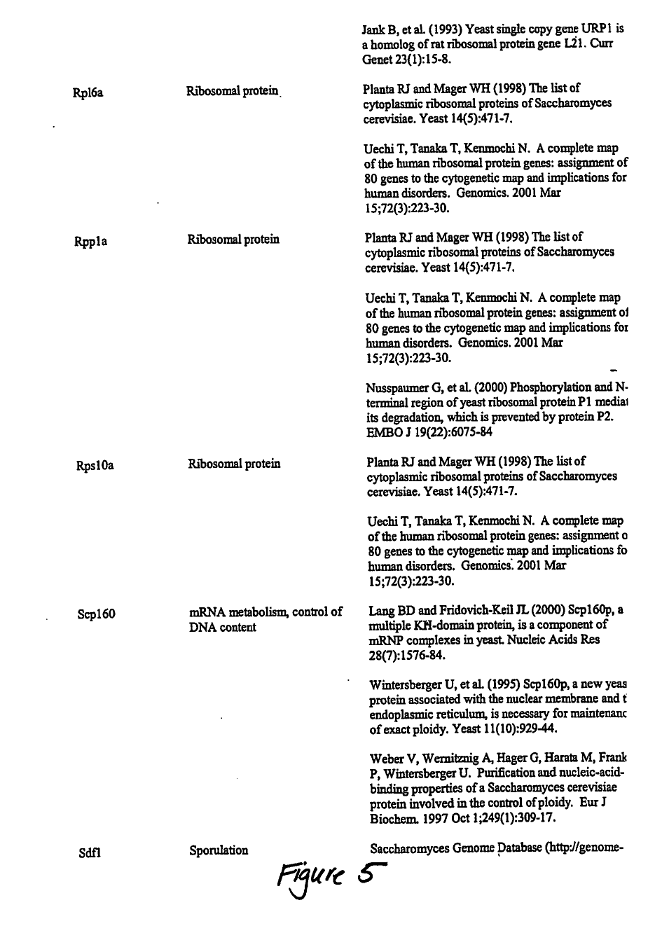

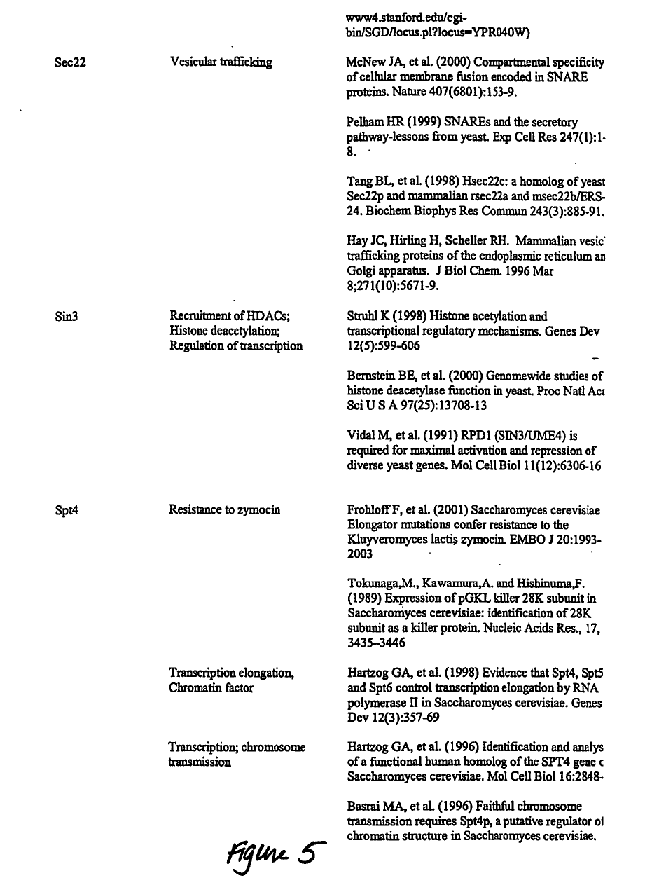

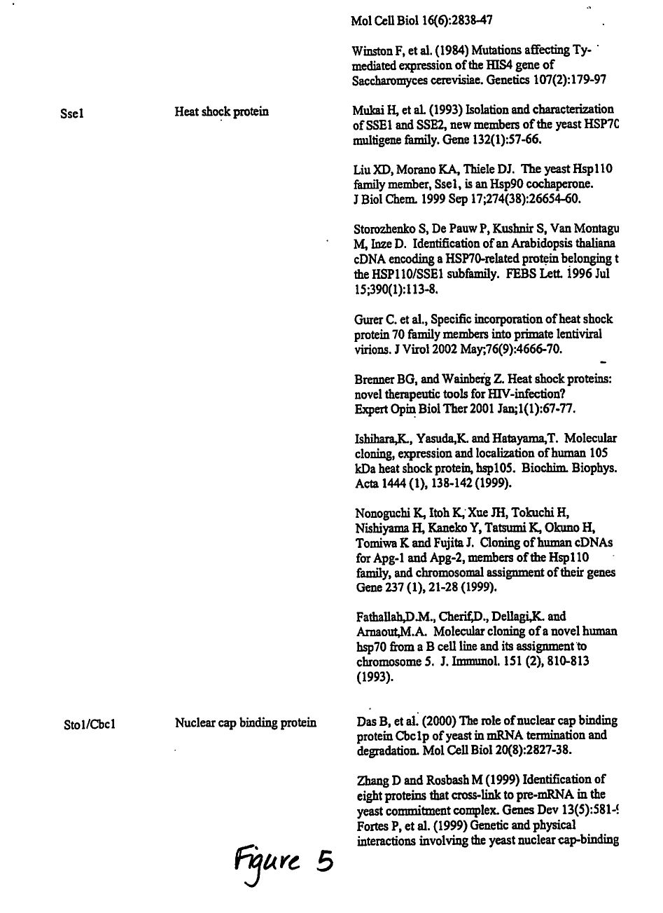

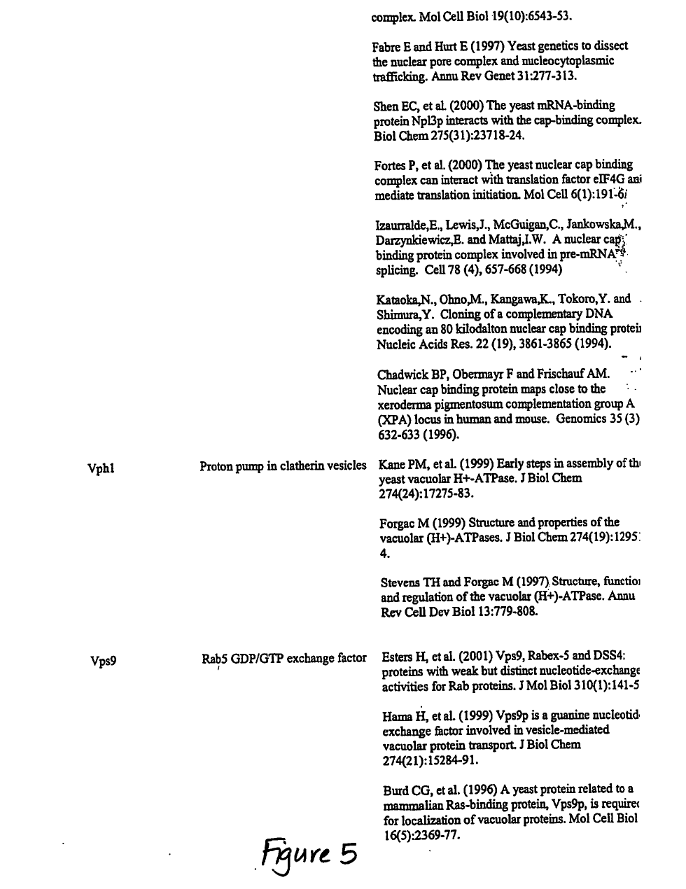

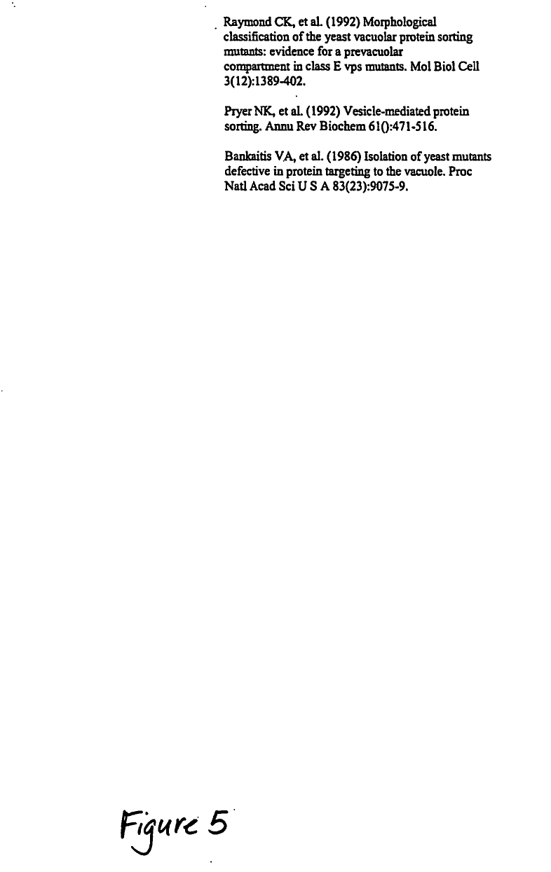

The results obtained in an exemplary screen on synthetic complete medium lacking histidine are shown in the photograph of FIG. 1B. Four knockout strains (listed to the right of the plate) were tested in triplicate (listed 1-3 above the plate) on each plate (after inducing retrotransposition). Two controls were included on each plate. The negative control was the wildtype 4743 stain (Winzeler et al., Science 285:901-906, 1999) carrying the pRS316 plasmid (Sikorski and Hieter, Genetics 122:19-27, 1989; lower left), and the positive control was the wildtype 4743 strain carrying the pAR100 Ty1 test plasmid (lower right). The positive control yielded a retrotransposition rate of approximately 1% under our test conditions, as judged by the appearance of His⁺ cells. The YMR032w strain (plated in the third row from the top) showed a clear decrease in Ty1 retrotransposition (in triplicate), and all three patches showed decreased numbers of His⁺ cells. An additional 24 plates were used to test each box of 96 deletion strains.

FIGS. 2A-2C represent transposition data for the chromatin mutants. The photographs in FIG. 2A show the results obtained when the ten chromatin mutants identified in our screen were tested. On each plate, the top row shows retrotransposition data from the original three transformants, the second row from the top shows retrotransposition in cells from the frozen stocks of those original three transformants, and the third row shows retrotransposition in cells of the three re-transformants. Negative and positive controls are shown at the bottom of each plate as described for FIG. 1B. Equivalent results were obtained with knockout strains that were independently generated using a LEU2 deletion cassette to delete the same genes in the 4741 strain background. The photograph of FIG. 2B illustrates a quantitative retrotransposition assay. Cells were scraped from the SC plus 5-Foa plate, diluted to an OD₆₀₀ of 1.0, and 2-fold serial dilutions were plated from left to right. FIG. 2C lists the fold changes for the chromatin mutants that were determined using the dilution assay depicted in FIG. 2B. Each mutant was tested in triplicate and the value shown represents the average of the three estimates. The fold-change estimates for all of the mutants in Table 1 were obtained. Fifty of the mutants yielded 3-8-fold changes and 51 yielded greater than 8-fold changes.

FIG. 3 is an illustration of the Ty1 retrotransposition cycle. The cycle begins with the transcription of Ty1 elements in the nucleus (step 1). Ty1 mRNAs are produced and exported to the cytoplasm (steps 2 and 3). The mRNAs are next translated to produce Ty1 Gag and Pol proteins (step 4). Ty1 virus-like particles are assembled and Ty1 mRNAs are packaged into these particles (step 5). The arrows exiting and entering the cell indicate the point at which retroviruses with envelope (ENV) genes can exit a cell and infect a new cell. The Ty1 mRNAs next are copied into double stranded (ds) cDNAs using reverse transcriptase (step 6). The cDNAs and Ty1 integrase (IN) then are imported back into the nucleus (step 7). The cDNAs finally are integrated into chromosomal DNA (step 8).

FIG. 4 is a compilation of human proteins homologous to the yeast host factors identified in the studies described below (the human host factors are represented by SEQ ID NOs:5-501). The GenBank™ accession number is provided for each sequence. The human proteins were identified by using the sequences of the yeast host factors as queries in a BLAST search of databases available through the National Center for Biotechnology Information (NCBI). Human homologs or homologs from other species can be identified using this resource. For example, one can identify homologs using the default parameters set by the search program (BLOSUM62 is the matrix; word length 3; gap penalty 11; gap extension penalty 1). Alternatively, one can accept matches under less stringent circumstances. Physical assays can also be performed to identify homologous sequences. For example, one can probe a cDNA library with a sequence that encodes one or more of the yeast or human host factors identified herein so that the sequence, which acts as a probe, hybridizes with potential target sequences in the library under conditions of high stringency. Highly homologous sequences will remain base-paired even following washing under conditions of high stringency (see the conditions of high stringency in Sambrook et al., Molecular Cloning—A Laboratory Manual, Cold Spring Harbor Laboratory Press, Cold Spring Harbor, N.Y., 1989).

FIG. 5 is a Table summarizing the functions of host factors. These functions are among those that can be assessed when determining whether a candidate compound inhibits the activity of a host factor.

### DETAILED DESCRIPTION

Ty1 is an LTR (long terminal repeat) retrotransposon in yeast that is a relative of vertebrate retroviruses (Boeke et al., The Molecular and Cellular Biology of Yeast Saccharomyces: Genome Dynamics, Protein Synthesis, and Energetics, J. R. Broach et al. Eds, pp 193-261, Cold Spring Harbor Laboratory Press, Cold Spring Harbor, N.Y, 1991). Like retroviruses, Ty1 encodes homologs of Gag and Pol proteins, forms virus-like particles, and transposes through an RNA intermediate using reverse transcriptase (Boeke et al., supra). Ty1 has a complex retrotransposition cycle that begins in the nucleus with the transcription of full-length Ty1 elements. As the cycle progresses, virus-like particles are assembled in the cytoplasm and, ultimately, double-stranded Ty1 cDNAs are generated from Ty1 mRNAs. The cycle is completed when these newly synthesized cDNAs integrate into chromosomal DNA in the nucleus of the host cell. Since the transposition cycle is complex and spans several intracellular compartments, it is likely to involve a wide range of host factors.

The human genome project has revealed that transposable genetic elements are abundant in the genomes of model organisms and humans. We have used bioinformatic, genomic, and biochemical tools to study the phenotypic effects of these transposons on the genomes of yeast and humans. Our work with the Ty1 retrotranposon of yeast has revealed that this transposon integrates very non-randomly in the yeast genome. Ty1 usually avoids integrating into the protein coding, gene-rich regions of the genome, and instead inserts preferentially upstream of tRNA genes and other genes that are transcribed by RNA polymerase III. Although this targeting system generally protects yeast genes from undesired transposon mutations, Ty1 does occasionally integrate into genes and cause mutations. To understand this targeting system better, we have conducted a functional genomics screen for factors that affect Ty1 transposition using the recently completed gene deletion collection generated by the Saccharomyces Deletion Project. We identified a number of cellular factors that influence Ty1. Our preliminary results indicate that transposon insertion polymorphisms (TIPS) and other types of Deletion/Insertion Polymorphisms (DIPs) represents a major source of genetic diversity in humans.

As noted, we identified host factors that influence Ty1 (and therefore function to facilitate Ty1 transposition) by screening the collection of mutants generated by the Saccharomyces Genome Deletion Project (Vmzeler et al., Science 285:901-906, 1999). An advantage of this approach is that all 46,200 yeast genes have been deleted in this single isogenic collection of knockout strains, allowing many genes to be tested in parallel for possible effects on a given process (in this case, Ty1 retrotransposition). Approximately 17% of the genes in yeast are “essential” and therefore produce lethal phenotypes upon gene deletion (Winzeler et al., Science 285:901-906, 1999). However, the remaining ˜83% of gene knockouts are viable and can, therefore, be tested readily for additional phenotypes.

Just over 100 genes (105) that influence many different aspects of the Ty1 retrotrasposition cycle were identified from uor analysis of 4,483 homozygous diploid deletion strains. Of these mutants, 46 had significantly altered levels of Ty1 cDNA. Thus, approximately half of the mutants apparently affected the early stages of retrotransposition leading up to the assembly of virus-like particles and cDNA replication, whereas the remaining half effected steps that occur after cDNA replication. Thus, if one specifically wished to identify an antiviral agent that acted by inhibiting either the early stages of the viral life or the later stages of the viral life cycle, the assays of the invention could be configured to assay the expression or activity of host factors affected at either of these relative times. Although most of the mutants retained the ability to target Ty1 integration to tRNA genes, two mutants had reduced levels of tRNA gene targeting. Thus, should one wish to search for antiviral agents that specifically interfered with gene targeting, the assay could be configured to assess the expression and/or activity of one of these two host factors.

As illustrated in FIG. 1A, we induced retrotransposition by growing cells carrying the test plasmid in a galactose-positive environment, and then assayed transposition by replicating to media lacking histidine. The test plasmid carries Ty1 and HIS3 sequences under the control of a Gal1 promoter. Because the deletion mutants lack an ability to grow in histidine, we were able to identify the genes that encode proteins required for retrotransposition by examining the ability of each of the mutant strains of yeast, carrying the test plasmid, to survive on histidine-free culture medium. If Ty1 integrates into the yeast genome, as evidenced by the cell's ability to survive on the histidine-free medium, we can conclude that the protein that is absent from the host deletion mutant is not one required for the retrotransposition. To the contrary, if the protein that is absent is required for retrotransposition, the yeast cells will not grow or will grow much less well. If there is no retrotransposition (because a protein required for that event has been effectively deleted from the mutant yeast cell), the cell will not express the exogenous HIS3 sequence and, consequently, will not be able to survive, or will have an impaired ability to survive, when plated on histidine-free medium. The assay also can detect deletions that cause increases in transposition by detecting increased numbers of HIS-positive cells on media lacking histidine.

The results we obtained represent a dramatic increase in the number of host factors that are known to affect Ty1 and provide information on the relationship between Ty1 and its yeast host. In addition, we discovered that many of the yeast host factors are homologous to human proteins, and we describe how factors from either or both sets can be used to identify antiviral agents (of course, homologs from other animals, such as rats, mice, or other rodents, rabbits, cats, dogs, sheep, cows, horses, goats, pigs, and non-human primates can be used in these methods as well).

The 105 genes that were identified in the initial study with Saccharomyces mutants are shown in Table 1.

TABLE 1
Deletion strains with moderate or strong changes in Ty1 retrotransposition
(retrotransposition levels measured in triplicate with dilution assays)

| | Gene Deleted (fold-change in retrotransposition (average of |
|---|---|
| Group (no. of genes) | three measurements)) |
| Chromatin (10) | ARD1 (−20.0); NAT1 (−32.0); SAP30 (−32.0); SIN1 (SPT2; |
| | −16.0); SIN3 (−16.0); SIN4 (−32.0); SPT4 (−32.0); SPT10 (−4.0); |
| | SPT21 (−16.0); STB5 (−32.0) |
| Chromatin Remodeling (4) | SNF2 (˜−10.0); SNF5 (˜−10.0); SNF6 (˜−10.0); SWI3 (˜−10.0) |
| DNA Repair (4) | APN1 (−9.3); MMS22 (−6.0); RAD52 (−4.0); XRS2 (−4.0) |
| Miscellaneous (27) | APG17 (−10.7); APL5 (−16.0); BEM1 (−8.0); BUD6 (−4.0); |
| | CHO2 (−4.0); CYK3 (−16.0); DCC1 (−12.0); ERV14 (−5.3); |
| | FYV3 (−16.0); HOF1 (CYK2; −16.0); JNM1 (−3.3); KCS1 |
| | (−6.7); KRE24 (−4.0); MAD2 (−3.3); MFT1 (−8.0); PAT1 |
| | (−16.0); NUM1 (−8.0); SCP160 (−4.0); SDF1 (−3.3); SEC22 |
| | (−9.3); SEC65 (+3.3); SMI1 (−8.0); SWA2 (−4.0); TPM1 (−8.0); |
| | TPS2 (−8.0); VPH1 (−8.0); VPS9 (−4.0) |
| Nuclear Transport (2) | NUP84 (−12.0); NUP133 (−5.3) |
| Protein Folding/ | CPR7 (−3.3); DBF2 (−8.0); DOA4 (−8.0); MCK1 (−32.0); NAT3 |
| Modification (8) | (−26.7); PFD1 (−4.6); SSE1 (−21.3); TCI1 (−3.3) |
| Ribosomes/Translation (9) | DBP3 (−8.0); RPL6A (−16.0); RPL14A (−8.0); RPL16B (−4.6); |
| | RPL19B (−13.3); RPL20B (−10.7); RPL21B (−6.7); RPP1A |
| | (−8.7); RPS10A (−10.7) |
| RNA Metabolism (8) | CBC2 (−24.0); DBR1 (−13.3); LEA1 (−16.0); LSM1 (−32.0); |
| | NOP12 (−13.3); RIT1 (−24.0); STO1 (CBC1; −32.0), YDL033c |
| | (−8.0) |
| Transcription (10) | CTK1 (−12.0); DEP1 (−37.3); HAC1 (−4.0); PHO23 (−6.0); |
| | POP2 (−13.3); RPA49 (−16.0); RTF1 (−9.3); SRB8 (−8.7); SSN2 |
| | (−8.0); SUB1 (−7.3) |
| Transcription/ | ELP2 (−6.0); ELP3 (−10.7); ELP4 (−6.0); ELP6 (−13.3); IKI3 |
| elongation (7) | (ELP1; −10.7); KTI12 (−4.0); THP2 (−6.0) |
| Unknown (16) | YBR077c (−6.0); YDL115c (−12.0); YDR496c (−10.7); |
| | YFL032w (−3.3); YGL250w (−5.3); YGR064w (−16.0); |
| | YKL053c-A (−4.0); YLR052w (−3.3); YLR322w (−8.7); |
| | YML010c-B (−16.0); YNL226w (−16.0); YNL228w (−16.0); |
| | YNL295w (−3.3); YOL159c (+4.0); YOR292c (−10.7); |
| | YPL080c (−4.7) |

At least 39 of the 105 factors have significant homology to human proteins (with BLASTp Expect values of <10⁻¹³; Table 2). This is not to say that human proteins that exhibit less homology with the yeast host factors are excluded from the invention or are less useful in the methods described herein. The yeast host factors, their human homologs, or homologous proteins similarly identified in other species (e.g., identified by searching sequence databases, using the identified yeast or human sequences as queries) can be used to screen compounds that affect (e.g., inhibit in any therapeutically useful way) human is retroviruses such as HIV (e.g., HIV-1 or HIV-2 of any subtype or lade). Such antiviral agents could, of course, prove effective in treating or preventing diseases associated with retroviruses (e.g., acquired immunodeficiency syndrome (AIDS).

TABLE 2
Ty1 host factors with significant matches to human host factors.

| | | |
|---|---|---|
| | Human | |
| Yeast | BLAST | |
| Protein | Score | Function/Phenotype |

Chromatin (4)

| | | |
|---|---|---|
| Ard1 | 2e−38 | N-terminal acetyltransferase |
| Nat1 | 1e−75 | N-terminal acetyltransferase |
| Sin3 | 5e−68 | Histone deacetylation |
| Spt4 | 2e−17 | Chromatin factor |

DNA Repair (1)

| | | |
|---|---|---|
| Rad52 | 3e−38 | Homologous recombination |

Miscellaneous (9)

| | | |
|---|---|---|
| Ap15 | 5e−92 | Vesicular trafficking |
| Erv14 | 4e−17 | Localized to ER-derived vesicles |
| Kcs1 | 9e−23 | Inositol hexakisphosphate kinase 3 |
| Mad2 | 8e−37 | Mitotic arrest deficient |
| Scp160 | 2e−33 | High density lipoprotein binding protein |
| Sdf1 | 3e−26 | Sporulation deficient |
| Sec22 | 1e−28 | Vesicular trafficking |
| Vph1 | 1e−169 | Proton pump in clatherin vesicles |
| Vps9 | 2e−20 | Rab5 GDP/GTP exchange factor |

Protein Folding/Modification (6)

| | | |
|---|---|---|
| Cpr7 | 3e−39 | Cyclophilin D |
| Dbf2 | 4e−56 | Serine/threonine kinase |
| Doa4 | 5e−47 | Ubiquitin specific protease 8 |
| Mck1 | 2e−69 | Protein kinase |
| Nat3 | 5e−28 | N-terminal acetyltransferase |
| Sse1 | 1e−120 | Hsp70 family |

Ribosomes/Translation (7)

| | | |
|---|---|---|
| Dbp3 | 2e−73 | RNA helicase |
| Rpl6a | 4e−28 | Ribosomal protein 6 |
| Rpl16b | 8e−51 | Ribosomal protein 13a |
| Rpl19b | 3e−34 | Ribosomal protein 19b |
| Rpl20b | 3e−42 | Ribosomal protein 18a |
| Rpl21b | 8e−40 | Ribosomal protein 21 |
| Rps10a | 1e−24 | Ribosomal protein S10 |

RNA Metabolism (5)

| | | |
|---|---|---|
| Cbc2 | 2e−35 | Nuclear cap binding protein subunit 2 |
| Dbr1 | 4e−66 | RNA lariat debranching enzyme |
| Lsm1 | 2e−17 | Lsm1 protein |
| Sto1/Cbc1 | 6e−13 | Nuclear cap binding protein subunit 1 |
| Ydl033c | 6e−41 | 5-methylaminomethyl-2-thiouridylate- |
| | | methyltranferase |

Transcription (2)

| | | |
|---|---|---|
| Ctk1 | 1e−69 | Ctk1 kinase |
| Pop2 | 2e−49 | CCR4 complex |

Transcription Elongation (4)

| | | |
|---|---|---|
| Elp2 | 3e−80 | Transcription elongation/Apoptosis inhibitor |
| Elp3 | 0 | Histone acetyltransferase |
| Iki1 (Elp1) | 4e−74 | RNA Polymerase II elongator subunit |
| Kti12 | 9e−15 | RNA Polymerase II elongator associated protein |

Unknown (1)

| | | |
|---|---|---|
| Ydr496c | 1e−38 | Unknown |

Human protein sequences homologous to the yeast host factors we identified initially are shown in FIG. 4. The sequences were identified by a conventional protein Blast™ search. These proteins and other host factors (as defined above) can be used to identify antiviral agents.

For example, antiviral agents can be identified by, first, identifying a compound that binds to or that inhibits the expression or activity of a host factor and, second, testing the compound for antiviral activity. For example, the method can be carried out by (a) exposing a host factor (or a number of host factors) to a candidate compound; (b) determining whether the candidate compound binds the host factors or inhibits the activity or expression of the host factors (a candidate compound that binds the host factors or inhibits the activity or expression of the host factors is a potential antiviral agent); (c) exposing a cell to the potential antiviral agent and a retrovirus; and (d) determining whether the potential antiviral agent inhibits the ability of the retrovirus to, for example, infect the cell, replicate therein, or exit the cell. A potential antiviral agent that inhibits the ability of the retrovirus to infect the cell or replicate therein (or that otherwise lessens the detrimental effect of a retroviral-associated disease on a patient) is an antiviral agent.

The candidate compound can be essentially any type of chemical or biological entity, and those of ordinary skill in the art will be able td identify sources of compounds to be tested in the methods described herein. There have been recent advances in high throughput screening, and those advances have given rise to a need for large numbers of compounds. Those of ordinary skill in the art routinely acquire and screen thousands of compounds in search of useful therapeutic agents. Compound libraries can be generated or obtained from a commercial supplier. For example, LeadQuest®, a library containing more than 80,000 compounds, can be obtained from Tripos (St. Louis, Mo.). Standard or custom made libraries can also be obtained from, for example, Ab Initio PharmaSciences (Basel, Switzerland), Affymax Research Institute (Santa Clara, Calif.), Array BioPharma, Inc. (Boulder, Colo.), Ascot Fine Chemical (Cambridge, England), ASDI Biosciences (Newark, DE), BioLeads GmbH (Heidelberg, Germany), and BIOMOL Research Laboratories, Inc. (Plymouth Meeting, Pa.). The compounds may be chiral compounds, small heterocycle motifs, peptidomimetics, or natural product derivatives.

When in the form of a library, the library can be a biological library (of, for example, peptides, oligonucleotides, or antibodies) or a spatially addressable parallel solid phase or solution phase library. Examples of methods for the synthesis of molecular libraries can be found in the art, for example in: DeWitt et al. (Proc. Natl. Acad. Sci. USA 90:6909, 1993); Erb et al. (Proc. Natl. Acad. Sci. USA 91:11422, 1994); Zuckermann et al. (J. Med. Chem. 37:2678, 1994); Cho et al. (Science 261:1303, 1993); Carrell et al. (Angew. Chem. Int. Ed. Engl. 33:2059, 1994); Carell et al. (Angew. Chem: Int. Ed. Engl. 33:2061, 1994); and Gallop et al. (J. Med. Chem. 37:1233, 1994).

Libraries of compounds may be presented in solution (e.g., Houghten, Bio/Techniques 13:412-421, 1992), or on beads (Lam, Nature 354:82-84, 1992), chips (Fodor, Nature 364:555-556, 1993), bacteria (U.S. Pat. No. 5,223,409), spores (U.S. Pat. Nos. 5,571,698; 5,403,484; and 5,223,409), plasmids (Cull et al. Proc. Natl. Acad. Sci. USA 89:1865-1869, 1992) or on phage (Scott and Smith, Science 249:386-390, 1990; Devlin, Science 249:404-406, 1990; Cwirla et al., Proc. Natl. Acad. Sci. USA 87:6378-6382, 1990; and Felici, J. Mol. Biol. 222:301-310, 1991).

Where inhibitors of gene expression are assayed, the inhibitor can be an antisense oligonucleotide or a sequence suitable for use in RNAi (e.g., a dsRNA, siRNA, or mRNA). RNAi (RNA interference) refers to the process of introducing a homologous double stranded RNA (dsRNA) into a cell to specifically target a gene sequence, resulting in null or hypomorphic phenotypes. RNAi is interesting because it is generally carried out with a is double stranded molecule, rather than single-stranded antisense RNA; it is highly specific; it is remarkably potent (only a few dsRNA molecules per cell may be required for effective interference); and the interfering activity (and presumably the dsRNA) can cause interference in cells and tissues far removed from the site of introduction.

Antisense oligonucleotides can also be tested as antiviral agents according to the methods of the invention and are well known in the art. Nucleic acids that hybridize to a sense strand (i.e., a nucleic acid sequence that encodes-protein, e.g. the coding strand of a double-stranded cDNA molecule) or to an mRNA, sequence are referred to as antisense oligonucleotides. While antisense oligonucleotides are “antisense” to the coding strand, they need not bind to a coding sequence; they can also bind to a noncoding region (e.g., the 5′ or 3′ untranslated region). For example, the antisense oligonucleotide can be complementary to the region surrounding the translation start site of an mRNA (e.g. between the −10 and +10 regions of a target gene of interest or in or around the polyadenylation signal). Moreover, gene expression can be inhibited by targeting nucleotide sequences complementary to regulatory regions (e.g., promoters and/or enhancers) to form triple helical structures that prevent transcription of the gene in target cells (see generally, Helene, Anticancer Drug Des. 6:569-84, 1991; Helene, Ann. N.Y. Acad. Sci. 660:27-36, 1992; and Maher, Bioassays 14:807-15, 1992). The sequences that can be targeted successfully in this manner can be increased by creating a so called “switchback” nucleic acid. Switchback molecules are synthesized in an alternating 5′-3′,3′-5′ manner, such that they base pair with first one strand of a duplex and then the other, eliminating the necessity for a sizeable stretch of either purines or pyrimidines on one strand of a duplex. Fragments having as few as 9-10 nucleotides (e.g., 12-14, 15-17, 18-20, 21-23, or 24-27 nucleotides) can be useful in the screening methods described herein.

Methods known in the art can also be used to determine whether a compound binds (e.g., specifically binds) a host factor or the gene that encodes it. Similarly, methods known in the art can be used to determine whether a compound inhibits one or more of the activities of the host factor. Some of the functions that can be examined, and the methods by which they may be assessed, are summarized in the Table shown as FIG. 5.

### EXAMPLES

Construction of the Test 1 Plasmid, pAR100

A Bam HI/NotI fragment carrying a Gal-Ty1-neo insert (Devine and Boeke, Genes Dev. 10:620-633, 1996) was cloned into the Bam HI and NotI sites of the pRS316 plasmid (Sikorski and Hieter Genetics 122:19-27, 1989) to generate the plasmid p3.1. APCR cassette carrying the HIS3 gene then was inserted into p3.1 at bases 6,168 to 7,080 of the Gal-Ty1-neo insert in both the forward and reverse orientations by homologous recombination in yeast (Kaiser et al. Methods in Yeast Genetics, Cold Spring Harbor Laboratory Press, Cold Spring Harbor, N.Y., 1994). The HIS3 cassettes were generated by PCR using the pRS403 plasmid (Sikorsid and Hieter Genetics 122:19-27, 1989) as a template and oligonucleotide primers with the following sequences:

(SEQ ID NO:1)

(SD516) 5′-TTACATTGCACAAGATAAAAATATATCATCATGAACAAT
AAAACTAGATTGTACTGAGAGTGCAC-3′,

(SEQ ID NO:2)

(SD517) 5′-CGCCGTCCCGTCAAGTCAGCGTAATGCTCTGCCAGTGTT
ACAACCCTGTGTCGGGTATTTCACACCG-3′,

(SEQ ID NO:3)

(SD518) 5′-TACATTGCACAAGATAAAAATATATCATCATGAACAATA
AAACTCTGTCGGGTATTTCACACCG-3′,
and

(SEQ ID NO:4)

(SD519) 5′-CGCCGTCCCGTCAAGTCAGCGTAATGCTCTGCCAGTGTT
ACAACCAGATTGTACTGAGAGTGCAC-3′.

The neo gene of Gal-Ty1-neo was replaced by the HIS3 gene using this strategy.

Transposition levels were similar for both constructs, and the reverse orientation construct, pAR100, was chosen for the screen (FIG. 1A).

The Ty1 Transposition Assay

The complete set of homozygous gene deletion strains (release 2) was obtained from Research Genetics (Huntsville, Ala.). A complete list of the genes tested can be viewed at the Research Genetics website. These deletion strains were transformed with the pAR100 test plasmid in batches of 96 following the order established by the Saccharomyces Genome Deletion Project using a lithium acetate method adapted to 96-well culture boxes (Winzeler et al., Science 285:901-906, 1999). All media were prepared as outlined previously (Kaiser et al. Methods in Yeast Genetics, Cold Spring Harbor Laboratory Press, Cold Spring Harbor, N.Y., 1994). Transformation reactions were plated on synthetic complete (SC) minus uracil (SC-U) medium and three independent transformants were patched onto SC-U medium. These plates were replica-plated to medium containing SC-U plus 2% galactose and incubated for four days at room temperature (24° C.) to induce transposition. They also were replica-plated to yeast peptone glycerol (YPG) medium to identify strains that could not support respiration (these strains were eliminated from further analysis). The SC-U plus galactose plates then were replica plated sequentially to: i) SC-U plus glucose, ii) yeast peptone dextrose (YPD), iii) SC plus glucose containing 1.2 g/L 5-Fluoroorotic acid (5-Foa), and iv) SC minus histidine (SC-H) plus glucose FIG. 1B). Plates were incubated overnight at 30° C. between each step.

Secondary Screens

All mutants that were positive in the initial screen were re-tested in a GAL1-lacZ reporter assay to identify host genes that influenced the GAL1 promoter used to induce transposition from the Ty1 test plasmid. Only a small fraction of the mutant candidates affected the GAL1 promoter as judged by the X-gal assay (Kaiser et al. Methods in Yeast Genetics, Cold Spring Harbor Laboratory Press, Cold Spring Harbor, N.Y., 1994), including deletions in several gal genes, and these were eliminated from further consideration. A second test was performed to determine whether the HIS3 Harker in the test Ty1 element was functioning in each putative Ty1 mutant. Host mutants that affected marker function would not be expected to yield a His⁺ phenotype after transposition and would be indistinguishable from actual Ty1 mutants. Thus, we tested whether each mutant candidate (carrying a Ty1 test plasmid) could support a His⁺ phenotype prior to the induction of transposition by replica-plating each strain to medium lacking histidine. A small number of strains were identified in this class, including strains carrying deletions in the known histidine biosynthesis genes (his1, his2, his4, his5, his6 and his7), and these were removed from further consideration.

Dilution Assays

Transposition levels were measured in triplicate for each mutant by plating serial dilutions of cells that had been induced for Ty1 transposition on medium that was selective for transposition events (SC-H) and on two control media (SC and SC-U). Cells were scraped from the SC plus 5-Foa patches into water and diluted to an OD₆₀₀ of 1.0. Two-fold dilutions were prepared in 96-well microtiter dishes and then plated on all three media using a multichannel pipettor. The SC plate served as a control for adjusting the cells to an OD₆₀₀ of 1.0, whereas the SC-U plate served as a control to ensure that the test plasmid had been eliminated successfully on the previous 5-Foa step. The number of cells growing at each dilution on the SC-H plate was compared to similar dilutions prepared from the wild-type strain and the fold-change was estimated (rounding to the nearest 2-fold dilution). The three independent measurements were averaged to produce the final fold-change value reported.

Targeting assays: The modified Ty1 element, placed under the control of the galactose-inducible GAL1 promoter, was used to test retrotransposition as described previously (Devine and Boeke, Genes. Dev. 10:620-633, 1996; Boeke et al., Cell 40:491-500, 1985). The yeast HIS3 gene was engineered into this test Ty1 element as a convenient marker for retrotransposition events in the his3Δ1 genetic background of the knockout collection (Winzeler et al., Science 285:901-906, 1999). Thus, if Ty1 transposed from the test plasmid into the yeast genome, it carried with it the HIS3 gene and conferred a His⁺ phenotype to an otherwise His⁻ strain (FIG. 1).

Using this plasmid-based assay, deletion strains with significantly altered levels of Ty1 retrotransposition were identified readily from the knockout collection (FIG. 1B). In fact, 2.3% of the yeast genes tested showed a Ty1 retrotrasposition phenotype, for a total of 105 mutants in the collection of 4,483. The vast majority of the mutants had decreased levels of retro-transposition (only yml105c and yol159c had increased levels). Transposition mutants were independently confirmed by re-transforming each strain with the Ty1 plasmid and re-testing it along with the original transformants and frozen stocks of the original transformants. The results of these comparisons were remarkably consistent (FIG. 2A).

All of the mutant candidates identified in our initial screen were subjected to two secondary tests designed to eliminate host genes that affected our assay rather than Ty1 retrotransposition itself. As expected, gal and his mutants were identified in these secondary screens, along with a few other mutants. Although gal and his mutants represented unwanted byproducts of our genomic screen, these mutants were fully expected to affect our assay and thus served as excellent internal controls for the accounting system of the knockout collection. The remaining 105 Ty1 host factor (thf) mutants were considered to have actual Ty1 retrotransposition phenotypes. These mutants clustered into ten groups according to the known functions of the genes (Table 1). The data for the chromatin mutants are shown in FIG. 2. Similar data were obtained for the remaining mutants in Table 1.

Although the patch assays alone indicated that the changes in retrotransposition levels generally were quite significant, quantitative retrotransposition assays also were performed on the mutants listed in Table 1. The results of these assays confirmed and extended the initial observations with the patch assays. Fifty of the mutants produced “moderate” (3- to 8-fold) changes in retrotransposition levels and fifty-one mutants produced “strong” (greater than 8-fold) changes in retrotransposition levels. An example of the assay is shown in FIGS. 2B and 2C. We also identified a number of mutants with “weak” (below 3-fold) changes in retrotrasposition levels, and these strains were omitted from the collection of mutants.

Ty1 cDNA analysis: Ty1 cDNA was measured by Southern hybridization analysis after a 48-hour induction in medium containing galactose. DNA was isolated from duplicate pAR100 transformants and analyzed as follows. After measuring the DNA concentration of each sample with a spectrophotometer, 10 μg of DNA was digested with the restriction endonuclease Afl II (which cuts 2,472 bp from the right end of Ty1-HIS3 cDNA) and run on a 1% agarose gel. The DNA was transferred to a nylon membrane (Osmonics) and then hybridized to a 1.4 kb probe that spanned the full HIS3 gene. Using this strategy, cDNA originating from the pAR100 donor plasmid was detected, but cDNA arising from genomic Ty1 copies was not detected. The HIS3 probe also hybridized to the linearized donor plasmid pAR100 and the his3Δ1 allele in the BY4743 strain background, thereby generating two additional bands in each lane (at 13 kb and 5 kb, respectively). These bands served as loading controls to ensure that equal amounts of DNA were analyzed in each lane. The prehybridization/hybridization buffer contained: 6× SSC, 0.01 M EDTA (pH 8.0), 5× Denhardt's solution, 0.5% SDS, and 100 μg/ml sheared, denatured salmon sperm DNA. The prehybridization, hybridization, and final wash steps were carried out at 65° C. The washed membranes were exposed to XAR5 film, and also were analyzed with a Fujix BAS1000 phosphoimager after exposing the membranes to phosphoimaging screens. Ty1 cDNA was measured in the duplicate samples by digital analysis of the scanned images, and the duplicates were averaged to obtain the final values reported. The Ty1 cDNA levels were considered to be altered from wild-type if the average of the duplicate measurements was below 50%, or greater than 200%, of wild type control cDNA levels.

Identification of Potential Homologs:

We next performed BLAST searches (Altschul et al., J. Mol. Biol. 215:403-410, 1990) to identify potential homologs of Ty1 host factors in other organisms. Full-length open reading frame translations were obtained for each of the genes listed in Table 1 from the Saccharomyces Genome Database and these sequences were used as BLAST queries against the non-redundant protein database at the National Center for Biotechnology Information (NCBI) using the default settings. Potential homologs were identified in a variety of organisms, including humans, with this approach, and the sequences of the human homologs are shown in FIG. 4 (SEQ ID NOs:5-501). Using a: BLAST Expect value cutoff of <10⁻¹³, thirty-nine of the 105 genes listed in Table 1 encoded proteins with significant matches to potential human homologs (Table 2). Similar results were obtained for mouse and other organisms.

As will be evident from the studies described above, 105 genes that presumably influence many different aspects of the Ty1 retrotransposition cycle were identified from our analysis of 4,483 homozygous deletion strains. These genes are known to participate in a wide range of cellular processes, and we classified then into 11 major groups based on the known functions of the encoded proteins.

Forty-six of the mutants identified in our screen had altered levels of Ty1 cDNA as measured by Southern hybridization analysis (Table 3). Forty-four of these mutants had decreased levels of cDNA, whereas two mutants had increased levels of cDNA. Since we eliminated mutants that affected the GAL1 promoter used in our Gal-Ty1 donor plasmid, none of the mutants is expected to affect the initial transcription step of the retrotransposition cycle in this system. However, several subsequent steps of the cycle must be completed before any Ty1 cDNA can be replicated, and mutants with diminished levels of cDNA could be deficient in any of these steps. Such steps include: i) the initial processing of Ty1 mRNA in the nucleus, ii) the export of Ty1 mRNA from the nucleus, ii) the translation of Ty1 proteins on ribosomes, and iv) the assembly of virus-like particles in the cytoplasm. The cDNA levels might also be affected by changes in the rate of cDNA replication or turnover.

Nine of the ten chromatin mutants examined in our study produced diminished levels of Ty1 cDNA compared to the BY4743 wild-type strain. One possible model to explain these results would be that these chromatin factors normally play an important role in protecting the Ty1 cDNA from degradation by nucleases. In the absence of these chromatin factors, the Ty1 cDNA is more vulnerable to nuclease digestion, and thus, Ty1 cDNA levels are decreased in such chromatin mutants. This model predicts the existence of an important chromatinized cDNA intermediate that is necessary for retro transposition. An alternative model would be that these chromatin factors regulate the expression of other genes that, in turn, affect cDNA replication or turnover. Such genes might include some of the other “early” genes identified in our study (Table 1). Additional studies will be required to differentiate between these (and perhaps other) models.

A number of other mutants in our collection also displayed decreased levels of cDNA and thus appear to affect early steps of the retrotransposition cycle. Within the RNA metabolism group, for example, both the cbc1 and cbc2 mutants had reduced levels of Ty1 cDNA. The Cbc1 and Cbc2 proteins form a “cap binding complex” that binds to the cap structure of cellular mRNAs (Fortes et al., Mol. Cell. Biol. 19:6543-6553, 1999). Therefore, Cbc1 and Cbc2 are likely to affect retrotransposition by binding to either Ty1 mRNA or to other cellular mRNAs that affect retrotransposition. Other mutants in the RNA metabolism group such as dbr1 also had decreased levels of Ty1 cDNA, consistent with previous reports (Karst et al., Biochem. Biophys. Res. Comm. 268:112-117; 2000). The lsm1 mutant in this group likewise had decreased levels of cDNA (Table 3). In contrast, the remaining four mutants within the RNA metabolism group had normal levels of cDNA.

We also identified 55 mutants that had normal levels of Ty1 cDNA (within a range of plus or minus two-fold of the wild type control levels) as judged by Southern analysis. These mutants are likely to affect one or more of the “late” steps of retrotransposition that occur after the production of cDNA. One of the first steps that must occur after cDNA replication is the nuclear localization of the newly-replicated Ty1 cDNA and integrase. Although it is presently unclear as to how the 6 kb Ty1 cDNA enters the nucleus, Ty1 integrase has a nuclear localization sequence that is required for retrotransposition (Kenna et al., Mol. Cell. Biol. 18:1115-1124, 1998; Moore et al., Mol. Cell. Biol. 18:1105-1114, 1998). Therefore, integrase enters the nucleus using the normal nuclear import machinery. Two known nuclear pore mutants, nup84 and nup133, were identified in our-screen that might affect this step of the retrotranposition cycle. In support of this model, the nup84 stain has normal levels of cDNA, indicating that it affects a late step of retrotranspostion. The nup133 mutant has increased levels of Ty1 cDNA that could, in principle, be caused by the accumulation of cDNA in the cytoplasm in the absence of efficient nuclear transport. Finally, the sin3 mutant identified in our study may also affect the nuclear localization of Ty1 components, since sin3 affects the nuclear import step of Tfl retrotransposition in Schizosaccharomyces pombe (Dang et al., Mol. Cell. Biol. 19:2351-2365, 1999).

TABLE 3
Mutants with altered cDNA levels

| | |
|---|---|
| Strain | cDNA level (% BY4743) |

Control

| | |
|---|---|
| BY4743 | 100.0 |

Chromatin

| | |
|---|---|
| ard 1 | 12.3 |
| nat1 | 22.9 |
| sap30 | 28.7 |
| sinI | 20.1 |
| sin4 | 22.2 |
| spt4 | 16.5 |
| spt10 | 15.9 |
| spt21 | 12.0 |
| stb5 | 14.6 |

DNA repair

| | |
|---|---|
| apn1 | 16.9 |

Nuclear transport

| | |
|---|---|
| Nup133 | 373.5 |

Miscellaneous

| | |
|---|---|
| bem1 | 19.6 |
| fyv3 | 15.5 |
| hof1 | 5.2 |
| jnm1 | 25.0 |
| kcs1 | 9.9 |
| mft1 | 15.6 |
| num1 | 15.1 |
| pat1 | 8.8 |
| scp160 | 36.3 |
| sec22 | 14.7 |
| tps2 | 18.3 |
| vps9 | 41.1 |

Protein Folding/Modification

| | |
|---|---|
| doa4 | 20.1 |
| mck1 | 7.1 |
| nat3 | 2.9 |

Ribosomes/Translation

| | |
|---|---|
| rp16a | 12.5 |
| rpl19b | 24.2 |
| rpl20b | 16.2 |
| rps10a | 6.1 |

RNA metabolism

| | |
|---|---|
| cbc1 | 12.1 |
| cbc2 | 18.4 |
| dbr1 | 18.1 |
| lsm1 | 13.6 |

Transcription

| | |
|---|---|
| ctk1 | 10.5 |
| pop2 | 12.9 |
| rtf1 | 9.4 |
| rpa49 | 8.1 |
| ssn2 | 21.7 |

Transcription elongation

| | |
|---|---|
| thp2 | 16.6 |

Unknown

| | |
|---|---|
| ydr496c | 9.7 |
| yor292c | 12.1 |
| ynl226w | 22.3 |
| ynl228w | 19.6 |
| yol159c | 351.1 |

After entering the nucleus, the cDNA is integrated into chromosomal DNA, primarily near tRNA genes. Despite the large number of host factors identified in our screen, only two factors were identified that affected tRNA gene targeting. A likely explanation for this seemingly small number of targeting mutants is that we only examined the non-essential yeast genes in our study. Because most of the RNA pol III transcription factors are encoded by essential genes, it is likely that we missed at least some targeting factors by focusing only on non-essential yeast genes. Additional screens, focused on essential genes, can be carried out to identify all of the host factors involved in targeting.

After cDNA integration, some level of DNA repair is likely to be required at the integration site, and perhaps at other sites in the yeast genome, to repair damaged DNA that is created during retrotransposition. Four DNA repair mutants were identified in our study. Three of the DNA repair mutants, mms22, rad52, and xrs2, had normal levels of cDNA, and therefore, affected late steps of the retrotransposition cycle. Such factors could be involved in repairing chromosomal DNA damage at integration sites or elsewhere in the genome. The remaining mutant, apn1, had significantly decreased levels of cDNA and thus affected an early step of the retrotransposition cycle. The Apn1 protein is an apurinic/apyrimidinic (AP) endonuclease that cleaves DNA at abasic sites in order to facilitate DNA repair. One possible model for Apn1 function would be that it is involved in cDNA repair prior to integration. If the cDNA were not repaired properly in an apn1 mutant, we believe the cDNA would be targeted for degradation.

Finally, most of the groups of genes listed in Table 1 contain both “early” and “late” mutants. Therefore, none of the groups appears to be devoted to a single step of the retrotransposition cycle. Nevertheless, some of the groups have a disproportionate number of mutants devoted to either early or late stages of the retrotranspostion cycle. For example, six of the seven transcription elongation mutants (elp1, elp2, elp3, elp4, elp6, and kti12) were found to affect the late stages of retrotransposition: All six of these “late” transcription elongation mutants could, in principle, affect retrotransposition by affecting the transcription of even a single “late” gene. Thus, our screen may have identified groups of genes that are involved in other processes (such as transcription elongation) that are necessary for retrotransposition. This might help to account for the large number of mutants identified in our study. Additional secondary screens and assays will be necessary to identify these groups and to determine how such factors work together to influence retrotransposition.

Although most of the mutants identified in our study retained the ability to target Ty1 integration to tRNA genes, two of the mutants identified, rit1 and ctk1, had diminished levels of tRNA gene targeting in our PCR assay. The Rit1 protein, which is an ADP-ribosylase, is known to modify the methionine tRNA that serves as a primer for Ty1 strong stop synthesis during cDNA replication (Chapman and Boeke, Cell 65:483-492, 1991; Astrom and Bystrom, Cell 79:535-546, 1994). Therefore, the rit1 mutant might have been expected to affect cDNA replication. Although the rit1 strain appeared to have slightly diminished levels of cDNA, the average for the duplicate cDNA measurements was considered to be within the “normal” range (70.5% of wild type). An alternative model would be that rit1 affects the efficiency of methionine tRNA cleavage from the end of the newly-replicated cDNA (Lauermann and Boeke, EMBO J. 16:6603-6612, 1997). If the cDNA lacked the appropriate end structure as a result of faulty end trimming in a rit1 mutant, it would not be expected to serve as a substrate for Ty1 integrase, and may not be integrated efficiently into the genome. Similar cDNA end mutants have been shown to form multimers that are integrated into the genome by homologous recombination rather than by the normal integrase-mediated mechanism (Sharon et al., Mol. Cell. Biol. 14:6540-6551, 1994). Thus, by interfering with cDNA end processing, rit1 might promote a shift towards integration by homologous recombination.

We also observed a decrease in tRNA gene targeting in the ctk1 mutant Ctk1p is a protein kinase that is known to regulate RNA polymerase II activity by phosphorylating the largest subunit of RNA polymerase II, Rpo21p (Patturajan et al., J. Biol. Chem. 274:27823-27828, 1999). One possible explanation for the diminished targeting in this mutant would be that ctk1 affects the RNA pol II transcription of a presently unknown host factor that is required for efficient targeting. Such factors might include proteins involved in RNA pol III transcription, for example. An alternative model would be that Ctk1p directly regulates RNA polymerase III activity. Since RNA pol III transcription, or an associated activity, is required for efficient tRNA gene targeting, altered phosphorylation of an RNA pol III subunit might be expected to have an impact on Ty1 integration.

A comparison of studies using Gal-Ty1 vs. chromosomal donor elements: Scholes et al. (Genetics 159:1449-1465, 2001) recently identified a large collection of Ty1 host mutants that had increased levels of Ty1 retrotransposition compared to wild type strains (Scholes et al., supra). We found little overlap between those Ty1 host mutants and the host factors identified in our screen. The most likely explanation for this result is that Scholes et al. screened for mutants with increased levels of retrotransposition using a chromosomal Ty1 donor element, whereas we screened for mutants with decreased levels of retrotransposition using a Gal-Ty1 donor plasmid. Decreases might be difficult to detect at the already low levels of retrotransposition attained with the chromosomal assay, whereas further increases may not be easily achieved at the relatively high levels of retrotransposition produced with a Gal-Ty1 donor plasmid assay. There also were several other technical differences between these two studies.

A number of additional host factors have been identified that affect the Ty1 retrotransposition cycle (Winston et al., Genetics 107:179-197, 1984; Chapman and Boeke, Cell 65:483-492, 1991; Boeke and Sandmeyer, In The Molecular and Cellular Biology of Yeast Saccharomyces: Genome Dynamics, Protein Synthesis, and Energetics, Eds. Broach et al., pp. 193-261, Cold Spring Harbor Laboratory Press, Cold Spring Harbor, N.Y., 1991; Rinkel and Garfikel, Genetics 142:761-776, 1996; Qian et al., Mol. Cell. Biol. 18:4783-4792, 1998; Huang et al., Genetics 151:1393-1407, 1999; Curcio and Garfinkel, Trends in Genetics 15:43-45, 1999; Bolton et al., Mol. Cell. Biol. 9:879-889, 2002). Upon comparing our genome-wide screen with these previous studies, we found that most of the factors identified in our screen were novel. Because our study was limited to the homozygous diploid deletion collection, we did not detect any host factors that were encoded by essential genes. We also did not generally detect spt mutants, because we used a GAL1 promoter instead of the normal LTR promoter to circumvent most of the spt mutants. Nevertheless, we did detect four spt mutants, spt2, spt4, spt10, and spt21, and all four of these had altered levels of Ty1 cDNA. Because these mutants did not affect the GAL1 promoter used on our Gal-Ty1 plasmid, these spt mutants must affect one of the remaining early steps of the retrotransposition cycle leading up to the assembly of virus-like particles and cDNA replication.

As expected, we identified the dbr1 gene in our screen and observed a decrease in retrotransposition that was similar to the decrease reported previously (Chapman and Boeke, Cell 65:483-492, 1991). We also identified the pmr1 gene in our screen (Bolton et al., Mol. Cell Biol. 9:879-889, 2002). Pmr1 is a calcium-transporting ATPase that has been shown to influence the production of Ty1 cDNA (Bolton et al., supra). However, pmr1 was set aside in our study because it did not grow well on YPG medium containing glycerol as the sole carbon source. We used YPG medium as a secondary screen to avoid mutants that could not support respiration and thus might not utilize galactose efficiently in our retrotransposition assay. A total of 86 strains were set aside for this reason, although only a small fraction also had retrotransposition phenoytypes. In the case of pmr1, it appears that this secondary screen was too stringent, and led to the elimination of a true positive (Bolton et al., supra). However, in most cases, problematic strains were set aside with this secondary screen, and such strains often grew poorly on at least one additional growth medium.

The steady-state levels of Ty1 cDNA are altered in many of the host factor mutants:

We next determined whether the host factor mutants in our collection produced normal levels of Ty1 cDNA. Because double stranded Ty1 cDNA is produced approximately midway through the retrotransposition cycle, it is a convenient measure of how far the retrotransposition cycle has progressed in a given mutant. Mutants with diminished levels of cDNA would be considered to affect the “early” steps of retrotransposition leading up to virus-like particle assembly and cDNA replication, whereas mutants with normal levels of cDNA would be considered to affect the “late” steps of retrotransposition that occur after cDNA production.

Interestingly, nine of the ten chromatin mutants examined were found to have significantly decreased levels of Ty1 cDNA compared to the wild type BY4743 control strain (FIG. 4A). Therefore, rather than affecting tRNA gene targeting, as we had originally postulated (Table 2), most of the chromatin mutants affected the production (or turnover) of Ty1 cDNA. Upon analyzing all of the mutants in our collection in duplicate by Southern analysis, we found a total of 44 strains with decreased levels of Ty1 cDNA (<50% of wild-type levels), and two mutants with increased levels of cDNA (>200% of wild-type levels; FIG. 4 and Table 3). The remaining 55 mutants bad normal levels of cDNA (between 50% and 200% of wild type levels; FIG. 4 and data not shown): Thus, almost half of the 101 mutants identified in our study affected the early steps of the Ty1 retrotransposition cycle leading up to the formation of virus-like particles and cDNA replication, whereas the remaining half affected the later steps that occur after cDNA replication.

A Prophetic Example

Both Ard1p and Nat1p were identified as yeast host factors that affect Ty1 in our functional genomics screen (described above). Ard1p and Nat1p have been found to work together as a heterodimer and are known to have protein acetyltransferase activity. One of the known substrate targets of the Ard1p/Nat1p heterodimer is a histone. Ard1p/Nat1p are also known to be required for telomeric silencing and silencing at the HML/HMR loci in yeast, and, in addition to the Ty1 phenotype mentioned above, also have several other known phenotypes. Human homologs of Ard1p and Nat1p have been identified (see the tables and figures herein).

Researchers can use existing chemical or drug libraries to screen for compound that bind to Ard1p and/or Nat1p, which may be produced in an expression system (e.g., E. coli) using a plasmid designed for that purpose. Tagged versions of these proteins could also be produced and used in conjunction with affinity chromatography columns that bind specifically to the tag for the purpose of purifying such proteins (GST or nickle columns, for example). Ard1p and/or Nat1p could also be expressed in a variety of other in vitro and in vivo systems such as: an in vitro transcription or translation system; an expression system in a vertebrate, such as the SV40 promoter on an Ebna/Orip vector, an expression system in insect cells, such as the Bacculovirus system; an expression system in yeast; etc. Ard1p/Nat1p also could be purified from cells as a native complex using biochemical techniques such as chromatography.

The purified proteins could be used to screen for compounds that bind to the protein. For example, the purified protein could be attached to a solid matrix in a multiple well format, and compound libraries could be screened for binding (one compound being tested per well). By using such high throughput methods, libraries of compounds could be screened. Alternatively, a protein could be exposed to a mixture of compound and those that were bound could be recovered and identified using methods known in the art, such as mass spectroscopy or NMR.

The proteins expressed as described above could also be used to generate antibodies that specifically recognize host factors. Should those antibodies be administered to human patients, they can be humanized.

The proteins expressed as described above could also be used to screen for comjpound that inhibit Ard1p and/or Nat1p acetyltransferase activity in vitro or in vivo. Alternatively, yeast strains containing intact Ard1p and Nat1p could be used to screen for compounds that inhibit Ard1p/Nat1p acetyltransferase activity. Such strains could also be used to screen for compounds that interfere with known phenotypes of Ard1p and/or Nat1p. Such screening could be done in conjunction with strains in which these genes have been deleted to confirm that Ard1p and/or Nat1p are the targets of such compounds.

An alternative approach is to introduce human homologs of Ard1p and/or Nat1p into yeast and screen for compounds in yeast that inhibit the human activities, including acetyltransferase activity and/or interference with telomeric silencing or other known phenotypes.

Murine homologs of these genes are also known and similar screens could be carried out with those homologs.

Once a compound has been identified, the compound can be tested for activity against a retrovirus. These tests can include applying the compound to human cells before or after the cells are infected with (or exposed to) a retrovirus. Viral titers could be measured using any method available in both treated and untreated controls.

Upon identifying a compound that inhibits viral infection or replication, analogs of such compounds (e.g., analogs bearing different R groups) could be made and tested for enhanced activity or decreased clinical side effects. Antibodies could be optimized for application to humans.

A number of embodiments of the invention have been described. Nevertheless, it will be understood that various modifications may be made without departing from the spirit and scope of the invention. Accordingly, other embodiments are within the scope of the following claims.

1. A method for identifying an antiviral compound, the method comprising:
(a) exposing a first cell that expresses a host factor to a candidate compound;
(b) determining whether the candidate compound inhibits the expression or activity of the host factor in the first cell, wherein a candidate compound that inhibits the expression or activity of the host factor in the first cell is a potential antiviral compound;
(c) exposing a second cell to the potential antiviral compound and a retrovirus; and
(d) determining whether the potential antiviral compound inhibits the ability of the retrovirus to infect or replicate within the second cell, wherein a potential antiviral compound that inhibits the ability of the retrovirus to infect the second cell is an antiviral compound.

2. The method of claim 1, wherein the first cell or the second cell is a cell in vivo.

3. The method of claim 1, wherein the first cell or the second cell is a cell in cell culture.

4. The method of claim 1, wherein the first cell is a yeast cell.

5. The method of claim 1, wherein the first cell is a bacterial cell.

6. The method of claim 5, wherein the bacterial cell is an E. coli cell.

7. The method of claim 1, wherein the first cell is a mammalian cell.

8. The method of claim 7, wherein the mammalian cell is a human cell.

9. The method of claim 1, wherein the first cell or the second cell is a cell of an established cell line.

10. The method of claim 8, wherein the second cell is a T lymphocyte.

11. The method of claim 1, wherein the first cell and the second cell are cells of the same type.

12. The method of any of claim 1, wherein the host factor is an N-terminal acetyltransferase, a histone deacetylase, a histone acetyltransferase, a chromatin factor, inositol hexakisphosphate kinase 3, a high density lipoprotein binding protein, a proton pump in clatherin-coated vesicles, a Rab5 GDP/GTP exchange factor, cyclophilin D, a serine/threonine kinase, ubiquitin specific protease 8, a heat shock protein, an RNA helicase, a ribosomal protein, a nuclear cap binding protein, an RNA lariat debranching enzyme, an Lsm1 protein, a nuclear cap binding protein subunit 1, a 5-methylaminomethyl-2-thiouridylate-methyltransferase, a Ctk1 kinase, a transcription elongation factor or an apoptosis inhibitor, an RNA polymerase II elongator subunit, or an RNA polymerase II associated protein.

13. The method of claim 1, wherein the host factor is a yeast host factor listed in Table 2, or a biologically active mutant or fragment thereof, a human host factor having an amino acid sequence represented by one of SEQ ID NOs.:1-501 or a biologically active mutant or fragment thereof.

14. The method of claim 13, wherein the host factor further comprises an affinity tag.

15. The method of claim 1, wherein the candidate compound is an antisense oligonucleotide or an siRNA.

16. The method of claim 1, wherein the candidate compound is an antibody.

17. The method of claim 1, wherein the candidate compound is a small molecule.

18. The method of claim 1, wherein the retrovirus is a human immunodeficiency virus (HIV).

19. The method of claim 18, wherein the HIV is HIV-1 or HIV-2.

20. The method of claim 1, wherein the retrovirus is a simian or feline immunodeficiency virus (SIV or FIV, respectively) or a human-simian chimeric virus (SHIV).

21. The method of claim 1, wherein the second cell is exposed to the potential antiviral agent before being exposed to the retrovirus.

22. The method of claim 1, wherein the second cell is exposed to the potential antiviral agent after being exposed to the retrovirus.

23. A method for identifying an antiviral compound, the method comprising:
(a) exposing a host factor to a candidate compound;
(b) determining whether the candidate compound binds to or inhibits the expression or activity of the host factor, wherein a candidate compound that binds to the host factor or inhibits the expression or activity of the host factor is a potential antiviral compound;
(c) exposing a cell to the potential antiviral compound and a retrovirus; and
(d) determining whether the potential antiviral compound inhibits the ability of the retrovirus to infect the cell, wherein a potential antiviral compound that inhibits the ability of the retrovirus to infect the cell is an antiviral compound.
